SEQUENCE LISTING (2 sequences)

SEQ ID NO: 1 - protein, 60 aa, Dendroaspis polylepis
RICYIHKASLPRATKTCVENTCYKMFIRTQREYISERGCGCPTAMWPYQTECCKGDRCNK

SEQ ID NO: 2 - protein, 60 aa, Dendroaspis angusticeps
WQPPWYCKEPVRIGSCKKQFSSFYFKWTAKKCLPFLFSGCGGNANRFQTIGECRKKCLGK

(19) United States
(12) Patent Application Publication
(10) Pub. No.: US 2018/0055813 A1
(43) Pub. Date: Mar. 1, 2018

(54) COMPOSITIONS AND METHODS FOR TREATING ATRIAL FIBRILLATION

(71) Applicant: Edge Therapeutics, Inc., Berkeley Heights, NJ (US)

(72) Inventors: Michael Kurz, Berkeley Heights, NJ (US); R. Loch Macdonald, Chatham Township, NJ (US)

(21) Appl. No.: 15/584,960

(22) Filed: May 2, 2017

Related U.S. Application Data
(60) Provisional application No. 62/338,316, filed on May 18, 2016.

(51) Int. Cl. A61K 31/343; A61K 31/28; A61K 45/06

(52) U.S. Cl. CPC A61K 31/343; A61K 45/06; A61K 31/28

(57) ABSTRACT

The described invention provides delivery systems, compositions and methods for reducing incidence or severity of atrial fibrillation in a subject at risk thereof, the method comprising providing a delivery system in a form that is malleable comprising a particulate formulation containing a plurality of particles comprising a therapeutic amount of an anti-arrhythmic agent; and a pharmaceutically acceptable carrier, wherein the therapeutic amount of the therapeutic agent is effective to reduce the incidence or severity of atrial fibrillation.

### CROSS-REFERENCE TO RELATED APPLICATIONS

This application claims the benefit of priority to U.S. Provisional Application No. 62/338,316, filed on May 18, 2016, the entire contents of which are incorporated by reference herein.

### FIELD OF INVENTION

The described invention relates to delivery systems, pharmaceutical formulations and therapeutic methods of use.

### BACKGROUND OF THE INVENTION

Anatomy of the Heart

The heart is a muscular organ weighing between 250-350 grams located obliquely in the mediastinum (membranous partition between the lungs). It functions as a pump, supplying blood to the body and accepting it in return for transmission to the pulmonary circuit for gas exchange (See, Marieb E N and Hoehn K, Human Anatomy and Physiology, Cardiovascular System, Benjamin Cummings. 8^{th} Ed. 2010; Noble A et al., The Cardiovascular System, Systems of Body Series, Churchill Livingstone. 2^{nd} Ed. 2010).

The heart contains four chambers that essentially make up two sides of two chamber (atrium and ventricle) circuits. The left side chambers supply the systemic circulation, and the right side chambers supply the pulmonary circulation. The chambers of each side are separated by an atrioventricular valve (A-V valve). The left-sided chambers are separated by the mitral (bicuspid) valve, and right-sided chambers are divided by the tricuspid valve. Blood flows through the heart in only one direction enforced by a valvular system that regulates opening and closure of valves based on pressure gradients (See, Marieb E N and Hoehn K, Human Anatomy and Physiology, Cardiovascular System, Benjamin Cummings. 8^{th} Ed. 2010; Noble A et al., The Cardiovascular System, Systems of Body Series, Churchill Livingstone. 2^{nd} Ed. 2010).

Cardiac muscle cells are branching, striated cells that contain myofibrils. Adjacent cardiac cells are connected by intercalated discs containing desmosomes (protein plaques in cell membranes linked by filaments) and gap junctions. The myocardium (muscular tissue of the heart) behaves as a functional syncytium (multinucleated mass resulting from multiple cell fusions of uninuclear cells) because of electrical coupling action provided by gap junctions. Cardiac muscle has abundant mitochondria that depend on aerobic respiration primarily to generate adenosine tri-phosphate (ATP) in order to provide energy for cellular function (See, Marieb E N and Hoehn K, Human Anatomy and Physiology, Cardiovascular System, Benjamin Cummings. 8^{th} Ed. 2010; Noble A et al., The Cardiovascular System, Systems of Body Series, Churchill Livingstone. 2^{nd} Ed. 2010).

### The Cardiac Cycle

The term “cardiac cycle” is used to refer to all or any of the mechanical events related to blood flow through or blood pressure developed from the beginning of one heartbeat to the beginning of the next. Blood pressure increases and decreases throughout the cardiac cycle. The frequency of the cardiac cycle is the heart rate. Every single ‘beat’ of the heart involves five major stages: (1)“late diastole,” which is when the semilunar valves close, the atrioventricular (Av) valves open and the whole heart is relaxed; (2) “atrial systole,” which is when the myocardium of the left and right atria are contracting, AV valves open and blood flows from atrium to ventricle; (3) “isovolumic ventricular contraction,” which starts when the ventricles begin to contract, the AV valves close, but the semilunar valves have yet to open so there is no change in volume; (4) “ventricular ejection,” which begins when pressure in the ventricles exceeds the pressure in the pulmonary artery (right ventricle) or aorta (left ventricle), forcing the semilunar valves open and allowing blood to flow from the ventricles to the lungs or systemic circulation; and (5) “isovolumic ventricular relaxation,” which begins when the ventricles stop contracting and begin to relax, the semilunar valves close because blood pressure in the arteries exceeds pressure in the relaxing ventricles, but pressure in the atria is too low to force open the AV valves to allow blood to enter the ventricles. The cardiac cycle is coordinated by a complex internal electrical conduction system which normally ensures that excitation, contraction and relaxation of the atria and ventricles occurs in a well-coordinated and efficient manner (i.e., as a “functional syncytium”). A group of specialized “pacemaker cells” cells, located in an area of the right atrium known as the “sino-atrial node,” spontaneously depolarize at a regular rate, sending an electrical impulse across the atria and initiating atrial contraction (Stage 2 of the Cardiac Cycle). Electrical signals also flow down the atrium via intermodal pathways to the AV node where the signal is briefly delayed to allow time for the atria to force as much blood as possible into the ventricles before ventricular contraction begins. At the end of stage 2, the electrical impulse exits the AV node via the Bundle of His, a very high-speed conduction pathway which transmits the impulse down the septum and branching to the left and right ventricles. This elaborate conduction system allows the entire ventricle to contract as a unit to generate the very high pressures needed to distribute blood throughout the entire body.

All cardiac myocytes are capable of spontaneously depolarizing under certain conditions, but only the specialized cells in the SA and AV nodes depolarize at regular rates. Because the pacemaker cells in the SA node fire at a faster rate, it normally determines the heart rate. If any of these signal generation or conduction pathways fails to function normally (due to injury or disease, for example), global cardiac function will be compromised. Disturbances in the normal electrical rhythm of the heart (arrhythmias) can cause life-threatening conditions ranging from the stroke-causing blood clots common with atrial fibrillation to ventricular fibrillation, which is fatal if not reversed within minutes.

Phases of the Cardiac Action Potential

The cardiac transmembrane potential consists of five phases: phase 0, upstroke or rapid depolarization; phase 1, early rapid repolarization; phase 2, plateau; phase 3, final rapid repolarization; and phase 4, resting membrane potential and diastolic depolarization. These phases are the result of passive ion fluxes moving down electrochemical gradients established by active ion pumps and exchange mechanisms. Each ion moves primarily through its own ion-specific channel. Impulses spread from one cell to the next without requiring neural input (See Braunwald's Heart Disease, 8th Edn, Libby P, Bonow R O, Mann F L and Zipes D P, Eds. Saunders (2008); pp. 737-744).

Phase 4: The Resting Membrane Potential.

The intracellular potential during electrical quiescence in diastole is −50 to −95 mV, depending on the cell type. Therefore, the inside of the cell is 50 to 95 mV negative relative to the outside of the cell because of the distribution of ions, such as potassium (K⁺), sodium (Na⁺) and chloride (Cl⁻). Because cardiomyocytes have an abundance of open K⁺ channels at rest, the cardiac transmembrane potential (in phase 4) is close to E_{K}. Deviation of the resting membrane potential from Eₖ is the result of movement of monovalent ions with an equilibrium potential greater than the Eₖ—for example, Cl⁻ efflux through activated chloride channels. Calcium (Ca²⁺) does not contribute directly to the resting membrane potential, but changes in intracellular free Ca²⁺ concentration can affect other membrane conductance values. For example, an increase in sarcoplasmic reticulum Ca²⁺ load can cause spontaneous intracellular Ca²⁺ waves, which in turn activate the Ca²⁺ dependent Cl⁻ conductance I_{Cl,Ca}, and thereby lead to spontaneous transient inward currents and concomitant membrane depolarization. Increases in Ca²⁺ can also stimulate the Na⁺/Ca²⁺ exchanger I_{Na/Ca}²⁺. This protein exchanges three Na⁺ ions for one Ca²⁺ ion; the direction is dependent on the Na⁺ and Ca²⁺ concentrations on the two sides of the membrane and the transmembrane potential difference. At resting membrane potential and during a spontaneous sarcoplasmic reticulum Ca²⁺ release event, this exchanger would generate a net Na⁺ influx, possibly causing transient membrane depolarization. Ca²⁺ has also been shown to active I_{K1} in cardiac myocytes, thereby indirectly contributing to cardiac resting membrane potential. Because of the Na—K pump, which pumps Na⁺ out of the cell against its electrochemical gradient and simultaneously pumps K⁺ into the cell against its chemical gradient, the intracellular K⁺ concentration remains high and the intracellular Na⁺ concentration remains low. The pump requires both Na⁺ and K⁺ to function and can transport three Na⁺ ions outward for two K⁺ ions inward. Therefore, the pump can be electrogenic and generate a net outward movement of positive charges. The rate of Na⁺—K⁺ pumping to maintain the same ionic gradients must increase as the heart rate increases, because the cell gains a slight amount of Na⁺ and loses a slight amount of K⁺ with each depolarization. Id.

Phase 0: Upstroke or Rapid Depolarization.

Action potentials in human cardiac fibers last several hundred milliseconds. When the stimulus is sufficiently intense to reduce membrane potential to a threshold value in the range of −70 to −65 mV for normal Purkinje fibers, an all or none response results. The upstroke of the cardiac action potential in atrial and ventricular muscle and His-Purkinje fibers is the result of a sudden increase in membrane conductance to Na⁺. The Na⁺ current is said to be regenerative; that is intracellular movement of a little Na⁺ depolarizes the membrane more, which increases conductance to Na⁺ more, which allows more Na⁺ to enter, etc. As this process is occurring however, [Na⁺] and positive intracellular charges increase and reduce the driving force for Na⁺. When the equilibrium potential for Na⁺ is reached, Na⁺ no longer enters the cell. In addition, Na⁺ conductance is time dependent, so that when the membrane spends some time at voltages less negative than the resting potential, Na⁺ conductance decreases. The process of channel inactivation is thought to be mediated by occlusion of the inner mouth of the pore by the peptide loop that connects domains III and IV. Id.

In normal atrial and ventricular muscle and in fibers in the His-Purkinje system, action potentials have very rapid upstrokes, with a large Vₘₐₓ, and are called fast responses. Action potentials in the normal sinus and AV nodes and many types of diseased tissue have very slow upstrokes with a reduced Vₘₐₓ and are called slow responses. Upstrokes of slow responses are mediated by a slow inward, predominantly L-type voltage gated Ca²⁺ current rather than the fast inward Na⁺ current. Recovery from inactivation also takes longer. The slow channel opens and closes more slowly than the fast channel, remains open for a longer time, and requires more time following a stimulus to be reactivated. Id.

Phase 1: Early Rapid Repolarization.

Following phase 0, the membrane repolarizes rapidly and transiently to almost 0 mV (early notch) partly because of the inactivation of I_{Na} and concomitant activation of several outward currents: the 4-aminopyridine-sensitive transient outward K⁺ current; the 4-aminopyridine-resistant Ca²⁺ activated chloride current; and Na⁺ outward movement through the Na⁺/Ca²⁺ exchanger operating in reverse mode. Id.

Phase 2: Plateau.

During the plateau phase, which may last several hundred milliseconds, membrane conductance to all ions falls. The plateau is maintained by the competition between outward current carried by K⁺ and Cl⁻ ions and inward current carried by Ca²⁺ moving through open L-type Ca²⁺ channels and Na+ being exchanged for internal Ca²⁺ by the Na⁺/Ca²⁺ exchanger operating in forward mode. After depolarization, potassium conductance falls to plateau levels as a result of inward rectification, (meaning the membrane conductance changes with voltage, such that K⁺ channels are open at negative potentials, but closed at less negative or positive voltages. Outward K⁺ movement carried by the slow component of the delayed rectifier K⁺ current also contributes to plateau duration. Id.

Phase 3: Final Rapid Repolarization.

Repolarization proceeds rapidly at least in part because of time dependent inactivation of I_{Ca,L}, with a decrease in the intracellular movement of positive charges, and activation of repolarizing K⁺ currents, including the slow and rapid components of the delayed rectifier K⁺ current I_{Ks} and I_{Kr} and the inwardly rectifying K⁺ currents I_{KI} and I_{K. Ach}, which all cause an increase in the movement of positive charges out of the cell. The net membrane current becomes more outward, and the membrane potential shifts to the resting potential. A small conductance Ca²⁺ activated K⁺-current, I_{K, Ca}, is expressed in human atrial myocytes, where it controls the time course of phase 3 repolarization.

Phase 4: Diastolic Depolarization.

Under normal conditions, the membrane potential of atrial and ventricular muscle cells remains steady throughout diastole. I_{KI} is the current responsible for maintaining the resting potential near the K⁺ equilibrium potential in atrial, His-Purkinje, and ventricular cells. I_{KI} is the inward rectifier and shuts off during depolarization. It is absent in sinus nodal and AV nodal cells. In other fibers found in certain parts of the atria, in the muscle of the mitral and tricuspid valves, in His-Purkinje fibers and in the sinus node and the distal portion of the AV node, the resting membrane potential does not remain constant in diastole but gradually depolarizes. If a propagating impulse does not depolarize the cell or group of cells, it can reach threshold by itself and produce a spontaneous action potential. The property possessed by spontaneously discharging cells is called phase 4 diastolic depolarization; when it leads to initiation of action potentials, automaticity results. The discharge rate of the sinus node normally exceeds the discharge rate of other potentially automatic pacemaker sites and thus maintains dominance of the cardiac rhythm.

The densities and properties of voltage-gated cardiac Na⁺, Ca²⁺, and K⁺ currents change during development, reshaping action potential waveforms and modifying the sensitivity to cardiac, as well as noncardiac, drugs. Alterations in the densities and properties of voltage-gated Na⁺, Ca²⁺, and K⁺ currents also occur in a number of myocardial disease states. These changes can lead directly or indirectly to arrhythmia generation, and can alter the sensitivity of individuals to the effects of cardiac and noncardiac drugs that influence the properties and/or the functional expression of these channels (Näbauer M, Käab S. Potassium channel down-regulation in heart failure. Cardiovasc Res 1998; 37: 324-334; Bolli R, Marbán E. Molecular and cellular mechanisms of myocardial stunning. Physiol Rev 1999; 79:609-634; Tomaselli G F, Marbán E. Electrophysiological remodeling in hypertrophy and heart failure. Cardiovasc Res 1999; 42:270-283; Van Wagoner D. Electrophysiological remodeling in human atrial fibrillation. Pacing and Clin Electrophysiol 2003; 26:1572-1575).

Voltage-Gated Ion Channels

Voltage-gated ion channels are a class of integral membrane proteins that allow the passage of selected inorganic ions across the cell membrane by opening and closing in response to changes in transmembrane voltage. (Sands, Z. et al., “Voltage-gated ion channels,” Current Biology, 15(2): R44-R47 (2005)). They have an important role in excitable neuronal and muscle tissues as they allow a rapid and coordinated depolarization in response to triggering voltage change.

Structure

Voltage-gated potassium, sodium and calcium ion channels are thought to have similar overall architectures. (Sands, Z. et al., “Voltage-gated ion channels,” Current Biology, 15(2): R44-R47 (2005)). They generally are composed of several subunits arranged such that there is a central pore through which ions can travel down their electrochemical gradients. The channels tend to be quite ion-specific, although similarly sized and charged ions may also travel through them to some extent.

Mechanism

Crystallographic structural studies of a potassium channel suggest that when a potential difference is introduced over the membrane, the associated electromagnetic field induces a conformational change in the potassium channel. The conformational change distorts the shape of the channel proteins sufficiently such that the channel, or cavity, opens to admit ion influx or efflux across the membrane, down its electrochemical gradient. This subsequently generates an electrical current sufficient to depolarize the cell membrane.

Voltage-gated sodium channels and calcium channels are made up of a single polypeptide with four homologous domains. Each domain contains 6 membrane spanning alpha helices. The voltage sensing helix, S4, has multiple positive charges such that a high positive charge outside the cell repels the helix and induces a conformational change such that ions may flow through the channel. Potassium channels function in a similar way, with the exception that they are composed of four separate polypeptide chains, each comprising one domain. The voltage-sensitive protein domain of these channels (the “voltage sensor”) generally contains a region composed of S3b and S4 helices, known as the “paddle” due to its shape, which appears to be a conserved sequence.

Voltage-Dependent Sodium Channels

Voltage-dependent sodium channels play an essential role in the initiation and propagation of action potentials in neurons and other electrically excitable cells such as myocytes and endocrine cells (See, e.g., Hille B: Ion Channels of Excitable Membranes. Sunderland, Mass.: Sinauer Associates Inc 2001). Although different sodium channels have broadly similar functional characteristics, small differences in properties distinguish different isoforms and contribute to their specialized functional roles in mammalian physiology and pharmacology (Yu F H and Catterall W A (2003) Genome Biology, 4:207, DOI: 10.1186/gb-2003-4-3-207).

Sodium Channel Subunits

Sodium channels consist of a highly processed a subunit, which is approximately 260 kDa, associated with auxiliary β subunits of 33-39 kDa (Catterall W A (2000), Neuron, 26(1): 13-25). For example, sodium channels in the adult mammalian heart contain a mixture of β₁-β₄ subunits, while sodium channels in adult skeletal muscle have only the β₁ subunit (Brackenbury W J and Isom L L (2011), Front Pharmacol, 2: 53; Isom L L. (2001), Neuroscientist, 7(1): 42-54). The pore-forming a subunit is sufficient for functional expression, but the kinetics and voltage-dependence of channel gating are modified by the β subunits. These auxiliary subunits are involved in channel localization and interaction with cell adhesion molecules, extracellular matrix and intracellular cytoskeleton. The a subunits are organized in four homologous domains (I-IV), each of which contain six transmembrane alpha helices (S1-S6) and an additional pore loop located between the S5 and S6 segments (Catterall W A (2000), Neuron, 26(1): 13-25). The pore loops line the outer entry to the pore while the S5 and S6 segments line the inner cavity and form an activation gate at the inner exit from the pore. The S4 segments in each domain contain positively charged amino acid residues (usually arginine) at every third position. These residues serve as gating charges and move across the membrane in order to initiate channel activation in response to depolarization. A short intracellular loop connecting homologous domains III and IV serves as the inactivation gate, folding into the channel structure and blocking the pore from the inside during sustained depolarization of the membrane.

Functionally, sodium channels are responsible for the generation and propagation of action potentials in excitable cells. They exist in three distinct states: (i) open; (ii) closed; and (iii) inactivated. In response to membrane depolarization, activation of sodium channels allows the rapid influx of Na⁺ ions leading to an upstroke of action potential. During depolarization, sodium channels rapidly (i.e., within a few milliseconds) become inactivated and Na⁺ influx declines. Upon repolarization to resting membrane potential, recovery of sodium channels from an inactivated state to a closed state occurs. Thus, the channels are “re-set” and available to open in response to membrane depolarization (See, Shah K U et al. (2010) CRIPS Vol. 11, No. 1, January-March, pp. 11-16; Cunnins T R et al. (2007) Pain, 131, pp. 243-257).

Voltage-dependent sodium channels are classified into two main groups based on sensitivity to tetrodotoxin (TTX): (i) TTX-sensitive; and (ii) TTX-resistant. TTX-sensitive channels include Nav1.1-Nav1.4, Nav1.6 and Nav1.7. TTX-resistant channels include Nav1.5, Nav1.8 and Nav1.9 (See, Shah K U et al. (2010) CRIPS Vol. 11, No. 1, January-March, pp. 11-16; Elliott A A and Elliott J R (1993) The Journal of Physiology, 463, pp. 39-56; Rush A M et al. (1998) The Journal of Physiology, 511, pp. 771-789).

Antagonists and Inhibitors of Sodium Channels

Phenytoin is a weak antagonist of voltage-dependent sodium channels at hyperpolarized membrane potentials and low rates of channel activation, but its inhibitory action is enhanced by sustained membrane depolarization. It is this voltage-dependent and frequency-dependent inhibition that allows phenytoin to suppress epileptic activity without substantially interfering with normal cognitive function (Rogawski M A (2004) Nat Rev Neurosci, 12: 564-72).

Carbamazepine is similar to phenytoin in that it inhibits voltage-dependent sodium channels in a voltage-dependent and frequency-dependent manner at clinically relevant concentrations. Compared to phenytoin, carbamazepine has a 3-fold lower affinity for depolarized channels but binds to them at a 5-fold faster rate (Rogawski M A (2004) Nat Rev Neurosci, 12: 564-72).

Lamotrigine is a voltage-dependent and frequency-dependent antagonist of voltage-dependent sodium channels. Additionally, this drug inhibits N-type and P-type high-voltage-activated Ca²⁺ channels and enhances K⁺ repolarizing currents (Rogawski M A (2004) Nat Rev Neurosci, 12: 564-72; Poolos N P et al. (2002) Nat Neurosci, 5; 767-74; Stefani A et al. (1996) Eur J Pharmacol, 307: 113-16; Zona C et al. (2002) Epilepsia, 43: 685-90).

Topiramate acts to depress sustained repetitive firing and voltage-gated Na⁺ currents. Additional mechanisms include an interaction with excitatory amino acid receptor-mediated transmission (Shank R P and Maryanoff B E (2008) CNS Neurosci Ther, 14: 120-42; Gryder D S and Ragawski M A (2003) J Neurosci, 23: 7069-74; Qian J and Noebels J L (2003) Epilepsy Res, 55; 224-33).

Riluzole, which is generally thought to be an antiglutamatergic drug, has also been characterized as a voltage-dependent sodium antagonist (Pittenger C et al. (2008) CNS Drugs, 22: 761-86).

Lacosamide inhibits voltage-dependent sodium channels at least partly by selectively enhancing channel slow inactivation (Curia G et al. (2009) CNS Drugs, 23: 555-68).

Lidocaine and mexiletine are effective drugs in treating neuropathic pain due to their local anti-inflammatory and anti-nociceptive effects (Challapalli V et al. (2005) Cochrane Database Syst Rev., CD003345). However, these effects cannot be explained solely by their action on voltage-dependent sodium channels (Hollmann M W and Durieux M E (2000) Anesthesiology, 93: 858-75; Mao J and Chen L L (2000) Pain, 87: 7-17). For example, systemic lidocaine enhances spinal inhibitory glycinergic neurotransmission independent of sodium channel inhibition (Muth-Selbach U et al. (2009) Eur J Pharmacol 613: 68-73).

Compounds that act to block voltage-gated sodium (Na⁺) channels of the heart are characterized as Class I antiarrhythmic compounds. All agents classified under this category restrict the fast sodium inward current responsible for the upstroke of the myocardial action potential. The main electrophysiological property of this group of antiarrhythmic compounds is reduction of the maximum rate of depolarization (MRD) in the myocardial cell (Muhiddin K A and Turner P, Postgraduate Medical Journal (1985) 61, 665-678). Other effects of this group include, without limitation, an increase in the threshold of excitability, a reduction of conduction velocity and prolongation of the effective refractory period (ERP). These changes are associated with inhibition of the spontaneous diastolic depolarization in automatic fibers, without a significant change in resting membrane potential (Singh et al. (1981a) International Journal of Clinical Pharmacology, Therapy and Toxicology, 19, 185; Keefe et al. (1981) Drugs, 22, 363). Class I antiarrhythmic compounds are subdivided into three subgroups, a, b and c: Class Ia drugs block fast inward sodium current (phase 0) during the depolarization of the cardiac cell membrane; drugs of this type prolong the action potential duration. Class Ia compounds include, but are not limited to, disopyramide, procainamide and quinidine Class Ib drugs are agents that decrease phase 0 of the action potential and shorten its duration. Non-limiting examples of Class Ib include ethmozin, lignocaine, mexiletine, phenytoin and tocainide. Class Ic drugs are compounds that slow phase 0, but have little or no effect on the duration of action potential (Keefe et al. (1981) Drugs, 22, 363; Hillis & Whiting (1983) British Medical Journal, 286, 1332). Class Ic compounds include, without limitation, ajmaline, aprindine, encainide, flecainide, lorainide and propafenone (Muhiddin K A and Turner P, Postgraduate Medical Journal (1985) 61, 665-678).

Voltage-Dependent Potassium Channels

Potassium channels are a diverse and ubiquitous family of membrane proteins present in both excitable and nonexcitable cells. Members of this channel family play critical roles in cellular signaling processes regulating neurotransmitter release, heart rate, insulin secretion, neuronal excitability, epithelial electrolyte transport, smooth muscle contraction, and cell volume regulation. Diseases of the heart, kidney, pancreas, and central nervous system that involve either mutation(s) in K⁺ channel gene(s) and/or altered regulation of K⁺ channel function have been identified (See, Shieh C-C et al. (2000) Parmacological Reviews, vol. 52, no. 4, 557-594).

The family of voltage-dependent potassium channels includes various delayed rectifier channels, including outward rectifier channels and inward rectifier channels. The term “rectifier channel” is derived from the fact that potassium conductance succeeds that of Na⁺ during action potential. The delayed rectifier current (I_{K}) is largely responsible for repolarization and consists of two components namely I_{Ks} (slowly activating current) and I_{Kr} (rapidly activating current). I_{Ks}, a slowly activating current, is enhanced by sympathetic hyperactivity. I_{Kr}, a rapidly activating current, is insensitive to sympathetic stimulation and is encoded by a Kv11.1 gene. Kv11.1, also known as human—ether a go go related gene (HERG), is found in ventricles and atria and functions to trigger repolarization of cardiac myocytes (See, Sandhiya S and Dkhar S A (2009) Indian J Med Res, 129, pp. 223-232).

Potassium Channel Subunits

Voltage-dependent potassium channels belong to a family of proteins characterized by the presence of a pore-forming subunit with a six transmembrane segment (S1-S6) topology in which the last two segments, linked by a pore loop, constitute the ion-permeation pore (Gutman et al. (2005) Pharmacol. Rev. 57, 473-508). The voltage-sensing domain is formed by the S1-S4 segments; S4 contains a high density of positively charged residues and is the main transmembrane voltage-sensing component (Yellen (1998) Q. Rev. Biophys. 31, 239-295; Yellen (2002) Nature 419, 35-42; Swartz (2004) Nat. Rev. Neurosci. 5, 905-916; Swartz (2008) Nature 456, 891-897; Ashcroft (2006) Nature 440, 440-447; Bezanilla (2008) Nat. Rev. Mol. Cell Biol. 9, 323-332). The K⁺ channel's four pore subunits form a tetrameric structure surrounding a central conduction pathway. At least three functional elements are found in K⁺ channels: (i) an anion conduction pore in which the ionic selectivity resides; (ii) a voltage sensor that detects changes in the electric transmembrane field, subsequently coupling its conformational states to the operation of the gate(s); and (iii) one or more gates that open and close in response to voltage (Barros et al. (March 2012) Vol. 3, Article 49, doi: 10.3389/fphar.2012.00049).

Antagonists and Inhibitors of Potassium Channels

Voltage gated potassium (Kv) channels in the myocardium are blocked by class III antiarrhythmic drugs, like amiodarone, sotalol, ibutilide, dofetilide, ambasilide, azimilide and bretylium. These drugs are effective in improving survival and reducing the incidence of sudden cardiac death due to ventricular fibrillation (Kudenchuk P J et al. (1999) N Engl J Med, 341: 871-8). Class III antiarrhythmic drugs act by delaying repolarization and prolonging action potential duration (APD). Anti-arrhythmics like dofetilide and ibutilide are both potent and highly selective for Kv channels. Dofetilide selectively blocks I_{Kr} and rapidly terminates atrial fibrillation (AF) and atrial flutter (AFl). However, ibutilide is non selective and blocks both Na⁺ and K⁺ channels (I_{Kr}). Other class III anti-arrhythmics like ambasilide and azimilide are non-selective antagonists as they block both I_{Kr} and I_{Ks} respectively (Lombardi F et al. (2006) Eur Heart J 27: 2224-31; Feng J et al. (1997) J Pharmacol Exp Ther, 281: 384-92). The drug sotalol is a non-selective competitive beta-adrenergic receptor antagonist that also exhibits Class III antiarrhythmic properties (Bertrix L et al. (1986) Cardiovascular Research, 20: 358-63; Edvardsson N et al. (1980) European Heart Journal, 1: 335-43).

Voltage-Dependent Calcium Channels

Voltage-dependent calcium channels (VDCC) are a group of voltage-gated ion channels that control calcium entry into cells in response to membrane potential changes. (Van Petegem F. et al., Biochemical Society Transactions, 34(5): 887-893 (2006)). At physiologic or resting membrane potential, VDCCs are normally closed, while they are activated (i.e., opened) at depolarized membrane potentials. Activation of particular VDCCs allows Ca²⁺ entry into the cell; muscular contraction, excitation of neurons, upregulation of gene expression, or release of hormones or neurotransmitters results, depending upon the cell type. (Catterall W. A. et al., “International Union of Pharmacology. XLVIII. Nomenclature and structure-function relationships of voltage-gated calcium channels,” Pharmacol. Rev., 57(4): 411-25 (2005); Yamakage M. et al, “Calcium channels—basic aspects of their structure, function and gene encoding; anesthetic action on the channels—a review,” Can. J. Anaesth., 49(2): 151-64 (2002)).

Voltage-dependent calcium channels are formed as a complex of several different subunits: α₁, α₂δ, β₁₋₄, and γ. The α subunit forms the ion conducting pore while the associated subunits have several functions including modulation of gating. (Dolphin A. C. “A short history of voltage-gated calcium channels,” Br. J. Pharmacol., 147 (Suppl 1): S56-62 (2006))

α1 Subunit

The α₁ subunit pore (about 190 kDa in molecular mass), the primary subunit necessary for channel functioning in the VDCC, consists of the characteristic four homologous I-IV domains containing six transmembrane α-helices each. The a subunit forms the Ca²⁺ selective pore, which contains voltage-sensing machinery and drug/toxin-binding sites. Ten a subunits that have been identified in humans. (Dolphin A. C. “A short history of voltage-gated calcium channels,” Br. J. Pharmacol., 147 (Suppl 1): S56-62 (2006)).

α2δ Subunit

The α₂δ gene encodes two subunits, α₂ and δ, which are linked to each other via a disulfide bond and have a combined molecular weight of 170 kDa. The α₂ is the extracellular glycosylated subunit that interacts the most with the al subunit. The δ subunit has a single transmembrane region with a short intracellular portion, which serves to anchor the protein in the plasma membrane. There are 4 α₂δ genes: CACNA2D1 (CACNA2D1), (CACNA2D2), (CACNA2D3), and (CACNA2D4). Co-expression of the α₂δ enhances the level of expression of the al subunit and causes an increase in current amplitude, faster activation and inactivation kinetics and a hyperpolarizing shift in the voltage dependence of inactivation. Some of these effects are observed in the absence of the beta subunit, whereas, in other cases, the co-expression of beta is required. The α₂δ-1 and α2δ-2 subunits are binding sites for at least two anticonvulsant drugs, gabapentin and pregabalin, that also find use in treating chronic neuropathic pain. (Dolphin A. C. “A short history of voltage-gated calcium channels,” Br. J. Pharmacol., 147 (Suppl 1): S56-62 (2006))

β Subunit

The β subunit (55 kDa) is an intracellular membrane-associated guanylate kinase (MAGUK)-like protein containing a guanylate kinase (GK) domain and an SH3 (src homology 3) domain. The guanylate kinase domain of the β subunit binds to the alpha subunit I-II cytoplasmic loop and regulates voltage-gated calcium channel (VGCC) activity. There are four known isoforms of the β subunit: CACNB1, CACNB2, CACNB3, and CACNB4. (Dolphin A. C. “A short history of voltage-gated calcium channels,” Br. J. Pharmacol., 147 (Suppl 1): S56-62 (2006))

Without being limited by theory, it has been postulated the cytosolic β subunit has a major role in stabilizing the final a subunit conformation and delivering it to the cell membrane by its ability to mask an endoplasmic reticulum retention signal in the a subunit. The endoplasmic retention brake is contained in the I-II loop of the α subunit that becomes masked when the β subunit binds. Therefore the β subunit functions initially to regulate the current density by controlling the amount of α subunit expressed at the cell membrane.

In addition to this potential trafficking role, the β subunit has the important functions of regulating activation and inactivation kinetics, and hyperpolarizing the voltage-dependence for activation of the α subunit pore, so that more current passes for smaller depolarizations. The β subunit acts as an important modulator of channel electrophysiological properties. The interaction between a highly conserved 18-amino acid region on the al subunit intracellular linker between domains I and II (the Alpha Interaction Domain, AIDBP) and a region on the GK domain of the β subunit (Alpha Interaction Domain Binding Pocket) is responsible for the regulatory effects exerted by the β subunit. Additionally, the SH3 domain of the β subunit also gives added regulatory effects on channel function, indicating that the β subunit may have multiple regulatory interactions with the al subunit pore. The α interaction domain sequence does not appear to contain an endoplasmic reticulum retention signal; this may be located in other regions of the I-II α1 subunit linker.

γ Subunit

The γ1 subunit is known to be associated with skeletal muscle VDCC complexes, but the evidence is inconclusive regarding other subtypes of calcium channel. The γ1 subunit glycoprotein (33 kDa) is composed of four transmembrane spanning helices; it does not affect trafficking, and, for the most part, is not required to regulate the channel complex. However, γ2, γ3, γ4 and γ8 also are associated with α-amino-3-hydroxy-S-methyl-4-isoxazolepropionic acid (AMPA) glutamate receptors, non-NMDA-type ionotropic transmembrane receptors for glutamate that mediate fast synaptic transmissions in the central nervous system (CNS) (an NMDA-type receptor is a receptor to which NMDA (N-methyl-D-aspartate) binds specifically). There are 8 genes for gamma subunits: γ1 (CACNG1), γ2 (CACNG2), γ3 (CACNG3), γ4 (CACNG4), (CACNG5), (CACNG6), (CACNG7), and (CACNG8). (Chu P. J. et al., “Calcium channel gamma subunits provide insights into the evolution of this gene family,” Gene, 280 (1-2): 37-48 (2002)).

Voltage dependent calcium channels, which vary greatly in structure and form, are classified as L-, N-, P/Q, T- and R-type according to their pharmacological and electrophysiological properties. These channel subtypes have distinct physiological functions. Molecular cloning has clarified the α1 subunit sequence of each channel; the α1 subunit has a specific role in eliciting activity in an individual channel. Nonetheless, selective antagonists for these channel subtypes are required for defining specific channels involved in each activity. For example, the neural N-type VDCCs are blocked by ω-conotoxin GVIA; the R-type VDCCs are resistant to other antagonists and toxins, are blocked by SNX-482, and may be involved in processes in the brain; and the closely related P/Q-type VDCCs are blocked by some Ω-agatoxins. The dihydropyridine-sensitive L-type VDCCs are responsible for excitation-contraction coupling of skeletal, smooth, and cardiac muscle, and for hormone secretion in endocrine cells, and are antagonized by phenylalkylamines and benzothiazepines.

Types of Voltage-Dependent Calcium Channels

L-Type Calcium Channels

L-type voltage-dependent calcium channels are opened when a smooth muscle cell is depolarized. This depolarization may be brought about by stretching of the cell, by an agonist-binding its G protein-coupled receptor (GPCR), or by autonomic nervous system stimulation. Opening of the L-type calcium channel causes influx of extracellular Ca²⁺, which then binds calmodulin. The activated calmodulin molecule activates myosin light-chain kinase (MLCK), which phosphorylates the myosin in thick filaments. Phosphorylated myosin is able to form cross bridges with actin thin filaments, and the smooth muscle fiber (i.e., cell) contracts via the sliding filament mechanism. (Yamakage M. et al, “Calcium channels—basic aspects of their structure, function and gene encoding; anesthetic action on the channels—a review,” Can. J. Anaesth., 49(2): 151-64 (2002))

L-type calcium channels also are enriched in the t-tubules of striated muscle cells, such as, skeletal and cardiac myofibers. As in smooth muscle, L-type calcium channels open when these cells are depolarized. In skeletal muscle, since the L-type calcium channel and the calcium-release channel (ryanodine receptor, or RYR) are mechanically gated to each other with the latter located in the sarcoplasmic reticulum (SR), the opening of the L-type calcium channel causes the opening of the RYR. In cardiac muscle, opening of the L-type calcium channel permits influx of calcium into the cell. The calcium binds to the calcium release channels (RYRs) in the SR, opening them (referred to as “calcium-induced calcium release” or “CICR”). Ca²⁺ is released from the SR and is able to bind to troponin C on the actin filaments regardless of how the RYRs are opened, either through mechanical-gating or CICR. The muscles then contract through the sliding filament mechanism, causing shortening of sarcomeres and muscle contraction.

R-Type Voltage Dependent Calcium Channels

R-type voltage dependent calcium channels (VDCC) are involved in regulating calcium flow. Without being limited by theory, R-type voltage-dependent Ca²⁺ channels that may be located within small diameter cerebral arteries may regulate global and local cerebral blood flow, since the concentration of intracellular free calcium ions determines the contractile state of vascular smooth muscle. (Yamakage M. et al, “Calcium channels—basic aspects of their structure, function and gene encoding; anesthetic action on the channels—a review,” Can. J. Anaesth., 49(2): 151-64 (2002)).

R-type voltage dependent calcium channel inhibitors are calcium entry blocking drugs whose main pharmacological effect is to prevent or slow the entry of calcium into cells via R-type voltage-gated calcium channels. The gene Caᵥ2.3 encodes the principal pore-forming unit of R-type voltage-dependent calcium channels being expressed in neurons.

N-Type Calcium Channels

N-type (‘N’ for “Neural-Type”) calcium channels are found primarily at presynaptic terminals and are involved in neurotransmitter release. Strong depolarization by an action potential causes these channels to open and allow influx of Ca²⁺, initiating vesicle fusion and release of stored neurotransmitter. N-type channels are blocked by ω-conotoxin. (Yamakage M. et al, “Calcium channels—basic aspects of their structure, function and gene encoding; anesthetic action on the channels—a review,” Can. J. Anaesth., 49(2): 151-64 (2002)).

P/Q-Type Calcium Channels

P-type (‘P’ for cerebellar Purkinje cells) calcium channels play a similar role to the N-type calcium channel in neurotransmitter release at the presynaptic terminal, and in neuronal integration in many neuronal types. They also are found in Purkinje fibers in the electrical conduction system of the heart (Winds, R., et al., J. Physiol. (Lond.) 305: 171-95 (1980); Llinds, R. et al., Proc. Natl. Acad. Sci. U.S.A. 86 (5): 1689-93 (1989)). Q-type calcium channel antagonists appear to be present in cerebellar granule cells. They have a high threshold of activation and relatively slow kinetics. (Yamakage M. et al, “Calcium channels—basic aspects of their structure, function and gene encoding; anesthetic action on the channels—a review,” Can. J. Anaesth., 49(2): 151-64 (2002)).

T-Type Calcium Channels

T-type (‘T’ for transient) calcium channel antagonists are low voltage-activated. They most often are found in neurons and cells that have pacemaker activity and in osteocytes. Mibefradil shows some selectivity for T-type over other types of VDCC. (Yamakage M. et al, “Calcium channels—basic aspects of their structure, function and gene encoding; anesthetic action on the channels—a review,” Can. J. Anaesth., 49(2): 151-64 (2002)).

Antagonists and Inhibitors of Voltage-Dependent Calcium Channels

Calcium channel antagonists are a class of drugs and natural substances having effects on many excitable cells of the body, such as the muscle of the heart, smooth muscles of the vessels or neuron cells. The primary action of many calcium channel antagonists is to decrease blood pressure, via L-type calcium channel blockade. (Survase, S. et al., “Actions of calcium channel antagonists on vascular proteoglycan synthesis: relationship to atherosclerosis,” Vasc. Health Risk Manag., 1(3): 199-208 (2005)).

Calcium channel antagonists act upon VDCCs in muscle cells of the heart and blood vessels. By blocking the calcium channel, they prevent increases of calcium concentrations in the cells when stimulated, which subsequently leads to less muscle contraction. In the heart, a decrease in calcium available for each beat results in a decrease in cardiac contractility. In blood vessels, a decrease in calcium results in less contraction of the vascular smooth muscle and therefore an increase in blood vessel diameter. The resultant vasodilation decreases total peripheral resistance, while a decrease in cardiac contractility decreases cardiac output. Since cardiac output and peripheral vascular resistance are the primary determinants of blood pressure, blood pressure drops.

Calcium channel antagonists do not decrease the responsiveness of the heart to input from the sympathetic nervous system. Since blood pressure regulation is carried out by the sympathetic nervous system (via the baroreceptor reflex), calcium channel antagonists allow blood pressure to be maintained more effectively than do β-blockers. However, because calcium channel antagonists result in a decrease in blood pressure, the baroreceptor reflex often initiates a reflexive increase in sympathetic activity leading to increased heart rate and contractility. The decrease in blood pressure also likely reflects a direct effect of antagonism of VDCCs in vascular smooth muscle, leading to vasodilation. A β-blocker may be combined with a calcium channel antagonist to minimize these effects.

Calcium channel antagonists may decrease the force of myocardial contraction, an effect that depends on the chemical class of antagonist. This is known as the “negative inotropic effect” of calcium channel antagonists. (Bryant, B. et al., “Pharmacology for health professionals,” 3rd Ed., Elsevier Australia (2010)). Most calcium channel antagonists are not the preferred choice of treatment in individuals with cardiomyopathy due to their negative inotropic effects. (Lehne, R., “Pharmacology for nursing care,” 7th Ed., St. Louis, Mo., Saunders Elsevier., p. 505 (2010)).

Some calcium channel antagonists exhibit a negative dromotropic effect in that they slow the conduction of electrical activity within the heart by blocking the calcium channel during the plateau phase of the action potential of the heart. This effect is known as a “negative dromotropic effect”. Some calcium channel antagonists can also cause a lowering of the heart rate and may cause heart block (which is known as the “negative chronotropic effect” of calcium channel antagonists). The negative chronotropic effects of calcium channel antagonists make them a commonly used class of agents for control of the heart rate in individuals with atrial fibrillation or flutter. (See for example, Murphy C. E. et al., “Calcium channel blockers and cardiac surgery,” J. Card. Surg., 2(2): 299-325 (1987)).

The antagonists for L, N, and P/Q-types of calcium channels are utilized in distinguishing channel subtypes. For the R-type calcium channel subtype, for example, w-agatoxin IIIA shows blocking activity, even though its selectivity is rather low. This peptide binds to all of the high voltage-activated channels including L, N, and P/Q subtypes (J. Biol. Chem., 275, 21309 (2000)). A putative R-type (or class αlE) selective antagonist, SNX-482, a toxin from the tarantula Hysterocrates gigas, is a 41 amino acid residue peptide with 3 disulfide linkages (1-4, 2-5 and 3-6 arrangement) (Biochemistry, 37, 15353 (1998), Peptides 1998, 748 (1999)). This peptide blocks the class E calcium channel (IC₅₀=15 nM to 30 nM) and R-type calcium current in the neurohypophysial nerve endings at 40 nM concentration. R-type (class E) calcium channel blocking activity is highly selective; no effect is observed on K⁺ and Na⁺ currents, and L, P/Q and T-type calcium currents. N-type calcium current is blocked only weakly 30-50% at 300 nM to 500 nM. Regionally, different sensitivity of R-type current to SNX-482 is observed; no significant effect on R-type current occurs in preparations of the neuronal cell body, retinal ganglion cells and hippocampal pyramidal cells. Using SNX-482, three α E-calcium subunits with distinct pharmacological properties are recognized in cerebellar R-type calcium channels (J. Neurosci., 20, 171 (2000)) Similarly, it has been shown that secretion of oxytocin, but not vasopressin, is regulated by R-type calcium current in neurohypophysial terminals (J. Neurosci., 19, 9235 (1999)).

Dihydropyridine calcium channel antagonists often are used to reduce systemic vascular resistance and arterial pressure, but are not used to treat angina (with the exception of amlodipine, which carries an indication to treat chronic stable angina as well as vasospastic angina) since the vasodilation and hypotension can lead to reflex tachycardia. This calcium channel antagonist class is easily identified by the suffix “-dipine.”

Phenylalkylamine calcium channel antagonists are relatively selective for myocardium. They reduce myocardial oxygen demand and reverse coronary vasospasm. They have minimal vasodilatory effects compared with dihydropyridines. Their action is intracellular.

Benzothiazepine calcium channel antagonists are an intermediate class between phenylalkylamine and dihydropyridines in their selectivity for vascular calcium channels. Benzothiazepines are able to reduce arterial pressure without producing the same degree of reflex cardiac stimulation caused by dihydropyridines due to their cardiac depressant and vasodilator actions.

L-type VDCC inhibitors are calcium entry blocking drugs whose main pharmacological effect is to prevent or slow entry of calcium into cells via L-type voltage-gated calcium channels. Examples of such L-type calcium channel inhibitors include, but are not limited to: dihydropyridine L-type antagonists such as nisoldipine, AHF (such as 4aR,9aS)-(+)-4a-Amino-1,2,3,4,4a,9a-hexahydro-4a14-fluorene, HCl), isradipine (such as 4-(4-Benzofurazanyl)-1,-4-dihydro-2,6-dimethyl-3,5-pyridinedicarboxylic acid methyl 1-methylethyl ester), calciseptine (such as isolated from (Dendroaspis polylepis ploylepis), H-Arg-Ile-Cys-Tyr-Ile-His-Lys-Ala-Ser-Leu-Pro-Arg-Ala-Thr-Lys-Thr-Cys-Val-Glu-Asn-Thr-Cys-Tyr-Lys-Met-Phe-Ile-Arg-Thr-Gln-Arg-Glu- Tyr-Ile-Ser-Glu-Arg-Gly-Cys-Gly-Cys-Pro-Thr-Ala-Met-Trp-Pro-Tyr-Gl-n-Thr-Glu-Cys-Cys-Lys-Gly-Asp-Arg-Cys-Asn-Lys-OH [SEQ ID NO: 1], Calcicludine (such as isolated from Dendroaspis angusticeps (eastern green mamba)), (H-Trp-Gln-Pro-Pro-Trp-Tyr-Cys-Lys-Glu-Pro-Val-Arg-Ile-Gly-Ser-Cys-Lys-Lys-Gln-Phe-Ser-Ser-Phe-Tyr-Phe-Lys-Trp-Thr-Ala-Lys-Lys-Cys-Leu-Pro- Phe-Leu-Phe-Ser-Gly-Cys-Gly-Gly-Asn-Ala-Asn-Arg-Phe-Gln-Thr-Ile-Gly-Glu-Cys-Arg-Lys-Lys-Cys-Leu-Gly-Lys-OH [SEQ ID NO: 2], Cilnidipine (such as also FRP-8653, a dihydropyridine-type inhibitor), Dilantizem (such as (2S,3S)-(+)-cis-3-Acetoxy-5-(2-dimethylaminoethyl)-2,3-dihydro-2-(4-methoxyphenyl)-1,5-benzothiazepine-4(5H)-one hydrochloride), diltiazem (such as benzothiazepine-4(5H)-one, 3-(acetyloxy)-5-[2-(dimethylamino)ethyl]-2,3-dihydro-2-(4-methoxyphenyl)-(+)-cis-monohydrochloride), Felodipine (such as 4-(2,3-Dichlorophenyl)-1,4-dihydro-2,6-dimethyl-3,5-pyridinecarboxylic acid ethyl methyl ester), FS-2 (such as an isolate from Dendroaspis polylepis polylepis venom), FTX-3.3 (such as an isolate from Agelenopsis aperta), Neomycin sulfate (such as C₂₃H₄₆N₆O₁₃.3H₂SO₄), Nicardipine (such as 1,4-Dihydro-2,6-dimethyl-4-(3-nitrophenylmethyl-2-[methyl(phenylmethylamino]-3,5-pyridinedicarboxylic acid ethyl ester hydrochloride, also YC-93, Nifedipine (such as 1,4-Dihydro-2,6-dimethyl-4-(2-nitrophenyl)-3,5-pyridinedicarboxylic acid dimethyl ester), Nimodipine (such as 4-Dihydro-2,6-dimethyl-4-(3-nitrophenyl)-3,5-pyridinedicarboxylic acid 2-methoxyethyl 1-methylethyl ester) or (Isopropyl 2-methoxyethyl 1,4-dihydro-2,6-dimethyl-4-(m-nitrophenyl)-3,5-pyridinedicarboxylate), Nitrendipine (such as 1,4-Dihydro-2,6-dimethyl-4-(3-nitrophenyl)-3,5-pyridinedicarboxylic acid ethyl methyl ester), S-Petasin (such as (3 S,4aR,5R,6R)-[2,3,4,4a,5,6,7,8-Octahydro-3-(2-propenyl)-4a,5-dimethyl-2-o-xo-6-naphthyl]Z-3′-methylthio-1′-propenoate), Phloretin (such as 2′,4′,6′-Trihydroxy-3-(4-hydroxyphenyl)propiophenone, also 3-(4-Hydroxyphenyl)-1-(2,4,6-trihydroxyphenyl)-1-propanone, also b-(4-Hydroxyphenyl)-2,4,6-trihydroxypropiophenone), Protopine (such as C₂₀K₉NO₅Cl), SKF-96365 (such as 1-[b-[3-(4-Methoxyphenyl)propoxy]-4-methoxyphenethyl]-1H-imidazole, HCl), Tetrandine (such as 6,6′,7,12-Tetramethoxy-2,2′-dimethylberbaman), (+/−)-Methoxyverapamil or (+)-Verapamil (such as 54N-(3,4-Dimethoxyphenylethyl)methylamino]-2-(3,4-dimethoxyphenyl)-2-iso-propylvaleronitrile hydrochloride), and (R)-(+)-Bay K8644 (such as R-(+)-1,4-Dihydro-2,6-dimethyl-5-nitro-442-(trifluoromethyl)phenyl]-3-pyridinecarboxylic acid methyl ester). The foregoing examples may be specific to L-type voltage-gated calcium channels or may inhibit a broader range of voltage-gated calcium channels, e.g. N, P/Q, R, and T-type.

Cardiac Arrhythmias

While the mechanisms responsible for cardiac arrhythmias are generally divided into categories of disorders of impulse formation, disorders of impulse condition, or combinations of both, currently available diagnostic tools do not permit unequivocal determination of the electrophysiological mechanisms responsible for many clinically occurring arrhythmias or their ionic bases (See Braunwald's Heart Disease, 8th Edn, Libby P, Bonow R O, Mann F L and Zipes D P, Eds. Saunders (2008); pp. 746-61).

Disorders of impulse formation are characterized by an inappropriate discharge rate of the normal pacemaker, the sinus node (e.g., sinus rates too fast or too slow for the physiological needs of the patient), or discharge of an ectopic pacemaker that controls atrial or ventricular rhythm. Pacemaker discharge from ectopic sites, often called latent or subsidiary pacemakers, can occur in fibers located in several parts of the atria, coronary sinus and pulmonary veins, AV valves, portions of the AV junction, and His-Purkinje system. Alternatively, the discharge rate of the latent pacemaker can speed inappropriately and usurp control of cardiac rhythm from the sinus node, which has been discharging at a normal rate, such as a premature ventricular complex or a burst of ventricular tachycardia. Disorders of impulse formation also can be caused by speeding or slowing of a normal pacemaker mechanism, e.g., phase 4 diastolic depolarization that is ionically normal or the sinus node or for an ectopic site such as a Purkinje fiber, but occurs inappropriately fast or slow) or by an ionically abnormal pacemaker mechanism. Id.

Automaticity is the property of a fiber to initiate an impulse spontaneously, without need for a prior stimulation, so that electrical quiescence does not occur. Abnormal automaticity can arise from cells that have reduced maximum diastolic potentials. Triggered activity is pacemaker activity that results consequent to a preceding impulse or series of impulses, without which electrical quiescence occurs. These depolarizations can occur before or after full repolarization of the fiber and are best termed early after depolarizations (EADS) when they arise from a reduced level of membrane potential during phases 2 (type 1) and 3 (type 2) of the cardiac action potential, or they are termed late or delayed after depolarizations (DADs) when they occur after completion of repolarization (phase 4) generally at a more negative membrane potential than that from which EADs arise. Not all after depolarizations may reach threshold potential, but, if they do, they can trigger another after depolarization and thus self-perpetuate. Id.

Disorders of Impulse Conduction

Conduction delay and block can result in bradyarrhythmias (occurring when the propagating impulse is blocked and is followed by asystole or a slow escape rhythm) or tachyarrhythmias (when the delay and block produce reentrant excitation). Factors involving both active and passive membrane properties that determine the conduction velocity of an impulse and whether conduction is successful include the stimulating efficacy of the propagating impulse, related to the amplitude and rate of rise of phase 0, the excitability of the tissue into which the impulse is conducted, and the geometry of the tissue. Id.

Electrical activity during each normal cardiac cycle begins in the sinus node and continues until the entire heart has been activated. Each cell becomes activated in turn, and the cardiac impulse dies out when all fibers have been discharged and are completely refractory. During this absolute refractory period, the cardiac impulse has “no place to go.” It must be extinguished and restarted by the next sinus impulse. If however, a group of fibers not activated during the initial wave of depolarization recover excitability in time to be discharged before the impulse dies out, they may serve as a link to re-excite areas that were just discharged and have now recovered from the initial depolarization. This process is referred to variously as reentry, reentrant excitation, circus movement, reciprocal or echo bear, or reciprocating tachycardia. Id.

Reentry has been implicated in atrial flutter, with the reentrant circuit confined to the right atrium, where it usually travels counterclockwise in a caudocranial direction in the interatrial septum and in a craniocaudal direction in the right atrial free wall. An area of slow conduction is present in the posterolateral to posteromedial inferior area of the right atrium, with a central area of block that can include an anatomical (inferior vena cava) and functional component. Id.

Reentry has also been implicated as a cause of atrial fibrillation, which is characterized (according to the multiple-wavelet hypothesis) by fragmentation of the wave front into multiple daughter wavelets. Electrical remodeling of the atria appears to be a key determinant for maintenance of atrial fibrillation Id.

### Atrial Fibrillation (AF)

Atrial fibrillation (AF) is the most common sustained cardiac arrhythmia, and its prevalence is increasing with aging of the population (Miyasaka Y, et al. (2006) Circulation, 114(2):119-125). It occurs when irritable foci, commonly located in the superior pulmonary veins, less commonly within the right atrium, and rarely in the superior vena cava or coronary sinus, cause rapid action potentials which result in an atrial heart rate between 400 and 600 beats per minute. AF is reflected in an ECG recording by the replacement of regular P-waves by an undulating baseline (reflecting continuous, rapid, spatially heterogeneous atrial activation) and irregular ventricular QRS complexes. Uncoordinated atrial activity prevents effective atrial contraction, leading to clot formation in the blind pouch atrial appendage. Irregular and inappropriately rapid ventricular activity interferes with cardiac contractile function. AF contributes significantly to population morbidity and mortality, and presently available therapeutic approaches have major limitations, including limited efficacy and potentially serious side effects such as malignant ventricular arrhythmia induction (Dobrev D and Nattel S. (2010) Lancet, 375(9721):1212-1223).

Pathophysiological Mechanisms and Relation to Atrial Fibrillation Forms

Focal Ectopic Activity

Normal atrial cells display typical voltage changes over time. They start at a negative intracellular membrane potential (the resting potential), become very positive when fired (depolarized) during phase 0, then go through a series of repolarizing steps to get back to the resting potential, at which they remain until the next action potential. Automatic activity occurs when an increase in time-dependent depolarizing inward currents carried by Na⁺ or Ca²⁺ (making the cell interior more positive) or a decrease in repolarizing outward currents carried by K⁺ (which keep the cell interior negative) causes progressive time-dependent cell depolarization. When threshold potential is reached, the cell fires, producing automatic activity. If automatic firing occurs before the next normal (sinus) beat, an ectopic atrial activation results. Delayed after depolarizations (DADs) result from abnormal diastolic leak of Ca²⁺ from the main cardiomyocyte Ca²⁺ storage organelle, the sarcoplasmic reticulum (SR). The principal Ca²⁺-handling mechanisms governing DAD-related firing (triggered activity). Ca²⁺ enters cardiomyocytes through voltage-dependent Ca²⁺ channels during the action potential plateau, triggering Ca²⁺ release from the SR via Ca²⁺ release channels known as ryanodine receptors (RyRs; RyR2 is the cardiac form). This systolic Ca²⁺ release is responsible for cardiac contraction. Following action potential repolarization, diastolic cardiac relaxation occurs when Ca²⁺ is removed from the cytosol back into the SR by a Ca²⁺ uptake pump, the SR Ca²⁺ ATPase (SERCA). DADs result from abnormal diastolic Ca²⁺ leak through RyR2 from the SR to the cytoplasm (Dobrev D, Voigt N, Wehrens X H. (2011) Cardiovasc Res, 89(4):734-743). Excess diastolic Ca²⁺ is handled by the cell membrane Na⁺,Ca²⁺-exchanger (NCX), which transports three Na⁺ ions into the cell per single Ca²⁺ ion extruded, creating a net depolarizing current (called transient inward current, or Iₜᵢ) that produces DADs. DADs that are large enough to reach threshold cause ectopic firing. Repetitive DADs cause focal atrial tachycardias (tachycardia is a heart rhythm >100 bpm). RyR2s are Ca²⁺ sensitive, and RyR2 leak results from SR Ca²⁺ overload or intrinsic RyR2 dysfunction. RyR2 function is modulated by channel phosphorylation: hyperphosphorylation makes RyRs leaky and arrhythmogenic (MacLennan DH, Chen S R. (2009) J Physiol., 587(pt 13):3113-3115). Loss or dysfunction of calsequestrin (CSQ), the main SR Ca²⁺-binding protein, exposes RyRs to excess free SR Ca²⁺ (MacLennan DH, Chen S R. (2009) J Physiol., 587(pt 13):3113-3115).

When action potential duration (APD) is excessively prolonged, cell membrane Ca²⁺ currents recover from inactivation and allow Ca²⁺ to move inward, causing early after-depolarizations (EADs). APD prolongation is spatially variable (Nattel S. (2002) Nature, 415(6868):219-226). Cells that generate EADs adjacent to more normally repolarizing cells raise the latter to threshold, causing them to fire and to initiate focal activity (Nattel S. (2002) Nature, 415(6868):219-226).

Reentry

Reentry requires a vulnerable “substrate.” Reentry substrates can be caused by altered electrical properties or by fixed structural changes. Reentry initiation usually requires a premature ectopic beat that acts as a trigger. For example, a premature beat arising at a branch point “A” results in an impulse conducting through the pathway leading to a recording point “B”, which is no longer refractory, but blocks in the pathway leading to a recording point “C” because of its longer refractory period. The premature impulse arrives at the distal end of previously refractory site “C” and attempts to reenter. Under normal conditions, without a reentry substrate, the conduction time from point “A” around the circuit through point “B” and back through point “C” is shorter than the refractory period, and the impulse cannot reenter. When APD is decreased, reducing the refractory period sufficiently, excitability is recaptured earlier and the reentering impulse can now sustain itself throughout the circuit. Slowed conduction can similarly allow the impulse to reenter (without APD abbreviation), because the more slowly conducting impulse leaves additional time for refractoriness to dissipate. A combination of atrial dilation and fibrosis creates longer potential conduction pathways for reentry, slows conduction, and imposes conduction barriers that favor the initiation and maintenance of multiple irregular reentry circuits that sustain AF (Nattel S, Burstein B, Dobrev D (2008) Circ Arrhythm Electrophysiol., 1(1):62-73.).

Relation of Basic Mechanisms to Pathophysiology

Ectopic activity can be transient, manifesting as isolated ectopic beats, or sustained, causing tachycardia. Any source of sustained rapid atrial tachycardia, whether an ectopic focus or regularly discharging reentry circuit, is called a driver. Drivers that discharge rapidly and regularly can cause irregular activity, characteristic of AF, if the emanating propagation waves break up in functionally heterogeneous atrial tissue, leading to fibrillatory conduction (Nattel S (2008) Circ Arrhythm Electrophysiol, 1(1):62-73; Berenfeld O et al. (2002) Circ Res., 90(11):1173-1180).

AF-related reentry occurs in two general forms: (a) single-circuit reentry, involving one primary reentry circuit driver; and (b) multiple-circuit reentry, involving multiple simultaneous dyssynchronous reentry circuits (Wakili R et al. (2011) J Clin Invest, 121(8): 2955-2968). The very rapid activation caused by AF produces atrial tachycardia remodeling (ATR) of atrial electrical properties, promoting functional reentry (Nattel S. (2002) Nature, 415(6868): 219-226). ATR effects vary in different atrial regions, causing spatial heterogeneity that promotes multiple-circuit reentry as well as structural remodeling (Nattel S. (2002) Nature, 415(6868): 219-226; Allessie M A, et al. (2001) Circulation, 2001; 103(5):769-77).

Clinical AF can be paroxysmal (i.e., self-terminating), persistent (i.e., requiring medical intervention to terminate), or permanent. Focal drivers, particularly ectopic sites in cardiomyocyte sleeves around the pulmonary veins, produce paroxysmal AF forms (9). Functional reentry substrates cause persistent AF that can be terminated, restoring normal rhythm. AF becomes permanent as the substrate becomes fixed and irreversible because of structural remodeling (Burstein B, Nattel S. (2008) J Am Coll Cardiol, 2008; 51(8):802-809).

Molecular Mechanisms Underlying Atrial Fibrillation

There are three principal categories of etiological contributors to AF: (i) heart disease; (ii) extra-cardiac factors; and (iii) genetic determinants which act through the pathophysiological mechanisms of focal ectopic activity, functional reentry substrates and fixed reentry substrates (Wakili R et al. (2011) J Clin Invest, 121(8): 2955-2968).

Heart Disease-Related Ectopic Activity

Atrial tissue samples from AF patients show abnormal SR Ca²⁺ handling, which causes spontaneous diastolic SR Ca²⁺ release events (Hove-Madsen L et al. (2004) Circulation, 2004; 110(11): 1358-1363; Liang X et al. (2008) Cardiology, 111(2): 102-110; Neef S et al. (2010) Circ Res, 106(6): 1134-1144). SR Ca²⁺ load is not increased, suggesting that spontaneous Ca²⁺ releases occur because of altered RyR2 function (Hove-Madsen L et al. (2004) Circulation, 2004; 110(11): 1358-1363; Neef S et al. (2010) Circ Res, 106(6): 1134-1144). RyR2 phosphorylation by PKA at Ser2808 and by Ca²⁺/calmodulin-dependent kinase II (CaMKII) at Ser2814 is increased in AF (Vest J A et al. (2005) Circulation, 111(16): 2025-2032; Neef S et al. (2010) Circ Res, 106(6): 1134-1144; Chelu M G et al., (2009) J Clin Invest, 119(7): 1940-1951; Greiser M et al., (2009) J Mol Cell Cardiol, 46(3): 385-394). CaMKII activity is normally autoinhibited. Ca²⁺-calmodulin binding removes autoinhibition, activates CaMKII, and causes autophosphorylation, which results in CaMKII Ca²⁺-independence Similar activation may result from CaMKII oxidation (Neef S et al. (2010) Circ Res, 106(6): 1134-1144). Changes in PKA and CaMKII RyR2 phosphorylation state may result not only from altered kinase activity, but also from alterations in phosphatases (Dobrev D et al. (2011) Cardiovasc Res, 89(4): 734-743). RyR2 phosphorylation increases its Ca²⁺ sensitivity, which in turn, enhances channel open probability (Chelu M G et al., (2009) J Clin Invest, 119(7): 1940-1951). Mice lacking RyR2-inhibitory FK506-binding protein 12.6 or with gain-of-function RyR2 mutations exhibit increased susceptibility to AF, along with increased atrial cell SR Ca²⁺ leak and triggered activity (Chelu M G et al., (2009) J Clin Invest, 119(7): 1940-1951; Sood S et al. (2008) Heart Rhythm, 5(7): 1047-1054). Angiotensin has been shown to promote AF via CaMKII oxidation and enhanced CaMKII phosphorylation of RyR2, in addition to its well-recognized effect of promoting structural remodeling (Burstein B, Nattel S (2008) J Am Coll Cardiol, 51(8): 802-809; Gassanov N et al. (2006) Cell Calcium, 39(2): 175-186). RyR2 dysfunction can also be induced by Ca²⁺ overload, resulting from phospholamban hyperphosphorylation, which removes phospholamban inhibition of SERCA and enhances SR Ca²⁺ uptake. Phospholamban hyperphosphorylation can result from enhanced PKA and/or CaMKII activity or from decreased phosphatase function. Decreased phosphatase function is primarily due to increased activity (i.e., hyperphosphorylation) of a phosphatase-inhibitory protein, I-1 (Dobrev D et al. (2011) Cardiovasc Res, 89(4): 734-743; El-Armouche A et al. (2006) Circulation, 114(7): 670-680).

Increased NCX expression and/or function is/are commonly observed in AF. This suggests that Iₜᵢ resulting from diastolic SR Ca²⁺ leak may be amplified and enhance the risk of DADs/triggered activity (Dobrev D et al. (2011) Cardiovasc Res, 89(4): 734-743; Neef S et al. (2010) Circ Res, 106(6): 1134-1144; El-Armouche A et al. (2006) Circulation, 114(7): 670-680; Lenaerts I et al. (2009) Circ Res, 105(9): 876-885). Cardiac inositol 1,4,5-trisphosphate (IP3) receptors IP3R2 act as Ca²⁺-transporting pathways that facilitate arrhythmogenic SR Ca²⁺ leak (Li X et al. (2005) Circ Res, 95(12): 1274-1281). IP3R2 expression is increased by ATR, suggesting that IP3R2-coupled amplification of atrial SR Ca²⁺ release events and related arrhythmogenesis may contribute to AF-related ectopic activity (Zhao Z H et al. (2007) Cardiology, 107(4): 269-276; Zima A V and Blatter L A (2004) J Physiol., 555(pt 3): 607-615).

AF is very common in patients with congestive heart failure (CHF). Focal drivers and triggered activity contribute to CHF-related AF (Ryu K et al. (2005) Electrophysiol., 16(12): 1348-1358; Stambler B S et al. (2003) Electrophysiol., 14(5): 499-507). CHF increases atrial SR Ca²⁺ load and reduces calsequestrin (CSQ) expression, thereby promoting spontaneous SR Ca²⁺ release (Yeh Y H et al. (2008) Circ Arrhythm Electorphysiol., 1(2): 93-102).

Coronary artery disease (CAD) increases AF risk approximately 3.5-fold (Krahn A D et al. (1995) Am J Med., 98(5): 476-484). Atrial ischemia promotes AF, in part by causing spontaneous SR Ca²⁺ release events and increasing NCX function, leading to triggered activity and atrial ectopy (Nishida K et al. (2011) Circulation, 123(2): 137-146; Sinno H et al. (2003) Circulation, 107(14): 1930-1936).

Extra-Cardiac Factors Contributing to Ectopic Activity

Adrenergic-dependent RyR2 phosphorylation promotes spontaneous SR Ca²⁺ leak (Ogrodnik J and Niggli E (2010) J Physiol., 588(pt 1): 225-242). Clinically relevant conditions that cause abnormal DAD-promoting Ca²⁺ handling may require adrenergic stimulation to elicit Ca²⁺ sparks and triggered arrhythmias (Nishida K et al. (2011) Circulation, 123(2): 137-146). Vagal activation may promote spontaneous arrhythmogenesis by reducing APD, allowing adrenergically induced after-depolarizations to induce ectopic activity in susceptible regions such as pulmonary veins (Chou C C et al. (2008) Heart Rhythm., 5(8): 1170-1177).

Genetic Contributors to Ectopic Activity

Genetic factors fall into two broad groups: (i) rare genetic variants with strong effects and a clear phenotype (single-gene mutations); and (ii) common genetic variants with weaker effects and a less overt phenotype (single nucleotide polymorphisms [SNPs]). For example, a mutation in the gene encoding the adapter protein ankyrin-B (long-QT syndrome-4 [LQT4]), which impairs targeting of multiple proteins to the cell membrane, alters Ca²⁺ handling, and leads to DADs/triggered activity (Mohler P J et al. (2003) Nature, 421(6923): 634-639). A predicted loss-of-function SNP in the gene encoding the SERCA-inhibitory protein sarcolipin is also associated with AF (Nyberg M T et al. (2007) Clin Chem Acta, 375(1-2): 87-91).

The first potential genetic cause of AF linked to an EAD mechanism was a loss-of-function SNP in the KCNE1 gene, which encodes the slow delayed-rectifier K⁺ channel (I_{Ks}) β-subunit minK involved in long QT syndrome-5 (LQTS) (Ehrlich J R et al. (2005) Cardiovasc Res., 67(3): 520-528). The classical EAD-promoting congenital long QT syndrome (LQTS) mutations also predispose to AF (Johnson J N et al. (2008) Heart Rhythm., 5(5): 704-709; Zellerhoff S et al. (2009) Electrophysiol., 20(4): 401-407). A single-gene mutation of the KCNA5 gene encoding the Kv1.5 α-subunit of the ultra-rapid delayed-rectifier I_{Kur} channel identified in an individual with idiopathic AF promotes EADs under adrenergic stimulation (Olson T M et al. (2006) Hum Mol Genet., 15(14): 2185-2191). Other gene variants in KNCA5 suggest a role for tyrosine kinases in AF (Yang T et al. (2010) Heart Rhythm., 7(9): 1246-1252). Recent work in a genetic LQTS mouse model directly implicated EAD mechanisms in AF (Blana A et al. (2010) Heart Rhythm., 7(12): 1862-1869; Lemoine M D et al. (2010) Circulation, 122(1): A11401).

Two gain-of-function Na⁺ channel (SCN5A) mutations associated with AF, but with no evidence for EAD-promoting APD prolongation, have been reported (Li Q et al. (2009) Biochem Biophys Res Commun., 380(1): 132-137; Makiyama T et al. (2008) J Am Coll Cardiol., 52(16): 1326-1334). These mutations may initiate AF by increasing Na⁺ channel availability and thereby promoting ectopic activity (Li Q et al. (2009) Biochem Biophys Res Commun., 380(1): 132-137).

Genetic loci on chromosomes 4q25, 16q22, and 1q21 have been associated with AF by a genome wide association study (GWAS). The first locus on chromosome 4q25 has since been replicated in two additional European cohorts (Sinner M et al. (2011) Cardiovasc Res., 89(4): 701-709; Gudbjartsson D F et al. (2007) Nature, 448(7151): 353-357). The closest gene is the pair-like homeodomain-2 gene (PITX2), which encodes a transcription factor that is crucial for cardiac development and pulmonary vein formation (Mommersteeg M T et al. (2007) Circ Res., 101(9): 902-909; Mommersteeg M T et al. (2007) Circ Res., 100(3): 354-362). Thus, this gene variant may be implicated in pulmonary vein ectopic sources of AF. An AF-associated SNP of the KCNN3 gene on chromosome 1q21 affects a Ca²⁺-activated K⁺ channel (Ellinor P T et al. (2010) Nat Genet., 42(3): 240-244). Research suggests that this mutation may act by abbreviating APD in pulmonary veins, promoting microreentry, or cause ectopic activity via EAD mechanisms (Ozgen N et al. (2007) Cardiovasc Res., 75(4): 758-769; Li N et al. (2009) J Physiol., 587(pt 5): 1087-1100).

Heart Disease-Related Functional Reentry

AF, as well as all very rapid atrial tachyarrhythmias, promote AF initiation and maintenance via atrial tachycardia remodeling (ATR) (Wijffels M C et al. (1995) Circulation, 92(7): 1954-1968; Shiroshita-Takeshita A et al. (2009) Cardiovasc Res., 81(1): 90-97). A major AF-promoting component of ATR is refractory period reduction due to APD abbreviation. Decreased depolarizing L-type Ca²⁺ current (I_{Ca,L}), along with increased repolarizing inward-rectifier K⁺ currents, background AA and constitutively active acetylcholine-dependent current (kAchc), underlie ATR-induced APD shortening (Yue L et al. (1997) Circ Res., 81(4): 512-525; Van Wagoner D R et al. (1999) Circ Res., 85(5): 428-436; Christ T et al. (2004) Circulation, 110(17): 2651-2657; Ehrlich J R et al. (2004) J Physiol., 557 (pt 2): 583-597; Dobrev D et al. (2001) Circulation, 104(21): 2551-2557; Dobrev D et al. (2005) Circulation, 112(24): 3697-3706; Cha T J et al. (2006) Circulation, 113(14): 1730-1737; Voigt N et al. (2007) Cardiovasc Res., 74(3): 426-437).

The molecular basis of ATR-induced I_{Ca,L} reduction is complex (Wakili R et al. (2011) J Clin Invest., 121(8): 2955-2968). Rapid atrial activation causes Ca²⁺ loading, which activates the Ca²⁺/calmodulin/calcineurin/nuclear factor of activated T cells (NFAT) system, causing transcriptional downregulation of the Cav1.2 α-subunit (Yue L et al. (1999) Circ Res., 84(7): 776-784; Qi X Y et al. (2008) Circ Res., 103(8): 845-854). Other potential contributors include downregulation of accessory β₁-, β₂ₐ-, β_{2b}-, β₃-, and α₂δ₂-subunits, Ca²⁺ channel dephosphorylation due to type 1 (PP1) and type 2A (PP2A) serine/threonine protein phosphatase activation, and enhanced Cav1.2 α-subunit S-nitrosylation (Gaborit N et al. (2005) Circulation, 112(4): 471-481; El-Armouche A et al. (2006) Circulation, 114(7): 670-680; Christ T et al. (2004) Circulation, 110(17): 2651-2657; Bosch R F et al. (2003) J Am Coll Cardiol., 41(5): 858-869; Grammer J B et al. (2001) Basic Res Cardiol., 96(1): 82-90; Carnes C A et al. (2007) J Biol Chem., 282(38): 28063-28073). MicroRNAs (miRNAs) appear to play a major role in AF with increased miR-328 implicated in ATR-induced I_{Ca,L} downregulation (Wang Z et al. (2011) Cardiovasc Res., 89(4): 710-721; LU Y et al. (2010) Circulation, 122(23): 2378-2387). Impaired Cav1.2 protein trafficking induced by a zinc-binding protein (ZnT-1) may contribute to I_{Ca,L} reduction (Levy S et al. (2009) J Biol Chem., 284(47): 32434-32443).

Inward-rectifier K⁺ currents play an important role in AF maintenance, by both reducing APD and accelerating arrhythmia-maintaining rotors through hyperpolarization of atrial cells, thereby removing voltage-dependent I_{Na} inactivation (Pandit S V et al. (2005) Biophys J., 88(6): 3806-3821). I_{K1} increases due to increased expression of the underlying Kir2.1 subunit, likely due to reduced levels of Kir2.1-inhibitory miRNAs, particularly miR-1, miR-26, and miR-101 (Cha T J et al. (2005) Circulation, 111(6): 728-735; Voigt N et al. (2008) Cardiovasc Res., 77(1): 35-43; Girmatsion Z et al. (2009) Heart Rhythm., 6(12): 1802-1809). Stronger I_{K1} dephosphorylation (i.e., activation) due to increased PP1 and PP2A activity might also contribute (Luo X et al. (2010) Circulation, 122: A19435).

Muscarinic cholinergic receptor-mediated I_{K,ACh} activation is reduced in AF (Karle C A et al. (2002) Circulation, 106(12): 1493-1499). Loss of receptor-mediated I_{K,ACh} channel control is associated with increased agonist-independent (“constitutive”) I_{K,AChc} (Karle C A et al. (2002) Circulation, 106(12): 1493-1499). Increased I_{K,AChc} is due to enhanced single I_{K,ACh} channel opening frequency, without changes in single-channel conductance, density, or voltage dependence (Karle C A et al. (2002) Circulation, 106(12): 1493-1499). Enhanced I_{K,ACh} channel opening is related to altered protein kinase C (PKC) isoform balance, with increased I_{K,ACh}-stimulatory novel isoform function and reduced inhibitory classical isoform influence (Makary S et al. (2009) Circulation, 120: S664). Blockade of I_{K,AChc} suppresses ATR-induced APD abbreviation and AF promotion (Cha T J et al. (2006) Circulation, 113(14): 1730-1737).

Conduction slowing promotes reentry. Gap junctions are crucial for cell-to-cell coupling and conduction; however, information regarding gap-junctional remodeling in AF is inconsistent (Gaborit N et al. (2005) Circulation, 112(4): 471-481; Wakili R et al. (2011) J Clin Invest., 121(8): 2955-2968). Connexin alterations likely vary with AF duration, underlying pathology, and species (Nishida K et al. (2010) Europace, 12(2): 160-172). Clinical evidence suggests that connexin-40 gene variants predispose to AF (Ausma J et al. (2003) Circulation, 107(15): 2051-2058; Firouzi M et al. (2004) Circ Res., 95(4): e29-e33; Gollob M H et al. (2006) N Engl J Med, 354(25): 2677-2688). The importance of atrial connexin-43 dephosphorylation and lateralization in CHF is unclear, since CHF-induced conduction slowing and AF promotion remain following CHF recovery, despite full resolution of connexin abnormalities (Burstein B et al. (2009) Circ Res., 105(12): 1213-1222).

Atrial ischemia/infarction produces a substrate for AF via functional reentry circuits stabilized by a line of conduction block in the ischemic zone (Nishida K et al. (2011) 123(2): 137-146; Sinno H et al. (2003) Circulation, 107(14): 1930-1936). In acute ischemia, the conduction block is likely related to gap junction uncoupling (Sinno H et al. (2003) Circulation, 107(14): 1930-1936).

Extra-Cardiac Factors Contributing to Functional Reentry

Clinical AF is more likely to occur under vagotonic conditions, with AF in some patients clearly vagally dependent (Sossalla S et al. (2010) 55(21): 2330-2342). Vagal nerve endings release acetylcholine, stimulating cardiac muscarinic cholinergic receptors that activate I_{K,ACh}. The effects of vagal activation are spatially heterogeneous, promoting the initiation and stabilization of multiple AF-maintaining reentrant rotors (Sossalla S et al. (2010) 55(21): 2330-2342).

Genetic Factors Contributing to Functional Reentry

The most common AF-promoting genetic paradigm is APD shortening caused by gain-of-function K⁺ channel mutations. The first gene mutation linked to AF caused gain of function in the α-subunit (KCNQ1) of the slow delayed rectifier I_{Ks} (Chen Y H et al. (2003) Science, 299(5604): 251-254). Other genes with AF-inducing gain-of-function K⁺ channel mutations include KCNH2, KCNJ2, and KCNE2, corresponding to ion channel subunits of I_{Kr}, I_{K1}, and possibly I_{Ks}, respectively (90-100). Loss-of-function mutations in I_{Ca,L} subunits would also be expected to decrease APD and promote AF. In 82 patients with Brugada syndrome (which frequently causes AF and produces sudden death due to ventricular fibrillation)/short-QT ECG phenotypes, loss-of-function mutations of the CACNA1C and CACNB2 genes, encoding I_{Ca,L} α- and β-subunits, were associated with AF (Antzelevitch C et al. (2007) Circulation, 115(4): 442-449). Patients with short-QT syndromes have reduced APDs and are predisposed to AF (Mahida S et al. (2011) Cardiovasc Res., 89(4): 692-700).

Several gene variants promote AF by targeting ion channels that govern conduction velocity. The GJA5 gene encodes connexin-40, a gap junction ion channel that is particularly important in the atria. Mice lacking connexin-40 demonstrate conduction abnormalities and atrial arrhythmias (Hagendorff A et al. (1999) Circulation, 99(11); 1508-1515). Missense somatic mutations in GJA5 were identified in 4 of 15 patients with idiopathic AF (Gollob M H et al. (2006) N Engl J Med., 354(25): 2677-2688). GJA5 promoter sequence variants that decrease gene transcription are associated with increased AF vulnerability (Firouzi M et al. (2004) Circ Res., 95(4): e29-e33).

Gene variants impairing Na⁺ channel function also promote AF, presumably via conduction slowing that favors reentry. Loss-of-function Na⁺ channel α-subunit (SCN5A) mutations were first associated with AF in a family with dilated cardiomyopathy, AF, impaired automaticity, and conduction slowing (Olson T M et al. (2005) JAMA, 293(4): 447-454). Subsequently, additional loss-of-function SCN5A mutations and SNPs were identified in idiopathic AF subjects (Chen L Y et al. (2007) Pharmacol Ther., 81(1): 35-41; Ellinor P T et al. (2008) Heart Rhythm., 5(1): 99-105). Loss-of-function SCN5A mutations are the most common genetically defined cause of Brugada syndrome (Francis J and Antzelevitch C (2008) Am Coll Cardiol., 51(12): 1149-1153). More recently, loss-of-function mutations in the cardiac Na⁺ channel β-subunits SCN1B, SCN2B, and SCN3B have been associated with AF (Watanabe H et al. (2010) Circ Arrhythm Electrophysiol., 2(3): 268-275; Olesen M S et al. (2011) Cardiovasc Res., 89(4): 786-793).

AF has been associated with a SNP of the NOS3 gene encoding eNOS (Fatini C et al. (2006) Eur Heart J., 27(14): 1712-1718). Experimental data suggest that eNOS can regulate cardiac vagal activity and I_{Ca,L}, providing plausible links to reentry substrates (Elvan A et al. (1997) J Physiol., 272(1 pt 2): H263-H271; Mery P F et al. (1993) J Biol Chem., 268(35): 26286-26295).

Etiological Contributors to Atrial Fibrillation

A variety of etiological factors contribute to AF occurrence. In most patients, AF results from interactions among multiple factors operating simultaneously.

Over 70% of AF cases have associated heart disease (Kozlowski D et al. (2010) Heart, 96(7): 498-503). Aging is a major risk factor, largely via structural remodeling (Nattel S (2011) Can J Cardiol, 27(1): 19-26; Allessie M A et al. (2001) Circulation, 103(5): 769-777). CHF, hypertension, valvular heart disease, and CAD are common contributors (Allessie M A et al. (2001) Circulation, 103(5): 769-777). Less common predisposing conditions include peri- or myocarditis, atrial myxomas, and hypertrophic cardiomyopathy. Extracardiac conditions also promote AF occurrence. For example, studies suggest that heavy alcohol consumption promotes AF (Mukamal K J et al. (2005) Circulation, 112(12): 1736-1742). In addition, hyperthyroidism is a well-recognized contributor, and the roles of sleep apnea and obesity are increasingly recognized (Auer J et al. (2001) Am Heart, 142(5): 838-842; Schoonderwoerd B A et al. (2008) Europace, 10(1): 668-673). Autonomic tone also is a well-established factor (Chou C C and Chen P S (2009) Cardiol Clin, 27(1): 35-43, viii). Many disease-causing mutations have been identified and their pathophysiology has been analyzed (Mahida S et al. (2011) Cardiovasc Res, 89(4): 692-700).

Heart Failure and Atrial Fibrillation

The reported prevalence of AF in patients with heart failure ranges from 13% to 27% (Middlekauff H R et al. (1991) Circulation, 84: 40-48; Carson P E et al. (1993) Circulation, 87(suppl): VI-102-VI-110; Mahoney P et al. (1999) Am J Cardiol., 83: 1544-1547; Senni M et al. (1998) Circulation, 98: 2282-2289; Deedwania P C et al. (1998) Circulation, 98: 2574-2579). The prevalence of AF in patients with heart failure increases in parallel with the severity of heart disease, ranging from 5% in patients with mild heart failure; 10% to 26% in patients with moderate heart failure; and up to 50% in patients with severe heart failure (Maisel W H et al. (2003) Am J Cardiol., 91: 2D-8D).

Heart failure can increase the risk for the development of AF in several ways, including elevation of cardiac filling pressures, dysregulation of intracellular calcium, and autonomic and neuroendocrine dysfunction. Atrial stretch results in activation of stretch-activated ionic currents, leading to increased dispersion of refractoriness and alterations in anisotropic and conduction properties, facilitating AF (Solti F et al. (1989) Cardiovasc Res., 23: 882-886). Inhibition of these stretch-activated currents by gadolinium can reduce the susceptibility to AF in response to atrial pressure overload (Bode F et al. (2000) Circulation, 101: 2200-2205). Heart failure has been associated with increased interstitial fibrosis, which can lead to abnormal conduction through the atria, creating a substrate for AF in animal models (Li D et al. (1999) Circulation, 100: 87-95; Guerra J M et al. (2006) Circulation, 114: 110-118; Lee K W and Everett T H (2006) Circulation, 114: 1703-1712). Dysregulation of intracellular calcium, an important feature in the pathophysiology of heart failure, also has been found to be associated with AF. The key regulators of intracellular calcium metabolism, the ryanodine receptor and the sarcoplasmic reticulum Ca²⁺-ATPase, are downregulated in AF (Beuckelmann D J et al. (1992) Circulation, 85: 1046-1055; Ohkusa T et al. (1999) J Am Coll Cardiol., 34: 255-263). Heart failure also is characterized by neurohormonal activation, with elevated concentrations of catecholamine and angiotensin II. The degree of neurohormonal activation correlates with the severity of heart failure and promotes structural remodeling and atrial fibrosis, thus altering atrial conduction properties and promoting AF (Li D et al. (1999) Circulation, 100: 87-95; Cha Y M et al. (2003) Am J Physiol Heart Circ Physiol., 284: H1313-H1320).

Extra-Cardiac Factors Contributing to Atrial Fibrillation

Patients with sleep apnea, obesity, diabetes, hypertension, advanced age, valvular, ischemic, and nonischemic structural heart disease are at increased risk of AF (Anter E et al. (2009) Circulation, 119: 2516-2525). For example, sleep apnea increases atrial pressure, causing atrial stretch that could promote remodeling (Schoonderwoerd B A et al. (2008) Europace., 10(1): 668-673; Kannel W B and Benjamin E J (2009) Cardiol Clin., 27(1):13-24, vii). Diabetes causes atrial fibrosis, along with increased expression of the receptor for advanced glycosylation end-products (RAGE) and connective tissue growth factor (CTGF). Suppression of advanced glycosylation endproduct (AGE) production prevented diabetes-related atrial remodeling and CTGF upregulation in a rat model (Kato T et al. (2008) J Cardiovasc Electrophysiol., 19(4): 415-420).

Genetic Factors Contributing to Atrial Fibrillation

A familial occurrence of AF has been recognized, and standard genetic techniques have led to the identification of several chromosomal loci and genes in which mutations can cause dominantly inherited AF (Judge D P (2012) J Am Coll Cardiol., 60(13): 1182-1184). These include genes encoding myocardial potassium (KCNQ1, KCNA5, KCNE5, KCNJ2, and KCNE2) and sodium (SCN5A, SCN1B, SCN2B, and SCN3B) channels, potassium-adenosine triphosphate channels (ABCC9), nucleoporin-155 (NUP155), gap junction protein connexin 40 (GJA5), and atrial natriuretic peptide (NPPA) (Chen Y H et al. (2003) Science, 299: 251-254; Olson T M et al. (2006) Hum Mol Genet., 15: 2185-2191; Raven L S et al. (2008) Heart Rhythm., 5: 427-435; Xia M et al. (2005) Biochem Biophys Res Commun., 332: 1012-1019; Yang Y et al. (2004) Am J Hum Genet., 75: 899-905; Olesen M S et al. (2011) Cardiovasc Res., 89: 786-793; Darbar D et al. (2008) Circulation, 117: 1927-1935; Olson T M et al. (2007) Nat Clin Pract Cardiovasc Med., 4: 110-116; Wirka R C et al. (2011) Circ Arrhythm Electrophysiol., 4: 87-93; Hodgson-Zingman D M et al. (N Engl J Med., 359: 158-165; Watanabe H et al. (2009) Circ Arrhythm Electorphysiol., 2: 268-275). Single-nucleotide polymorphisms (SNPs) on chromosome 4q25 (e.g., rs2200733-T allele) were initially recognized in association with AF as part of genome-wide association studies performed in Iceland (Gudbjartsson D F et al. (2007) Nature, 448: 353-357). This association was replicated in several other cohorts with European ancestry as well as among Han Chinese patients with AF (Gudbjartsson D F et al. (2007) Nature, 448: 353-357; kaab S et al. (2009) Eur Heart J., 30: 813-819).

Postoperative Atrial Fibrillation

Postoperative atrial fibrillation (POAF) is common both after cardiothoracic and noncardiothoracic surgery (Hollenberg S M and Dellinger R P (2000) Critical Care Medicine, 28(10)(Suppl): N145-N150). In patients undergoing cardiothoracic surgery, an incidence of 16-46% has been reported, depending on the extent of postoperative monitoring used and the specific surgical procedures (Hossein G et al. (1997) Annals of Surgery, 226(4): 501-513; Aranki S F et al. (1996) Circulation, 94(3): 390-397; Auer J et al. (2005) Journal of Cardiovascular Surgery, 46(6): 583-588); Auer J et al. (2005) Journal of Cardiovascular Surgery, 20(5): 425-431; Fuller J A et al. (1989) Journal of Thoracic and Cardiovascular Surgery, 97(6): 821-825; Pires L A et al. (1995) American Heart Journal, 129(4): 799-808; Creswell L L et al. (1993) Annals of Thoracic Surgery, 56(3): 539-549; Vaporciyan A A et al. (2004) Journal of Thoracic and Cardiovascular Surgery, 127(3): 779-786). In patients undergoing noncardiothoracic surgery, reported incidence of POAF varies between 0.4% and 12% (Vaporciyan A A et al. (2004) Journal of Thoracic and Cardiovascular Surgery, 127(3): 779-786; Solin G H et al. (2009) Korean Circulation Journal, 39(3): 100-104). POAF can be observed during the entire postoperative course, with a peak between the second and fifth postoperative day (Davis E M et al. (2010) Pharmacotherapy, 30(7): 274e-318e).

Risk of developing POAF may be related to several epidemiological and perioperative predictive factors. General factors include older age, male gender, obesity, preexisting congestive heart failure, chronic renal failure, or COPD (Mathew J P et al. (1996) Journal of the American Medical Association, 276(4): 300-306; Piechowiak M et al. (2006) Thoracic and Cardiovascular Surgeon, 54(4): 259-263). In noncardiothoracic surgery, predictors for POAF are preexisting valvular disease and asthma, intra-abdominal and major vascular procedures, and intraoperative hypotension (Morsi A et al. (1998) Pacing and Clinical Electrophysiology 21(7): 1430-1434).

Although POAF can be self-limiting, it may be associated with hemodynamic derangements, postoperative stroke, perioperative myocardial infarction, ventricular arrhythmias, and heart failure (Echahidid N et al. (2008) Journal of the American College of Cardiology, 51(8): 793-801; Karireviciute D et al. (2009) European Heart Journal, 30(4): 410-425). Development of POAF is associated with a longer hospital stay, greater morbidity and mortality, and increased costs (Davis E M et al. (2010) Pharmacotherapy, 30(7): 274e-318e; Mayson S E et al. (2007) Cardiology in Review, 15(5): 231-241).

Therapeutic Agents Used to Treat Atrial Fibrillation

Class I Anti-Arrhythmic Agents

Class I anti-arrhythmic agents are sodium channel antagonists that act by reducing the rate of rise of Phase 0 of the action potential, thereby slowing conduction. Class I anti-arrhythmic agents are divided into three subclasses: Class Ia; Class Ib and Class Ic.

Class Ia anti-arrhythmic agents are moderately potent sodium channel antagonists which also increase the duration of the action potential and refractory period. Class Ia antiarrhythmic agents include, but are not limited to, quinidine (Quinidex™, Quinaglute™), procainamide (Procan™, Procanbid™) and disopyramide (Norpace™).

Class Ib anti-arrhythmic agents are weak sodium channel antagonists which reduce the duration of the refractory period. Non-limiting examples of such agents include lidocaine (Xylocaine™), mexilitine (Mexitil™), tocainide (Tonocard™) and phenytoin (Dilantin™).

Class Ic anti-arrhythmic agents are strong sodium channel antagonists which slow conduction velocity. Class Ic anti-arrhythmic agents include, but are not limited to, flecainide (Tambocor™), propafenone (Rythmol™) and moricizine.

Class II Anti-Arrhythmic Agents

Class II anti-arrhythmic agents are beta (β) receptor antagonists. These agents block sympathetic nerve activity and reduce conduction velocity. Non-limiting examples of Class II anti-arrhythmic agents include, but are not limited to, acebutolol, atenolol, bisoprolol, esmolol, metoprolol, timolol, nadolol, propanolol, carvedolol and labetalol.

Class III Anti-Arrhythmic Agents

Class III anti-arrhythmic agents primarily block potassium channels, thereby delaying repolarization (Phase 3 of the action potential). These agents act to increase the duration of the action potential and the refractory period. Class III anti-arrhythmic agents include, but are not limited to, bretylium, ibutilide (Corvert™), dofetilide (Tikosyn™), sotalol (Betapace™) and amiodarone (Cordarone™, Pacerone™).

Sotalol prophylaxis has consistently reduced the incidence of postoperative AF (Suttorp M J et al. (1990) J Thorac Cardivasc Surg., 100: 921-926; Suttorp M J et al. (1991) Am J Cardiol., 68: 1163-1169; Nystrom U et al. (1993) Thorac Cardiovasc Surg., 41: 34-37; Pfisterer M E et al. (1997) Ann thorac Surg., 64: 1113-1119; Parikka H et al. (1998) J Cardiovasc Pharmacol., 31: 67-73; Gomes J A et al. (1999) J Am Coll Cardiol., 34: 334-339; Matsuura K et al. (2001) Jpn J Thorac Cardiovasc Surg., 49: 614-617; Forlani S et al. (2002) Ann Thorac Surg., 74: 720-726). Studies have shown that postoperative sotalol prophylaxis may reduce the incidence of AF by 50% or more compared with no sotalol prophylaxis (Suttorp M J et al. (1991) Am J Cardiol., 68: 1163-1169; Parikka H et al. (1998) J Cardiovasc Pharmacol., 31: 67-73; Matsuura K et al. (2001) Jpn J Thorac Cardiovasc Surg., 49: 614-617; Forlani S et al. (2002) Ann Thorac Surg., 74: 720-726). Sotalol may be even more effective when it is initiated before open heart surgery and continued after the operation. When sotalol was initiated up to 48 hours before open heart surgery and was continued after the operation, the incidence of AF was reduced by more than 65% (Nystrom U et al. (1993) Thorac Cardiovasc Surg., 41: 34-37; Gomes J A et al. (1999) J Am Coll Cardiol., 34: 334-339).

In addition to being a potassium channel antagonist, sotalol has been shown to antagonize β-adrenergic receptors (Davis E M et al. (2010) Pharmacotherapy, 30(7): 274e-318e). Studies have shown sotalol to be more effective than conventional β-blockers metoprolol and atenolol (Gold M R et al. (1996) Am J Cardiol., 78: 975-979; Matsuura K et al. (2001) Jpn J Thorac Cardiovasc Surg., 49: 614-617; Forlani S et al. (2002) Ann Thorac Surg., 74: 720-726). Sotalol reduced the incidence of post-operative AF by 10%-34% (Gold M R et al. (1996) Am J Cardiol., 78: 975-979; Matsuura K et al. (2001) Jpn J Thorac Cardiovasc Surg., 49: 614-617; Forlani S et al. (2002) Ann Thorac Surg., 74: 720-726).

Amiodarone is a multichannel blocker possessing a, (3, potassium channel, sodium and calcium-blocking actions that is used to prevent or treat postoperative AF. In most randomized controlled studies, amiodarone was superior to placebo or usual care with absolute reductions in the incidence of postoperative AF between 12% and 51% (Auer J et al. (2004) Am Heart J., 147: 636-643; Barnews B J et al. (2006) Ann Thorac Surg., 82: 1332-1337; Daoud E G et al. (1997) NEJM, 337: 1785-1791; Guarnieri T et al. (1999) J Amer Col Card., 34: 343-347; Giri S et al. (2001) Lancet, 357: 830-836; White D M et al. (2002) Ann Thorac Surg., 74: 69-74; Yazigi A et al. (2002) Cardiothorac Vasc anesth., 16: 603-606; Tokmakoglu H et al. (2002) Eur J Cardiothor Surg., 21: 401-405; White C M et al. (2003) Circulation, 108(Suppl. II): 11200-11206; Mitchell L B et al. (2005) JAMA, 294: 3093-3100; Budeus M et al. (2006) Eur Heart J., 27: 1584-1591; Zebis L R et al. (2007) Ann Thorac Surg., 83: 1326-1331). In one study, a 1-week pre-operative oral regimen of amiodarone decreased the incidence of postoperative AF (25% patients in the amiodarone group vs. 53% patients in the placebo group, p=0.03), postoperative intravenous treatment of amiodarone decreased the incidence of postoperative AF when compared with placebo (35% patients in the amiodarone group vs. 47% patients in the placebo group, p=0.01), and treatment of amiodarone throughout the peri-operative (pre-operative, intra-operative and postoperative phases of surgery) period decreased the incidence of postoperative AF when compared with placebo (16% patients in the amiodarone group vs. 25% patients in the placebo group, p=0.001) (Daoud E G et al. (1997) N Engl J Med., 337: 1785-1791; Guarnieri T et al. (1999) J Am Coll Cardiol., 34: 343-347; Mitchell L B et al. (2005) JAMA, 294: 3093-3100).

Amiodarone has been administered in direct combination with β-blockers (e.g., metoprolol), magnesium, and non-pharmacological treatments such as atrial septal pacing in Bachmann's Bundle for the prevention of postoperative AF (Auer J et al. (2004) Am Heart J., 147: 636-643; Cagli K et al. (2006) J Cardiac Surg., 21: 458-464; White C M et al. (2003) Circulation, 108(Suppl. II): 11200-11206). Amiodarone in direct combination with these pharmacologic and non-pharmacologic options was superior to placebo with absolute reductions in the incidence of postoperative AF by 20%-24% (Auer J et al. (2004) Am Heart J., 147: 636-643; Cagli K et al. (2006) J Cardiac Surg., 21: 458-464; White C M et al. (2003) Circulation, 108(Suppl. II): 11200-11206). The Atrial Fibrillation Suppression Trial (AFIST) I study, in which elderly patients greater than 60 years of age received amiodarone 7 g as a slow-load or 6 g of amiodarone as a fast load in combination with β-blockers, found the incidence of postoperative AF to be significantly lower in amiodarone patients receiving concomitant postoperative β-blockers compared to amiodarone alone (16% versus 35%, p=0.02) (Giri S et al. (2001) Lancet, 357: 830-836).

Dronedarone and KB130015 (KB015), compounds that are structurally related to amiodarone, are thought to possess anti-arrhythmic properties.

Dronedarone [n-(2-butyl-3-(4-(3-dibutylaminopropoxy)-benzoyl)benzofuran-5-yl)-methanesulfonamide] is a benzofuran derivative with structural similarity to amiodarone (Matassini M V et al. (2015) Future Cardiol., 11(6): 705-717). As its main mechanism of action, dronedarone inhibits rapid and slow delayed-rectifier K⁺ currents as well as two-pore K⁺ currents. It delays repolarization and increases atrial action-potential duration, thereby destabilizing AF-maintaining reentry (Matassini M V et al. (2015) Future Cardiol., 11(6): 705-717). In phase III clinical trials, dronedarone lowered the risk of AF relapse by 25% compared with placebo (Singh Bramah N and Connolly S J (2007) N Engl J Med., 357(10): 987-999).

KB130015 (KB015) [2-methyl-3-(3,5-diiodo-4-carboxymethoxybenzyl)benzofuran] has been shown to have anti-arrhythmic properties and to bind to T₃ nuclear receptors (Carlsson B et al. (2002) J Med Chem., 45: 623-630). It acts on multiple ion channels with effects similar to those of Class I, Class III and Class IV anti-arrhythmic agents (Mubagwa K et al. (2003) Cardiovascular Drug Reviews, 21(3): 216-235). In addition, it causes inhibition of G-protein coupled and ATP-sensitive K⁺ currents; and has the ability to slow Na⁺ channel inactivation (Mubagwa K et al. (2003) Cardiovascular Drug Reviews, 21(3): 216-235). KB130015 does not prolong the duration of high-plateau action potentials and thus is devoid of QT lengthening effects (Mubagwa K et al. (2003) Cardiovascular Drug Reviews, 21(3): 216-235).

Class IV Anti-Arrhythmic Agents

Class IV anti-arrhythmic agents act as slow calcium channel antagonists. These agents block L-type calcium channels and are further categorized into dihydropyridines, including, without limitation, amlodipine, isradipine, felodipine, nicardipine, nimodipine and nifedipine. Benzothiazepine calcium channel antagonists, including, but not limited to, diltiazem, and phenylalkylamines such as verapamil and the like, are most effective at slowing atrioventricular (AV) node conduction and to a lesser extent slow sinoatrial (SA) node conduction as well.

Class V Anti-Arrhythmic Agents

Class V anti-arrhythmic agents exert their effects through variable mechanisms. Non-limiting examples include adenosine, digoxin and magnesium.

Adenosine produces acute inhibition of sinus node and atrioventricular (AV) nodal function. This profound but short lived electrophysiologic effect makes adenosine a suitable agent for treating supraventricular tachycardias (SVT) that incorporate the sinus node or AV node as part of the arrhythmia circuit. Its antiadrenergic properties also make it an effective agent for use with some unique atrial and ventricular tachycardias (Wilbur S L and Marchlinski F E (1997) Am J Cardiol., 79(12A): 30-37).

Digoxin is a cardiac glycoside that enhances vagal tone, thereby slowing conduction through the atrioventricular node. It has been studied alone and in combination with β-adrenergic antagonists for the prevention of supraventricular tachycardia (SVT), including postoperative AF, in patients undergoing coronary artery bypass graft surgery (DiDomenico R J and Massad M G (2005) Ann Thorac Surg., 79: 728-740).

Electrolyte deficiencies, including hypomagnesemia, are common complications after open heart surgery (Fuster V et al. (2001) J Am Coll Cardiol., 38: 1231-1266; Chung M K (2000) Crit Care Med., 28: N136-144). It has been hypothesized that prophylactic magnesium supplementation in patients undergoing cardiac operations may decrease the incidence of postoperative arrhythmias, including postoperative AF, and some studies have shown dramatic reductions in the incidence of postoperative AF compared with placebo (Fanning W J et al. (1991) Ann Thorac Surg., 52: 529-533; Nurozler F et al. (1996) J Card Surg., 11: 421-427).

Statins

Statins have pleiotropic (more than one) action, including the modification of atherosclerotic plaques and the improvement of endothelial function. Inflammation plays a role in the pathogenesis of atherosclerosis, and the anti-inflammatory properties of stains likely account for their ability to reduce the incidence of postoperative AF and perioperative cardiovascular events (Main F et al. (2006) Am J Cardiol., 97: 55-60; Patti G et al. (2006) Circulation, 114: 1455-1461). Mariscalco et al. reported that postoperative AF occurred in 29.5% of patients receiving preoperative statin therapy compared with 40.9% of patients who were not receiving such therapy (p=0.021) (Mariscalco G et al. (2007) Ann Thorac Surg., 84: 1158-1164). Non-limiting examples of statins include atorvastatin (Lipitor™) and rosuvastatin (Crestor™).

Anti-Inflammatory Agents

Cardiovascular surgery with cardiopulmonary bypass is associated with a systemic inflammatory response, which may be in part responsible for postoperative AF. Patients with postoperative AF tend to have significantly higher C-reactive protein (CRP), higher white blood cell (WBC) counts, and higher levels of inflammatory cytokines when compared with patients who do not develop postoperative AF (Abdelhadi R H et al. (2004) Am J Cardiol., 93: 1176-1178; Lamm G et al. (2006) J Cardiothorac Vasc Anesth., 20: 51-56). This finding suggests that inflammatory reactions may be important in the pathogenesis of postoperative AF (Abdelhadi R H et al. (2004) Am J Cardiol., 93: 1176-1178; Lamm G et al. (2006) J Cardiothorac Vasc Anesth., 20: 51-56).

Non-steroidal anti-inflammatory drugs (NSAIDs) have the ability to reduce the incidence of postoperative AF. Several studies have shown that the use of NSAIDs reduced the risk of postoperative AF by 46%, 51%, and 41% (Ruffin R T et al. (2008) Curr Med Res Opinion, 24: 1131-1136; Lertsburapa K et al. (2008) J Thor Cardiovasc Surg., 135: 405-411; Mathew J P et al. (2004) JAMA, 291: 1720-1729). Examples of NSAIDs include, but are not limited to, aspirin, celecoxib (Celebrex™), diclofenac (Cambia™, Cataflam™, Voltaren-XR™, Zipsor™, Zorvolex™), diflunisal, etodolac, ibuprofen (Motrin™, Advil™), indomethacin (Indocin™), ketoprofen, ketorolac, nabumetone naproxen (Aleve™, Anaprox™, Naprelan™, Naprosyn™), oxaprozin (Daypro™), piroxicam (Feldene™), salsalate (Disalsate™), sulindac and tolmetin.

Corticosteroids have been traditionally utilized in cardiac surgeries to reduce inflammation to achieve early extubation (removal of endotracheal tube), enhance pulmonary function recovery, or decrease postoperative nausea and vomiting (Davis E M et al. (2010) Pharmacotherapy, 30(7): 274e-318e). Several studies have indicated that the postoperative concentration of CRP was significantly lower in patients who received hydrocortisone than in patients who did not (Omae T and Kanmura Y (2012) J Anesth., 26: 429-437). In nonsurgical patients, corticosteroid treatment has been shown to reduce the incidence of recurrent AF (Omae T and Kanmura Y (2012) J Anesth., 26: 429-437). Examples of corticosteroids include, but are not limited to, cortisone, hydrocortisone, dexamethasone, methylprednisone and prednisone.

Thiazolidinediones (TZDs) may affect postoperative AF through pleiotropic anti-inflammatory activity against macrophage activation and pro-inflammatory cytokines (127; 128). Non-limiting examples include pioglitazone (Actos™) and rosiglitazone (Avandia™).

Omega-3 Fatty Acids

The ability of omega-3 fatty acids to reduce the occurrence of postoperative AF is thought to result from their stabilizing effect on the myocardium, anti-inflammatory properties and possibly antioxidant activity (Korantzopoulos P et al. (2006) J Amer Col Card., 47(2): 467).

Ascorbic Acid

The ability of ascorbic acid (vitamin C) to prevent postoperative AF has been attributed to its antioxidant properties and potential to attenuate inflammation and electrical remodelling (Korantzopoulos P et al. (2005) Int J Cardiol., 102: 321-326).

N-Acetylcysteine

N-acetylcysteine (NAC) has been hypothesized to prevent postoperative AF based on its antioxidant activity as a free-radical scavenger and its ability to reduce cellular damage in the atrium (Canes C A et al. (2007) J Biol Chem., 282: 28063-28073).

Sodium Nitroprusside

Sodium Nitroprusside (SNP) is thought to reduce the incidence of postoperative AF by replenishing nitric oxide function that may be disrupted due to ischemia-reperfusion injury with coronary artery bypass graft surgery. Administration of nitric oxide donors, such as sodium nitroprusside (SNP) could recover this function (Cavolli R et al. (2008) Circulation, 118: 476-481).

Antagonists of Angiotensin II

Angiotensin II has been identified as possibly playing a role in the development of AF in nonsurgical patients (DiDomenico R J and Massad M G (2005) Ann Thorac Surg., 79: 728-740). In one study, the angiotensin-converting-enzyme inhibitor (ACEI) enalapril was associated with a 78% relative risk reduction for the development of AF compared to placebo (Vermes E et al. (2003) Circulation, 107: 2926-2931). In another study, the combination of irbesartan, an angiotensin receptor blocker (ARB), plus amiodarone prevented recurrence of AF more effectively than amiodarone alone (Madrid A H et al. (2002) Circulation, 106: 331-336).

Although effective to treat arrhythmias such as atrial fibrillation, the use of anti-arrhythmic agents is limited by their widespread tissue distribution, long terminal half-lives and numerous and sometimes serious systemic side effects. For example, amiodarone is widely distributed in body tissues, has a terminal half-life ranging from 14-58 days after discontinuation of long-term therapy, and systemic side effects that include pulmonary toxicity (e.g., coughing, painful breathing, shortness of breath), interstitial pneumonitis (inflammation of the mesh-like walls of the alveoli), hypersensitivity pneumonitis (allergic or extrinsic inflammation of the mesh-like walls of the alveoli), blurred vision, visual halos, peripheral neuropathy (e.g., muscle weakness, numbness or tingling in extremities), phototoxicity (chemically-induced skin irritation after exposure to light), changes in thyroid function, hepatic toxicity, pro-arrhythmia, left ventricular dysfunction and hypotension (See, e.g., Sloskey G E (1983) Clin Pharm., 2(4): 330-340; Harris L et al. (1983) Circulation, Vol. 67, No. 1, 45-51).

Therefore, a need exists for a local delivery system comprising a pharmaceutical composition comprising a particulate formulation containing a therapeutic amount of an anti-arrhythmic agent, that when administered, is effective to prevent or reduce the incidence or severity of atrial fibrillation. Because the therapeutic agent is administered locally, its dose is much lower than if administered systemically, so the risk of systemic side effects is lower. That is, the concentration of the therapeutic agent locally, where it exerts its effect, is higher and the plasma concentration is lower than when the therapeutic agent is administered systemically, thus resulting in a localized pharmacological effect at the site of implantation, with less effect in the body. Accordingly, unwanted side effects in the body are less likely to occur.

### SUMMARY

According to one aspect, the described invention provides a method for reducing incidence or severity of atrial fibrillation in a subject at risk thereof, the method comprising (a) providing a delivery system in a form that is malleable comprising a pharmaceutical composition containing a particulate formulation containing a plurality of particles comprising a therapeutic amount of an anti-arrhythmic agent and a pharmaceutically acceptable carrier; (b) administering the malleable delivery system at an implant site in contact with a surface susceptible to atrial fibrillation, wherein (i) the malleable delivery system is effective to contact a surface of a tissue susceptible to atrial fibrillation, adhere to the surface susceptible to atrial fibrillation; conform to contours of the surface susceptible to atrial fibrillation; or a combination thereof; and (ii) release of the therapeutic agent at the implant site may be effective to produce a predominantly localized pharmacologic effect over a desired amount of time, where the desired amount of time is the time necessary to reduce the incidence or severity of atrial fibrillation.

According to another aspect, the described invention provides a malleable drug delivery system for reducing incidence or severity of atrial fibrillation in a subject at risk thereof, comprising (a) a particulate formulation containing a plurality of particles comprising a therapeutic amount of an anti-arrhythmic agent and a pharmaceutically acceptable carrier; the malleable drug delivery system characterized by: (i) its ability to contact a surface of a tissue susceptible to atrial fibrillation, adhere to the surface susceptible to atrial fibrillation; conform to contours of the surface susceptible to atrial fibrillation; or a combination thereof; and (ii) release of the therapeutic agent at the implant site may be effective to produce a predominantly localized pharmacologic effect over a desired amount of time, where the desired amount of time is the time necessary to reduce the incidence or severity of atrial fibrillation.

According to one embodiment, the anti-arrhythmic agent is selected from the group consisting of a Class I anti-arrhythmic agent, a Class II antiarrhythmic agent, a Class III anti-arrhythmic agent, a Class IV anti-arrhythmic agent, a Class V anti-arrhythmic agent and a combination thereof. According to another embodiment, the anti-arrhythmic agent is a Class III anti-arrhythmic agent. According to another embodiment, the Class III anti-arrhythmic agent is amiodarone, a derivative of amiodarone, a metabolite of amiodarone, an analog of amiodarone or a combination thereof. According to anther embodiment, the derivative of amiodarone is dronedarone [n-(2-butyl-3-(4-(3-dibutylaminopropoxy)-benzoyl)benzofuran-5-yl)-methanesulfonamide] or KB130015 (KB015) [2-methyl-3-(3,5-diiodo-4-carboxymethoxybenzyl)benzofuran]. According to another embodiment, the metabolite of amiodarone is mono-N-desethylamiodarone (B2-O-Et-NH-ethyl), di-N-desethylamiodarone (B2-O-Et-NH₂) or (2-butyl-benzofuran-3-yl)-(4-hydroxy-3,5-diiodophenyl)-methanone (B2) carrying an ethanol side chain [(2-butylbenzofuran-3-yl)-[4-(2-hydroxyethoxy)-3,5-diiodophenyl]-methanone (B2-O-Et-OH)]. According to another embodiment, the analog of amiodarone is N-dimethylamiodarone (B2-O-Et-N-dimethyl), N-dipropylamiodarone (B2-O-Et-N-dipropyl), B2-O-carrying an acetate side chain [[4-(2-butyl-benzofuran-3-carbonyl)-2,6-diiodophenyl]-acetic acid; B2-O-acetate], B2-O-Et carrying an propionamide side chain (B2-O-Et-propionamide), or B2-O carrying an ethyl side chain [(2-butylbenzofuran-3-yl)-(4-ethoxy-3,5-diiodophenyl)-methanone; B2-O-Et].

According to one embodiment, the surface susceptible to atrial fibrillation is an atrium, a superior pulmonary vein, a superior vena cava vein, or a coronary sinus.

According to one embodiment, one-half of the therapeutic agent is released from the delivery system at the implant site within 6 hours; within 12 hours, within 1 day, within 2 days, within 3 days, within 4 days, or within 5 days in vivo.

According to one embodiment, the particulate formulation is in form of a filament, a cord, a thread, a string, a film, a sheet or a patch.

According to one embodiment, the therapeutic agent is dispersed throughout the particle, adsorbed onto the particle, or contained in a core surrounded by a coating.

According to one embodiment, a surface of the particle is impregnated with the therapeutic agent. According to another embodiment, the particles comprise a matrix. According to another embodiment, the matrix is impregnated with the therapeutic agent. According to another embodiment, the delivery system is impregnated with the therapeutic agent. According to another embodiment, the therapeutic agent is entrapped by the matrix. According to another embodiment, wherein the therapeutic agent is released from the malleable delivery system into the implant site.

According to one embodiment, the subject at risk of atrial fibrillation is characterized by etiological factors or genetic factors. According to another embodiment, the etiological factors are selected from the group consisting of age, structural remodeling, congestive heart failure (CHF), hypertension, valvular heart disease, coronary artery disease (CAD), peri- or myocarditis, atrial myxomas, hypertrophic cardiomyopathy, alcohol consumption, hyperthyroidism, sleep apnea, obesity and a combination thereof. According to another embodiment, the genetic factors are selected from the group consisting of a gene encoding myocardial potassium (K⁺) channels, a gene encoding sodium (Na⁺) channels, a gene encoding potassium (K⁺)-adenosine triphosphate channels, nucleoporin-155 (NUP155), gap junction protein connexion 40 (GJAS), atrial natriuretic peptide (NPPA), a single-nucleotide polymorphism (SNP) on chromosome 4q25 and a combination thereof. According to another embodiment, the gene encoding myocardial potassium (K⁺) channels are selected from the group consisting of KCNQ1, KCNA5, KCNE5, KCNJ2, KCNE2 and a combination thereof. According to another embodiment, the gene encoding sodium (Na⁺) channels are selected from the group consisting of SCN5A, SCN1B, SCN2B, SCN3B and a combination thereof. According to another embodiment, the gene encoding potassium (K⁺)-adenosine triphosphate channels are ABCC9. According to another embodiment, the SNP on chromosome 4q25 is rs2200733-T allele.

According to one embodiment, the atrial fibrillation occurs postoperatively.

According to one embodiment, the described invention further comprises administering an additional therapeutic agent systemically. According to another embodiment, the additional therapeutic agent is selected from the group consisting of a statin, an anti-inflammatory agent, a thiazolidinedione, an analgesic agent, an anti-infective agent and a combination thereof.

### BRIEF DESCRIPTION OF THE DRAWINGS

FIG. 1 shows a diagram that illustrates electrocardiogram (ECG/EKG) waves and intervals as well as standard time and voltage measures of an ECG/EKG trace. (ECG Learning Center, University of Utah, ecg.utah.edu).

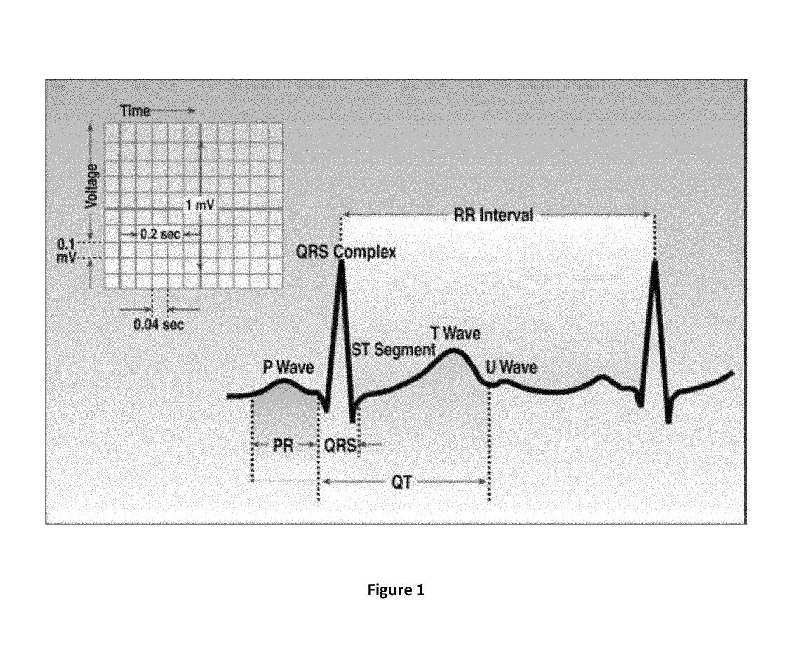

FIG. 2 shows an ECG/EKG trace of atrial fibrillation (top trace) and normal sinus rhythm (bottom trace). Arrows indicate P waves which are lost in atrial fibrillation.

FIG. 3 shows a diagram illustrating the anatomy and cell types of the heart. SAN=sinoatrial node; AVN=atrioventricular node. (Xin M et al. (2013) Nature Reviews Molecular Cell Biology, 14: 529-541).

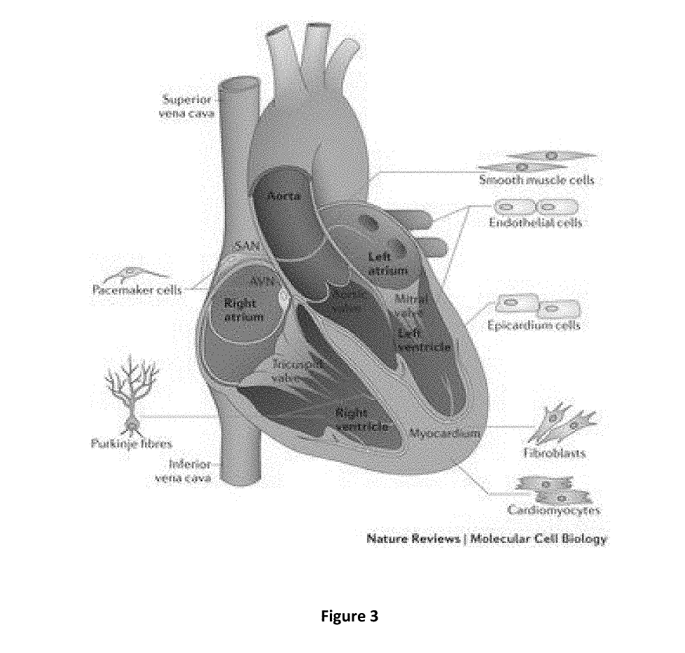

FIG. 4 shows a diagram illustrating the action potential of cardiac muscle cells. TMP=transmembrane potential. (McMaster Pathophysiology Review (MPR), Physiology or Cardiac Conduction and Contractility, Greg Ikonnikov and Deominque Yelle, Editors Eric Wong and Sultan Chaudhry, pathophys.org).

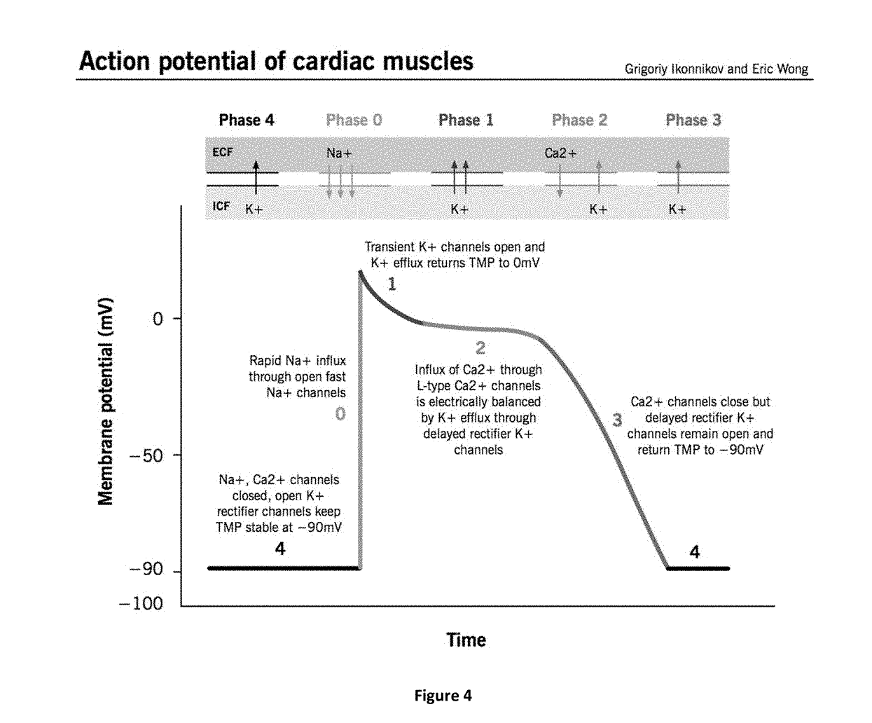

### DETAILED DESCRIPTION OF THE INVENTION

### Glossary

The term “action potential” as used herein refers to an electrical impulse consisting of a self-propagating series of polarizations and depolarizations, transmitted across the plasma membranes of a nerve fiber during transmission of a nerve impulse and across the plasma membranes of a muscle cell during contraction or other activity. In the absence of an impulse, the inside of a cell is electrically negative and the outside of a cell is electrically positive (i.e., resting potential). During passage of an impulse, the inside of a cell becomes electrically positive and the outside of a cell electrically negative.

The term “active” as used herein refers to the ingredient, component or constituent of the composition of the present invention responsible for the intended therapeutic effect. The term “active ingredient” (“AI”), “active pharmaceutical ingredient”, (“API”), or “bulk active” as used herein refers to the substance in a drug that is pharmaceutically active. As used herein, the phrase “additional active ingredient” refers to an agent, other than a compound of the described formulation, that exerts a pharmacological, or any other beneficial activity.

The term “acute” release, as used herein, means delivery of therapeutic levels of an active ingredient for at least 1 hour, 2 hours, 3 hours, 4 hours, 5 hours, 6 hours, 7 hours, 8 hours, 9 hours, 10 hours, 11 hours, or 12 hours.

The term “additive effect”, as used herein, refers to a combined effect of two or more chemicals that is equal to the sum of the effect of each agent given alone.

The term “adhere” as used herein refers to staying attached, clinging to, or to be united by a molecular force acting in the area of contact.

The term “admixture” or “blend” as used herein generally refers to a physical combination of two or more different components.

The term “administer” as used herein means to give or to apply. The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), administered rectally in dosage unit formulations containing conventional nontoxic pharmaceutically acceptable carriers, adjuvants, and vehicles as desired, or administered locally by means such as, but not limited to, injection, implantation, grafting, topical application or parenterally.

The term “agent” as used herein refers generally to compounds that are contained in or on the described formulation. “Agent” includes a single such compound and is also intended to include a plurality of such compounds.

The term “adverse event” (AE), as used herein, refers to any undesirable change from a patient's baseline condition associated with the use of a medical product in a patient. An undesirable change refers to any unfavorable or unintended sign including, but are not limited to, an abnormal laboratory finding, symptom or disease that occurs during the course of a study, whether or not considered related to the study drug, etc. The term “treatment-emergent AE” as used herein refers to any AE temporally associated with the use of a study drug, whether or not considered related to the study drug.

Exemplary adverse events include but are not limited to, any unfavorable and unintended sign including an abnormal laboratory finding, symptom or disease that occurs during the course of the study, whether or not considered related to the study drug; exacerbation of pre-existing disease; increase in frequency or intensity of a pre-existing episodic disease or medical condition; a disease or medical condition detected or diagnosed after study drug administration even though it may have been present prior to the start of the study; continuous persistent disease or symptoms present at baseline that worsen following the start of the study; lack of efficacy in the acute treatment of a life threatening disease; events considered by the investigator to be related to a study mandated procedure; abnormal assessments, e.g., electrocardiographic findings if representing a clinically significant finding not present at baseline or worsened during the course of the study; laboratory test abnormalities if representing a clinically significant finding not present at baseline or worsened during the course of the study or that led to dose reduction, interruption or permanent discontinuation of study drug. Adverse events do not include: a medical or surgical procedure, e.g., surgery, endoscopy, tooth extraction, transfusion; pre-existing disease or a medical condition that does not worsen; or situations in which an adverse change did not occur, e.g., hospitalizations for cosmetic elective surgery.

Adverse events are assessed by the investigators as to whether or not there is a reasonable possibility of causal relationship to the study drug and reported as either related or unrelated. The term “adverse drug reactions related to the study drug” can apply to any adverse event (including serious adverse event) that appears to have a reasonable possibility of a causal relationship to the use of the study drug. The term “adverse drug reactions unrelated to the study drug” applies to any adverse event (including serious adverse event) that does not appear to have a reasonable relationship to the use of the study drug.

The intensity of clinical adverse events is graded on a three-point scale: mild, moderate, severe. If the intensity of an adverse event worsens during study drug administration, only the worst intensity is reported. If the adverse event lessens in intensity, no change in the severity is required. A mild adverse event is one noticeable to subject, but that does not influence daily activities, and usually does not require intervention. A moderate adverse event is one that may make the subject uncomfortable, may influence performance of daily activities, and may require intervention. A severe adverse event is one that may cause noticeable discomfort, usually interferes with daily activities, a result of which a subject may not be able to continue in the study, and for which treatment or intervention is usually needed.

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The term “analog” as used herein refers to a compound having a structure similar to another, but differing from it, for example, in one or more atoms, functional groups, or substructure.

The term “angina pectoris” as used herein refers to a severe constricting chest pain, often radiating from the shoulder to the arm.

The term “angiography” as used herein refers to a technique in which a contrast agent is introduced into the blood stream in order to view blood flow and/or arteries. A contrast agent is required because blood flow and/or arteries sometimes are only weakly apparent in a regular MR scan, CT scan or radiographic film for catheter angiography. Appropriate contrast agents will vary depending upon the imaging technique used. For example, gadolinium is commonly used as a contrast agent used in MR scans. Other MR appropriate contrast agents are known in the art.

The term “antagonist” as used herein refers to a substance that interferes with the effects of another substance. Functional or physiological antagonism occurs when two substances produce opposite effects on the same physiological function. Chemical antagonism or inactivation is a reaction between two substances to neutralize their effects. Dispositional antagonism is the alteration of the disposition of a substance (its absorption, biotransformation, distribution, or excretion) so that less of the agent reaches the target or its persistence there is reduced. Antagonism at the receptor for a substance entails the blockade of the effect of an antagonist with an appropriate antagonist that competes for the same site.

The term “anti-arrhythmic agent” as used herein refers to an agent that prevents or alleviates an abnormal heart rhythm (arrhythmia).

The term “anti-coagulant” as used herein refers to an agent that prevents or alleviates formation of a blood clot.

The term “anti-inflammatory agent” as used herein refers to an agent that prevents or reduces symptoms associated with inflammation.

The term “arrhythmia” as used herein refers to an abnormal heart rhythm. In an arrhythmia, heartbeats may be slow, rapid, irregular or early.

The terms “aseptic manufacturing” and “aseptic processing” as used herein refer to a process by which a final sterile product is realized over several manufacturing process steps. The products/components are sterilized separately and combined later in a sterile environment to produce the final sterile product.

The term “atrial fibrillation” as used herein refers to a rapid, uncoordinated contraction of the atria of the heart resulting in a lack of synchronism between heartbeat and pulse beat. Symptoms of atrial fibrillation include, but are not limited to, palpitations (racing, uncomfortable, irregular heartbeat), weakness, fatigue, reduced ability to exercise, lightheadedness, dizziness, confusion, shortness of breath and chest pain.

The terms “atrial fibrillation ablation” and pulmonary vein isolation” as used herein refer to the process of electrically disconnecting the erratic electrical activity in the pulmonary veins (which can create action potentials at a rate of 400-600 beats per minute) from the rest of the heart, thus effectively eliminating atrial fibrillation. Ablation for atrial fibrillation is complex. It requires multiple catheters and is performed via venous access and puncturing of the interatrial septum to obtain entry to the left atrium where the pulmonary veins empty.

The term “atrium” as used herein refers to one of two (i.e., left atrium and right atrium) blood collection chambers of the heart. The left atrium receives oxygenated blood from the lungs via the pulmonary veins and pumps it into the left ventricle. The right atrium receives deoxygenated blood from the inferior and superior vena cava and pumps it into the pulmonary veins around the lungs.

The term “automaticity” as used herein refers to the capacity of a cell to initiate an impulse without an external stimulus, i.e., the ability of a cell to depolarize, reach threshold potential, and produce a propagated action potential.

The term “bioactive agent” as used herein refers to a compound of interest contained in or on a pharmaceutical formulation or dosage form that is used for pharmaceutical or medicinal purposes to provide a therapeutic effect or elicit a biologic response or activity. “Bioactive agent” includes a single such agent and is also intended to include a plurality of bioactive agents including, for example, combinations of two or more bioactive agents.

The term “bio available” as used herein refers to the rate and extent to which an active ingredient is absorbed from a drug product and becomes available at the site of action.

The term “biocompatible” as used herein refers to that which causes no clinically relevant tissue irritation, injury, toxic reaction, or immunological reaction to living tissue.

The term “biodegradable”, as used herein, refers to a material that will erode to soluble species or that will degrade under physiologic conditions to smaller units or chemical species that are, themselves, non-toxic (biocompatible) to the subject and capable of being metabolized, eliminated, or excreted by the subject.

The term “biomimetic” as used herein refers to materials, substances, devices, processes, or systems that imitate or “mimic” natural materials made by living organisms.

The term “blood vessel”, as used herein, refers to a structure, e.g. a tube or a duct conveying or containing blood. Exemplary blood vessels include, but are not limited to, arteries, arterioles, capillaries, veins and venules.

The term “bradycardia” as used herein refers to a slow arrhythmia (e.g., slower than 60 beats per minute).

The term “carrier” as used herein describes a material that does not cause significant irritation to an organism and does not abrogate the biological activity and properties of an active agent. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “cohesion” and its other grammatical forms as used herein relates to an attractive force between like molecules.

The term “complication” as used herein refers to a pathological process or event during a disorder that is not an essential part of the disease, although it may result from it or from independent causes.

The term “compatible” as used herein refers to components of a composition being capable of being combined with each other in a manner such that there is no interaction that would substantially reduce the efficacy of the composition under ordinary use conditions.

The term “component” as used herein refers to a constituent part, element or ingredient.

The term “composition” as used herein refers to a material formed of two or more substances.

The term “condition”, as used herein refers to a variety of health states and is meant to include disorders or diseases caused by any underlying mechanism, disorder, or injury.

The term “conductivity” as used herein refers to the travel of an electrical impulse from area to another, for example, the ability of one cell to transmit an electrical impulse to another cell.

The term “consequence” as used herein refers to an effect, result or outcome of something that occurred earlier.

The term “consistency” as used herein refers to a degree of density, firmness, thickness or viscosity, etc.

The term “contact” and all its grammatical forms as used herein refers to a state or condition of touching or of immediate or local proximity.

The term “controlled release” as used herein refers to any drug-containing formulation in which the manner and profile of drug release from the formulation are regulated. This refers to immediate as well as non-immediate release formulations, with non-immediate release formulations including, but not limited to, sustained release and delayed release formulations.

The term “cord” as used herein, refers to a structure made of several strands braided, twisted, or woven together.

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

The term “depolarization” as used herein refers to loss of the difference in charge between the inside and the outside of the plasma membrane of a cell due to a change in permeability and migration of sodium ions to the cell interior.

The term “derivative” as used herein means a compound that may be produced from another compound of similar structure in one or more steps. A “derivative” or “derivatives” of a compound retains at least a degree of the desired function of the compound. Accordingly, an alternate term for “derivative” may be “functional derivative.” Derivatives can include chemical modifications, such as akylation, acylation, carbamylation, iodination or any modification that derivatizes the compound. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine. Also included as derivatives or analogs are those peptides that contain one or more naturally occurring amino acid derivative of the twenty standard amino acids, for example, 4-hydroxyproline, 5-hydroxylysine, 3-methylhistidine, homoserine, ornithine or carboxyglutamiate, and can include amino acids that are not linked by peptide bonds. Such peptide derivatives can be incorporated during synthesis of a peptide, or a peptide can be modified by well-known chemical modification methods (see, e.g., Glazer et al., Chemical Modification of Proteins, Selected Methods and Analytical Procedures, Elsevier Biomedical Press, New York (1975)).

The term “diffuse pharmacologic effect”, as used herein, refers to a pharmacologic effect that spreads, disperses or scatters widely over a space or surface.

The term “disease” or “disorder”, as used herein, refers to an impairment of health or a condition of abnormal functioning.

The term “dispersion”, as used herein, refers to a two-phase system, in which one phase is distributed as particles or droplets in the second, or continuous phase. In such systems, the dispersed phase frequently is referred to as the discontinuous or internal phase, and the continuous phase is called the external phase or dispersion medium. For example, in a coarse dispersion, the particle size is 0.5 μm. In a colloidal dispersion, size of the dispersed particle is in the range of approximately 1 nm to 0.5 μm. A molecular dispersion is a dispersion in which the dispersed phase consists of individual molecules; if the molecules are less than colloidal size, the result is a true solution.

The term “disposed”, as used herein, refers to being placed, arranged or distributed in a particular fashion.

The term “drug” as used herein refers to a therapeutic agent or any substance, other than food, used in the prevention, diagnosis, alleviation, treatment or cure of disease.

The term “effective amount” refers to the amount necessary or sufficient to realize a desired biologic effect.

The terms “electrocardiography”, “ECG” and “EKG” as used herein refer to a process of recording the electrical activity of the heart over a period of time using electrodes placed on a patient's body. The electrodes detect small electrical changes on the skin that arise from the heart muscle depolarizing during each heartbeat.

The terms “electrocardiogram”, “ECG trace” and “EKG trace” as used herein refer to the graphic representation of the electrical activity of a patient's heartbeat which comprises three waves: a P wave; a QRS wave complex; and a T wave.

The P wave is a small deflection wave that represents atrial depolarization.

The PR interval is the time between the first deflection of the P wave and the first deflection of the QRS complex.

The QRS wave complex represents ventricular depolarization.

The ST segment, also known as the ST interval, is the time between the end of the QRS complex and the start of the T wave. It reflects the period of zero potential between ventricular depolarization and repolarization.

T waves represent ventricular repolarization (atrial repolarization is obscured by the large QRS complex).

An ECG trace in which there are no P waves and the QRS complexes appear narrow, and at randomly irregular intervals, is the hallmark of atrial fibrillation.

The term “emulsion” as used herein refers to a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil.

The term “excipient” as used herein includes any other agent or compound that may be contained in a long-acting formulation that is not the bioactive agent. As such, an excipient should be pharmaceutically or biologically acceptable or relevant (for example, an excipient should generally be non-toxic to the subject). “Excipient” includes a single such compound and is also intended to include a plurality of such compounds.

The term “excitability” as used herein refers to the ease with which a cell can be depolarized.

The term “fibrillation” as used herein refers to an irregular heart rhythm, for example, an atrial fibrillation or a ventricular fibrillation.

The term “filament” as used herein refers to a very fine thread or threadlike structure, fiber or fibril.

The term “film” as used herein refers to a thin skin or membrane.

The term “flowable”, as used herein, refers to that which is capable of movement in, or as if in, a stream by continuous change of relative position.

The term “fluidity” as used herein refers to the reciprocal of viscosity (1/η).

The term “formulation” as used herein refers to a mixture prepared according to a formula, recipe or procedure.

The terms “functional equivalent” or “functionally equivalent” are used interchangeably herein to refer to substances, molecules, polynucleotides, proteins, peptides, or polypeptides having substantially similar or identical activity or effects.

The term “hydrate” as used herein refers to a compound formed by the addition of water or its elements to another molecule. The water usually can split off by heating, yielding the anhydrous compound.

The term “hydrogel” as used herein refers to a substance resulting in a solid, semisolid, pseudoplastic, or plastic structure containing a necessary aqueous component to produce a gelatinous or jelly-like mass.

The term “hydrophilic” or “hydrophilic agent” as used herein refers to a material or substance having an affinity for polar substances, such as water.

The term “hypersensitivity reaction” as used herein refers to an exaggerated response of the body to a foreign agent. A hypersensitivity reaction can be delayed or immediate. A delayed hypersensitivity reaction is a cell mediated response that occurs in immune individuals peaking at about 24-48 hours after challenge with the same antigen used in an initial challenge. The interaction of T-helper I lymphocytes (Th-I) with MHC Class II positive antigen presenting cells initiates the delayed hypersensitivity reaction. This interaction induces T-helper 1 lymphocytes and macrophages at the site to secrete cytokines. An immediate hypersensitivity reaction is an exaggerated immune response mediated by antibodies occurring within minutes after exposing a sensitized individual to the approximate antigen.

The term “hypertension” as used herein refers to high systemic blood pressure, a transitory or sustained elevation of systemic blood pressure to a level likely to induce cardiovascular damage or other adverse consequences.

The term “hypotension” as used herein refers to subnormal systemic arterial blood pressure; or a reduced pressure or tension of any kind.

The term “implant” as used herein refers to a graft, embedment or insertion of a substance, composition, or device into a pre-determined location within a tissue or space.

The term “impregnate”, as used herein in its various grammatical forms refers to causing to be infused or permeated throughout; or to fill interstices with a substance.

The term “improving patient outcome” as used herein refers to an absence of or diminution of at least one side effect associated with the administration of a therapeutic agent, for example, absence or diminution of hypotension, hypertension, cardiac arrhythmias (e.g., atrial fibrillation), edema, rhabdomyolysis, thrombosis, cerebral infarction or stroke, myocardial infarction or heart attack, or need for blood transfusion.

The phrase “in proximity” as used herein refers to being in the intrapericardial space within less than 10 mm, less than 9.9 mm, less than 9.8 mm, less than 9.7 mm, less than 9.6 mm, less than 9.5 mm, less than 9.4 mm, less than 9.3 mm, less than 9.2 mm, less than 9.1 mm, less than 9.0 mm, less than 8.9 mm, less than 8.8 mm, less than 8.7 mm, less than 8.6 mm, less than 8.5 mm, less than 8.4 mm, less than 8.3 mm, less than 8.2 mm, less than 8.1 mm, less than 8.0 mm, less than 7.9 mm, less than 7.8 mm, less than 7.7 mm, less than 7.6 mm, less than 7.5 mm, less than 7.4 mm, less than 7.3 mm, less than 7.2 mm, less than 7.1 mm, less than 7.0 mm, less than 6.9 mm, less than 6.8 mm, less than 6.7 mm, less than 6.6 mm, less than 6.5 mm, less than 6.4 mm, less than 6.3 mm, less than 6.2 mm, less than 6.1 mm, less than 6.0 mm, less than 5.9 mm, less than 5.8 mm, less than 5.7 mm, less than 5.6 mm, less than 5.5 mm, less than 5.4 mm, less than 5.3 mm, less than 5.2 mm, less than 5.1 mm, less than 5.0 mm, less than 4.9 mm, less than 4.8 mm, less than 4.7 mm, less than 4.6 mm, less than 4.5 mm, less than 4.4 mm, less than 4.3 mm, less than 4.2 mm, less than 4.1 mm, less than 4.0 mm, less than 3.9 mm, less than 3.8 mm, less than 3.7 mm, less than 3.6 mm, less than 3.5 mm, less than 3.4 mm, less than 3.3 mm, less than 3.2 mm, less than 3.1 mm, less than 3.0 mm, less than 2.9 mm, less than 2.8 mm, less than 2.7 mm, less than 2.6 mm, less than 2.5 mm, less than 2.4 mm, less than 2.3 mm, less than 2.2 mm, less than 2.1 mm, less than 2.0 mm, less than 1.9 mm, less than 1.8 mm, less than 1.7 mm, less than 1.6 mm, less than 1.5 mm, less than 1.4 mm, less than 1.3 mm, less than 1.2 mm, less than 1.1 mm, less than 1.0 mm, less than 0.9 mm, less than 0.8 mm, less than 0.7 mm, less than 0.6 mm, less than 0.5 mm, less than 0.4 mm, less than 0.3 mm, less than 0.2 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm, less than 0.01 mm, less than 0.009 mm, less than 0.008 mm, less than 0.007 mm, less than 0.006 mm, less than 0.005 mm, less than 0.004 mm, less than 0.003 mm, less than 0.002 mm less than 0.001 mm from.

The terms “in the body”, “void volume”, “resection pocket”, “excavation”, “injection site”, “deposition site” or “implant site” as used herein are meant to include all tissues of the body without limit, and may refer to spaces formed therein from injections, surgical incisions, tumor or tissue removal, tissue injuries, abscess formation, or any other similar cavity, space, or pocket formed thus by action of clinical assessment, treatment or physiologic response to disease or pathology as non-limiting examples thereof.

The term “infarction” as used herein refers to a sudden insufficiency of arterial or venous blood supply due to emboli, thrombi, mechanical factors, or pressure that produces a macroscopic area of necrosis. The term “infarct” as used herein refers to an area of necrosis resulting from a sudden insufficiency of arterial or venous blood supply.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98% or at least 99%.

The term “inhibitor” as used herein refers to a second molecule that binds to a first molecule thereby decreasing the first molecule's activity. For example, enzyme inhibitors are molecules that bind to enzymes thereby decreasing enzyme activity. The binding of an inhibitor may stop substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “injection”, as used herein, refers to introduction into subcutaneous tissue, or muscular tissue, a vein, an artery or other canals or cavities in the body by force.

The term “injury,” as used herein, refers to damage or harm to a structure or function of the body caused by an outside agent or force, which may be physical or chemical.

The term “ischemia” as used herein refers to a lack of blood supply and oxygen that occurs when reduced perfusion pressure distal to an abnormal narrowing (stenosis) of a blood vessel is not compensated by autoregulatory dilation of the resistance vessels.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95% free of, or more than about 99% free of such components. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “isolated molecule” as used herein refers to a molecule that is substantially pure and is free of other substances with which it is ordinarily found in nature or in vivo systems to an extent practical and appropriate for its intended use.

The term “isomer” as used herein refers to one of two or more molecules having the same number and kind of atoms and hence the same molecular weight, but differing in chemical structure. Isomers may differ in the connectivities of the atoms (structural isomers), or they may have the same atomic connectivities but differ only in the arrangement or configuration of the atoms in space (stereoisomers). Stereoisomers may include, but are not limited to, E/Z double bond isomers, enantiomers, and diastereomers. Structural moieties that, when appropriately substituted, can impart stereoisomerism include, but are not limited to, olefinic, imine or oxime double bonds; tetrahedral carbon, sulfur, nitrogen or phosphorus atoms; and allenic groups. Enantiomers are non-superimposable mirror images. A mixture of equal parts of the optical forms of a compound is known as a racemic mixture or racemate. Diastereomers are stereoisomers that are not mirror images. The invention provides for each pure stereoisomer of any of the compounds described herein. Such stereoisomers may include enantiomers, diastereomers, or E or Z alkene, imine or oxime isomers. The invention also provides for stereoisomeric mixtures, including racemic mixtures, diastereomeric mixtures, or E/Z isomeric mixtures. Stereoisomers can be synthesized in pure form (Nogradi, M.; Stereoselective Synthesis, (1987) VCH Editor Ebel, H. and Asymmetric Synthesis, Volumes 3-5, (1983) Academic Press, Editor Morrison, J.) or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J). In addition the compounds of the described invention may be present as enantiomers, diasteriomers, isomers or two or more of the compounds may be present to form a racemic or diastereomeric mixture.

The term “labile” as used herein refers to that which is subject to increased degradation.

The term “lipophilic” or “lipophilic agent” as used herein refers to a material or substance preferring or possessing an affinity for a non-polar environment compared to a polar or aqueous environment; an agent that is capable of dissolving, of being dissolved in, or of absorbing lipids.

The phrase “localized administration”, as used herein, refers to administration of a therapeutic agent in a particular location in the body.

The phrase “localized pharmacologic effect”, as used herein, refers to a pharmacologic effect limited to a certain location, i.e. in proximity to a certain location, place, area or site. The phrase “predominantly localized pharmacologic effect”, as used herein, refers to a pharmacologic effect of a drug limited to a certain location by at least 1 to 3 orders of magnitude, which is achieved by a localized administration as compared to a systemic administration.

The term “long-term” release, as used herein, refers to delivery of therapeutic levels of the active ingredient for at least about 3 days, and potentially up to about 30 to about 60 days. Terms such as “long-acting”, “sustained-release” or “controlled release” are used generally to describe a formulation, dosage form, device or other type of technologies used, such as, for example, in the art to achieve the prolonged or extended release or bioavailability of a bioactive agent to a subject; it may refer to technologies that provide prolonged or extended release or bioavailability of a bioactive agent to the general systemic circulation or a subject or to local sites of action in a subject including (but not limited to) cells, tissues, organs, joints, regions, and the like. Furthermore, these terms may refer to a technology that is used to prolong or extend the release of a bioactive agent from a formulation or dosage form or they may refer to a technology used to extend or prolong the bioavailability or the pharmacokinetics or the duration of action of a bioactive agent to a subject or they may refer to a technology that is used to extend or prolong the pharmacodynamic effect elicited by a formulation. A “long-acting formulation,” a “sustained release formulation,” or a “controlled release formulation” (and the like) is a pharmaceutical formulation, dosage form, or other technology that is used to provide long-acting release of a bioactive agent to a subject.

Generally, long-acting or sustained release formulations comprise a bioactive agent or agents that is/are incorporated or associated with a biocompatible polymer in one manner or another. The polymers typically used in the preparation of long-acting formulations include, but are not limited, to biodegradable polymers (such as the polyesters poly(lactide), poly(lactide-co-glycolide), poly(caprolactone), poly(hydroxybutyrates), and the like) and non-degradable polymers (such as ethylenevinyl acetate (EVA), silicone polymers and the like). The agent may be blended homogeneously throughout the polymer or polymer matrix or the agent may be distributed unevenly (or discontinuously or heterogeneously) throughout the polymer or polymer matrix (as in the case of a bioactive agent-loaded core that is surrounded by a polymer-rich coating or polymer wall forming material as in the case of a microcapsule, nanocapsule, a coated or encapsulated implant and the like). The dosage form may be in the physical form of particles, film, a fiber, a filament, a sheet, a thread, a cylindrical implant, a asymmetrically-shaped implant or a fibrous mesh (such as a woven or non-woven material; felt; gauze, sponge, and the like). When in the form of particles, the formulation may be in the form of microparticles, nanoparticles, microspheres, nano spheres, microcapsules or nanocapsules, and particles, in general, and combinations thereof. As such, the long-acting (or sustained-release) formulations of the present invention may include any variety of types or designs that are described, used or practiced in the art.

Long-acting formulations containing bioactive agents can be used to achieve local or site-specific delivery to cells, tissues, organs, bones and the like that are located nearby the site of administration. Further, formulations can be used to achieve systemic delivery of the bioactive agent and/or local delivery of the bioactive agent. Formulations can be delivered by injection (through, for example, a needle, a syringe, a trocar, a cannula, and the like) or by implantation. Delivery can be made via any variety of routes of administration commonly used for medical, clinical, surgical purposes including, but not limited to, intravenous, intra-arterial, intramuscular, intraperitoneal, subcutaneous, intradermal, infusion and intracatheter delivery (and the like) in addition to delivery to specific locations (such as local delivery) including stereotactic-guided delivery, infusion delivery, cardiovascular delivery, and any delivery to any multitude of other sites, locations, organs, tissues, etc.

The term “malleable” as used herein refers to being capable of being extended or shaped or molded, or conformable to a diffuse surface.

The term “matrix” as used herein refers to a three dimensional network of fibers that contains voids (or “pores”) where the woven fibers intersect. The structural parameters of the pores, including the pore size, porosity, pore interconnectivity/tortuosity and surface area, affect how substances (e.g., fluid, solutes) move in and out of the matrix.

The terms “minimum effective concentration”, “minimum effective dose,” or “MEC” are used interchangeably to refer to the minimum concentration of an agent required to produce a desired pharmacological effect in most patients. The duration of a drug's action is determined by the time period over which concentrations exceed the MEC. Following administration of a dose of drug, its effects usually show a characteristic temporal pattern. A plot of drug effect vs. time illustrates the temporal characteristics of drug effect and its relationship to the therapeutic window. A lag period is present before the drug concentration exceeds the minimum effective concentration (MEC) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. The therapeutic window reflects a concentration range that provides efficacy without unacceptable toxicity. Accordingly another dose of drug should be given to maintain concentrations within the therapeutic window.

The term “maximum feasible dose” (MFD) as used herein refers to the highest dose of a drug possible based on physical properties that limit the dose formulation concentration; limitations on volume that can be administered, availability of compound or a combination thereof.

The term “maximum tolerated dose” (MTD) as used herein in the context of a toxicity study refers to the highest dose of a drug that does not produce unacceptable toxicity.

The term “metabolite” as used herein refers to any product that remains after a therapeutic agent/compound/drug/composition is broken down by the body.

The term “microparticle composition” or “microparticulate composition”, as used herein, refers to a composition comprising a microparticle formulation and a pharmaceutically acceptable carrier, where the microparticle formulation comprises a therapeutic agent and a plurality of microparticles.

The term “modulate” as used herein means to regulate, alter, adapt, or adjust to a certain measure or proportion.

The term “myocardial infarction” refers to a sudden insufficiency of arterial or venous blood supply to the heart due to emboli, thrombi, mechanical factors, or pressure that produces a macroscopic area of necrosis.

The term “onset” as used herein refers to the start or beginning of something, especially something unpleasant such as a disease or symptoms associated with a disease.

The term “outcome” as used herein refers to a specific result or effect that can be measured.

The term “paste” as used herein refers to a semisoft substance of pliable (meaning flexible, easily bent, deformable) consistency.

The term “parenteral” as used herein refers to introduction into the body by way of an injection (i.e., administration by injection) outside the gastrointestinal tract, including, for example, subcutaneously (i.e., an injection beneath the skin), intramuscularly (i.e., an injection into a muscle); intravenously (i.e., an injection into a vein), intrathecally (i.e., an injection into the subarachnoid space of the spine), intracisternally, intraventricularly, or by infusion techniques. A parenterally administered composition is delivered using a needle, e.g., a surgical needle. The term “surgical needle” as used herein, refers to any needle adapted for delivery of fluid (i.e., those capable of flow) compositions into a selected anatomical structure. Injectable preparations, such as sterile injectable aqueous or oleaginous suspensions, may be formulated according to the known art using suitable dispersing or wetting agents and suspending agents.

The terms “particles”, as used herein, refer to extremely small constituents, e.g., femtoparticles (10⁻¹⁵ m), picoparticles (10⁻¹² m), nanoparticles (10⁻⁹ m), microparticles (10⁻⁶ m), milliparticles (10⁻³ m) that may contain in whole or in part at least one therapeutic agent as described herein. The particles may contain therapeutic agent(s) in a core surrounded by a coating. Therapeutic agent(s) also may be dispersed throughout the particles. Therapeutic agent(s) also may be adsorbed into the particles. The particles may be of any order release kinetics, including zero order release, first order release, second order release, delayed release, sustained release, immediate release, etc., and any combination thereof. The particle may include, in addition to therapeutic agent(s), any of those materials routinely used in the art of pharmacy and medicine, including, but not limited to, erodible, nonerodible, biodegradable, or nonbiodegradable material or combinations thereof. The particles may be microcapsules that contain the voltage-gated calcium channel antagonist in a solution or in a semi-solid state. The particles may be of virtually any shape.

The term “particle size distribution” as used herein refers to the number of particles that fall into each of the various size ranges given as a percentage of the total number of all sizes in the sample of interest. A particle system in which all the particles have the same or almost the same particle size is termed “monodisperse.” A particle system in which the particles are different in size is termed “polydisperse.

The term “perioperative” as used herein refers to the period of time extending from when a patient enters the hospital, clinic or office for surgery until the time the patient is discharged. Peri-operative comprises the preoperative phase (before surgery), the intraoperative phase (during surgery), and postoperative phase.

The term “pharmaceutically acceptable carrier” as used herein refers to one or more compatible solid or liquid filler, diluent or encapsulating substance which is/are suitable for administration to a human or other vertebrate animal. The components of the pharmaceutical compositions also are capable of being commingled in a manner such that there is no interaction which would substantially impair the desired pharmaceutical efficiency.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The term “pharmaceutical formulation” or “pharmaceutical composition” is used herein to refer to a formulation or a composition that is employed to prevent, reduce in intensity, cure or otherwise treat a target condition or disease.

The term “pharmacologic effect” as used herein, refers to a result or consequence of exposure to an active agent.

The term “placebo” as used herein, refers to an inert substance or an inert compound identical in appearance to material being tested in experimental research, in a clinical trial, etc., which is administered to distinguish between drug action and suggestive effect of the material under study.

The term “plasticizer” as used herein refers to a material that, when added to a polymer, imparts an increase in flexibility, workability. And other properties to the finished product. Exemplary plasticizers include, without limitation, phthalic anhydride esters, esters of adipic acid, epoxidized esters, trimellitic esters, triacetin, N-methyl-2-pyrrolidone, glycerol formaldehyde, triethyl citrate (TEC), acetyltributylcitrate, ethanol, and polyethylene glycol.

The term “polymer” as used herein refers to a compound of high molecular weight derived from addition of many smaller molecules (called monomers) linked together or by the condensation of many smaller molecules. The process by which molecules are linked together to form polymers is called “polymerization.” The term “natural polymer” as used herein includes, for example, adhesion proteins, carbohydrates, starches, cellulose, chitosan, chitin, dextrans, collagen, lignans and polyamino acids. The term “synthetic polymer” includes polymers selected from the group consisting of a polyester, a polyethylene glycol polymer, a polyamino-derived biopolymer, a polyanhydride, a polyorthoester, a polyphosphazene, a sucrose acetate isobutyrate (SAIB), a photopolymerizable biopolymer, a polyglycolic acid (PGA), a polylactic acid (PLA); a poly (lactic-co-glycolide) (PLGA), a polycaprolactone; or a copolymer containing polyglycolic acid formed with trimethylene carbonate, polylactic acid (PLA), or polycaprolactone; or a poly d, L (lactic co-caprolactone) copolymer; a copolymer comprising a polyester and polyethylene glycol; or a thermoreversible gel.

The term “premature contraction” as used herein refers to a single heartbeat that occurs earlier than normal.

The term “prevent” as used herein refers to the keeping, hindering or averting of an event, act or action from happening, occurring, or arising.

The term “prodrug” as used herein means a peptide or derivative which is in an inactive form and which is converted to an active form by biological conversion following administration to a subject.

The term “prognosis” as used herein refers to an expected future cause and outcome of a disease or disorder, based on medical knowledge.

The term “pulsatile release” as used herein refers to any drug-containing formulation in which a burst of the drug is released at one or more predetermined time intervals.

The term “recombinant” as used herein refers to a substance produced by genetic engineering.

The term “reduce” or “reducing” as used herein refers to a diminution, a decrease, an attenuation, limitation or abatement of the degree, intensity, extent, size, amount, density, number or occurrence of disorder in individuals at risk of developing the disorder.

The term “refractoriness” as used herein refers to a state of inexcitability that exists during a period of repolarization. The term “absolute refractoriness” as used herein refers to a state of inexcitability due to a membrane potential that is too low to depolarization. The term “relative refractoriness” as used herein refers to a state in which depolarization can be achieved even though repolarization has not fully occurred.

The term “refractory period” as used herein refers to the duration of time after an action potential initiates that a cardiac cell is unable to initiate another action potential. A refractory period lasts approximately 250 milliseconds in duration.

The term “release” and its various grammatical forms, refers to dissolution of an active drug component and diffusion of the dissolved or solubilized species by a combination of the following processes: (1) hydration of a matrix, (2) diffusion of a solution into the matrix; (3) dissolution of the drug; and (4) diffusion of the dissolved drug out of the matrix.

The term “repolarization” as used herein refers to restoration of the difference in charge between the inside of the plasma membrane and the outside of the plasma membrane of a muscle fiber or cell following depolarization.

The term “resting potential” as used herein refers to the electrical potential of a neuron or other excitable cell relative to its surroundings when not stimulated or involved in passage of an impulse.

The term “serious adverse event” as used herein refers to an adverse event that has one or more of the following patient outcomes, or, based on reasonable medical judgment, requires a medical or surgical intervention to prevent one of the following patient outcomes: death, a life-threatening experience, inpatient hospitalization, prolongation of existing hospitalization, a persistent or significant disability or incapacity; a congenital anomaly or birth defect. The term “life-threatening experience” refers to an event in which the subject/patient was at risk of death at the time of the event. It does not refer to an event that hypothetically might have caused death if it were more severe. Important medical events that may not immediately result in death, be life-threatening, or require hospitalization may be considered as a serious adverse event when, based upon appropriate medical judgment, they may jeopardize the patient and may require medical or surgical intervention to prevent one of the outcomes listed in the definitions above. The term “inpatient hospitalization” as used herein refers to an overnight stay in a hospital unit and/or emergency room due to an adverse event. The term “prolongation of existing hospitalization” as used herein refers to at least one overnight stay in the hospital unit and/or emergency room due to the adverse event in addition to the initial inpatient hospitalization. The treatment on an emergency or outpatient basis for an event not fulfilling the definition of seriousness given above and not resulting in hospitalization is not considered a serious adverse event and is reported as an adverse event only. The following reasons for hospitalizations are not considered adverse events or serious adverse events: hospitalizations for cosmetic elective surgery, social and/or convenience reasons; standard monitoring of a pre-existing disease or medical condition that did not worsen, e.g., hospitalization for coronary angiography in a patient with stable angina pectoris; elective treatment of a pre-existing disease or medical condition that did not worsen.

The term “sheet” as used herein, refers to a broad, relatively thin, form, piece or material.

The term “similar” is used interchangeably with the terms analogous, comparable, or resembling, meaning having traits or characteristics in common.

The term “sinoatrial node” or “SA node” as used herein refers to a small body of specialized muscle tissue in the wall of the right atrium of the heart that acts as a pacemaker by producing a contractile signal at regular intervals.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. A “suspension” is a dispersion (mixture) in which a finely-divided species is combined with another species, with the former being so finely divided and mixed that it doesn't rapidly settle out. In everyday life, the most common suspensions are those of solids in liquid. Among the acceptable vehicles and solvents that may be employed are water, Ringer's solution, and isotonic sodium chloride solution.

The term “solvate” as used herein refers to a complex formed by the attachment of solvent molecules to that of a solute. The term “solvent” refers to a substance capable of dissolving another substance (termed a “solute”) to form a uniformly dispersed mixture (solution).

The term “stability” of a pharmaceutical product as used herein refers to the capability of a particular formulation to remain within its physical, chemical, microbiological, therapeutic and toxicological specifications.

The term “statin” as used herein refers to a cholesterol-lowering agent that inhibits the enzyme 3-hydroxy-3-methylglutaryl-coenzyme (HMG-CoA) reductase.

The term “string” as used herein refers to a slender cord or thread.

The term “subacute inflammation” as used herein refers to a tissue reaction typically seen subsequent to the early inflammatory process characterized by a mixture of neutrophils, lymphocytes, and occasionally macrophages and/or plasma cells.

The terms “subject” or “individual” or “patient” are used interchangeably to refer to a member of an animal species of mammalian origin, including humans.

The phrase “subject in need thereof” as used herein refers to a patient that (i) will be administered a formulation containing at least one therapeutic agent, (ii) is receiving a formulation containing at least one therapeutic agent; or (iii) has received a formulation containing at least one therapeutic agent, unless the context and usage of the phrase indicates otherwise.

The term “substantially pure”, as used herein, refers to a condition of a therapeutic agent such that it has been substantially separated from the substances with which it may be associated in living systems or during synthesis. According to some embodiments, a substantially pure therapeutic agent is at least 70% pure, at least 75% pure, at least 80% pure, at least 85% pure, at least 90% pure, at least 95% pure, at least 96% pure, at least 97% pure, at least 98% pure, or at least 99% pure.

The term “suitable for delivery”, as used herein, refers to being apt, appropriate for, designed for, or proper for release only in a site of administration.

The term “susceptible” as used herein refers to a member of a population at risk.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period. Alternatively, delayed absorption of a parenterally administered drug form is accomplished by dissolving or suspending the drug in an oil vehicle. Non-limiting examples of sustained release biodegradable polymers include polyesters, polyester polyethylene glycol copolymers, polyamino-derived biopolymers, polyanhydrides, polyorthoesters, polyphosphazenes, sucrose acetate isobutyrate (SAIB), photopolymerizable biopolymers, protein polymers, collagen, polysaccharides, chitosans, and alginates.

The term “symptom” as used herein refers to a phenomenon that arises from and accompanies a particular disease or disorder and serves as an indication of it.

The term “syndrome,” as used herein, refers to a pattern of symptoms indicative of some disease or condition.

The term “synergistic effect”, as used herein, refers to a combined effect of two active agents, which is greater than the sum of the effects of each agent given alone.

The phrase “systemic administration”, as used herein, refers to administration of a therapeutic agent with a pharmacologic effect on the entire body. Systemic administration includes enteral administration (e.g. oral) through the gastrointestinal tract and parenteral administration (e.g. intravenous, intramuscular, etc.) outside the gastrointestinal tract.

The term “tachycardia” as used herein refers to a rapid arrhythmia (e.g., greater than 100 beats per minute).

The terms “terminal sterilization” and “terminally sterilized” as used herein refer to a process whereby a product is sterilized within its sterile barrier system or whereby the final product is sterilized. The terminal sterilization process is considered a manufacturing process step and usually takes place at, or near, the end of the manufacturing process.

The term “therapeutic agent” as used herein refers to a drug, molecule, nucleic acid, protein, composition or other substance that provides a therapeutic effect. The terms “therapeutic agent” and “active agent” are used interchangeably.

The terms “therapeutic amount”, “therapeutic effective amount” or an “amount effective” of one or more of the therapeutic agents is an amount that is sufficient to provide the intended benefit of treatment. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. A therapeutic effective amount of the therapeutic agents that can be employed ranges from generally 0.1 mg/kg body weight and about 50 mg/kg body weight. A therapeutic effective amount for any particular application may vary depending on such factors as the disease or condition being treated, the particular therapeutic agent being administered, the size of the subject, or the severity of the disease or condition. One of ordinary skill in the art may determine empirically the effective amount of a particular inhibitor and/or other therapeutic agent without necessitating undue experimentation. It is preferred generally that a maximum dose be used, that is, the highest safe dose according to some medical judgment. However, dosage levels are based on a variety of factors, including the type of injury, the age, weight, sex, medical condition of the patient, the severity of the condition, the route of administration, and the particular therapeutic agent employed. Thus the dosage regimen may vary widely, but can be determined routinely by a surgeon using standard methods. “Dose” and “dosage” are used interchangeably herein.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀ which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “thread” as used herein refers to a cord of a material composed of two or more filaments twisted together.

The term “threshold potential” as used herein refers to the critical level to which a membrane potential must be depolarized to initiate an action potential; the transmembrane potential that must be achieved before a membrane channel can open.

The term “topical” as used herein refers to administration of a composition at, or immediately beneath, the point of application. The terms “topical administration” and “topically applying” as used herein are used interchangeably to refer to delivering an active agent onto one or more surfaces of a tissue or cell, including epithelial surfaces. The composition may be applied by pouring, dropping, or spraying, if a liquid; rubbing on, if an ointment, lotion, cream, gel, or the like; dusting, if a powder; spraying, if a liquid or aerosol composition; or by any other appropriate means. Topical administration generally provides a local rather than a systemic effect.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “Vaughn Williams Classification” as used herein refers to a system used to categorize anti-arrhythmic agents based on their mechanism of action. The Vaughn Williams system groups anti-arrhythmic agents into five main classes: Class I agents are fast sodium channel blockers; Class II agents are beta-blockers; Class III agents are potassium channel blockers; Class IV agents are slow calcium channel blockers; and Class V agents are agents of variable mechanisms. Class I agents are further subdivided into three subclasses: Class Ia agents moderately slow conduction and moderately prolong duration of action potential; Class Ib agents minimally slow conduction and shorten duration of action potential; and Class Ic agents markedly slow conduction and cause a minimal duration of action potential.

The term “vehicle” as used herein refers to a substance that facilitates the use of a drug or other material that is mixed with it.

The term “ventricle” as used herein refers to one of two (i.e., left ventricle and right ventricle) large chambers of the heart that collect and expel blood received from an atrium towards peripheral beds within the body and lungs. The left ventricle is located below the left atrium. Oxygenated blood enters the left ventricle from the left atrium. The left ventricle pumps the oxygenated blood out through the aortic valve, into the aortic arch, and onward to the rest of the body. The right ventricle is located below the right atrium and opposite the left ventricle. Deoxygenated blood passes from the right atrium to the right ventricle. The right ventricle pumps the deoxygenated blood through the pulmonary artery and into the lungs.

The term “ventricular fibrillation” as used herein refers to a rapid, uncoordinated fluttering contraction of the ventricle(s) of the heart resulting in loss of synchronization between heartbeat and pulse beat.

The term “ventriculitis”, as used herein refers to inflammation of the ventricles.

The term “viscosity”, as used herein refers to a measure of the tendency of a fluid to resist flow. The resistance is caused by intermolecular friction exerted when layers of fluids attempt to slide by one another. Viscosity can be of two types: dynamic (or absolute) viscosity and kinematic viscosity. Absolute viscosity or the coefficient of absolute viscosity is a measure of the internal resistance. Dynamic (or absolute) viscosity is the tangential force per unit area required to move one horizontal plane with respect to the other at unit velocity when maintained a unit distance apart by the fluid. Dynamic viscosity is usually denoted in poise (P) or centipoise (cP), wherein 1 poise=1 g/cm², and 1 cP=0.01 P.

Kinematic viscosity, a measure of the resistive flow of a fluid under the influence of gravity, is the ratio of absolute or dynamic viscosity to density. It is frequently measured using a capillary viscometer; when two fluids of equal volume are placed in identical capillary viscometers and allowed to flow under the influence of gravity; a viscous liquid takes longer than a less viscous fluid to flow through the tube. Kinematic viscosity is usually denoted in Stoke (St) or Centistokes (cSt), wherein 1 St=10⁻⁴ m²/s, and 1 cSt=0.01 St. Exemplary viscosities are shown in the table below.

| Substance | Temperature (° C.) | η* | |
|---|---|---|---|
| Water | 20 | 1.0020 | mPa s |
| Blood | 37 | 3-4 | mPa s |
| Maple syrup | 20 | 2-3 | Pa s |
| Honey | 20 | 10 | Pa s |
| molasses | 20 | 5 | mPa s |
| Mustard | 25 | 70 | Pa s |
| Olive oil | 20 | 84 | mPa s |
| Peanut butter | 20 | 150-250 | Pa s |
| Hyalgan ® | 20 | 2 | Poise |
| Toothpaste | 20 | 700-1000 | Poise |
| Orthovisc ® | 20 | 1670 | Poise |

*Ten poise (P) equals one pascal second (Pa s), making the centipoise (cP) and millipascal second (mPa s) identical.

Most ordinary liquids have viscosities on the order of 1 to 1000 mPa s, while gases have viscosities on the order of 1 to 10 μPa s. Pastes, gels, emulsions, and other complex liquids are more variable. For example, some are so viscous that they seem more like soft solids than like flowing liquids. Molten glass is extremely viscous and approaches infinite viscosity as it solidifies.

In general, increasing the concentration of a dissolved or dispersed substance generally gives rise to increasing viscosity (that is, thickening), as does increasing the molecular weight of a solute. With Newtonian fluids (typically water and solutions containing only low molecular weight material), viscosity of the fluid is independent of shear strain rate and a plot of shear strain rate (for example, the rate of stirring) against shear stress (for example, force, per unit area stirred, required for stirring) is linear and passes through the origin. Generally the viscosity of a simple liquid decreases with increasing temperature (and vice versa). As temperature increases, the average speed of the molecules in a liquid increases and the amount of time they spend in contact with their nearest neighbors decreases. Thus, as temperature increases, the average intermolecular forces decrease. The exact manner in which the two quantities vary is nonlinear and changes abruptly when the liquid changes phase.

With respect to hydrocolloid solutions (meaning a substance that forms a gel in the presence of water), at moderate concentrations above a critical value (C*), these solutions exhibit non-Newtonian behavior, where their viscosity depends on the shear strain rate, typically as opposite, where γ is the shear strain rate, η0 and η∞ are the viscosities at zero and infinite shear strain rate respectively and τ is a shear-dependent time constant that represents the reciprocal of the shear strain rate required to halve the viscosity. At low flow rates, molecules with preferred conformations that are long and thin have effectively large cross-sections due to them tumbling in solution but at high shear strain rate the molecules align with the flow, giving much smaller effective cross-sections and hence much lower viscosities. More compact molecules are not so much affected by their orientation relative to flow and hence their viscosity changes little with shear strain rate.

In dilute solutions, the relationship of linear and substantially linear polymers depends effectively on the hydrodynamic volume (meaning the volume of a polymer coil or polymer sheet when it is in solution, which can vary depending on how well the polymer interacts with the solvent and the polymer's molecular weight) of the molecules as they freely rotate. The relationship between viscosity with concentration is generally linear up to viscosity values of about twice that of water. This dependency means that more extended molecules increase the viscosity to greater extents at low concentrations than more compact molecules of similar molecular weight. At higher concentrations (above C*) all the polymer molecules in the solution effectively overlap, interpenetrate and become entangled (that is, their total hydrodynamic volume appears greater than the solution volume) even without being stressed, changing the solution behavior from mainly viscous to mainly elastic with the viscosity (η0 at zero stress) governed by the mobility of the polymer molecules. C* will depend on the shear strain rate as, at high shear strain rate, the molecules take up a less voluminous shape. At higher concentrations the viscosity increases to cause apparently synergic behavior of hydrocolloid mixtures.

The term “whole blood” as used herein refers to generally unprocessed or unmodified collected blood containing all of its components, including, but are not limited to, plasma, cellular components (e.g., red blood cells, white blood cells (including lymphocytes, monocytes, eosinophils, basophils, and neutrophils), and platelets), proteins (e.g., fibrinogen, albumin, immunoglobulins), hormones, coagulation factors, and fibrinolytic factors. The term “whole blood” is inclusive of any anticoagulant that may be combined with the blood upon collection.

The term “zero-shear viscosity” as used herein refers to the maximum plateau value of viscosity in an at-rest condition, i.e., as shear stress or shear rate is reduced.

Anatomical Terms

When referring to animals that typically have one end with a head and mouth, with the opposite end often having the anus and tail, the head end is referred to as the cranial end, while the tail end is referred to as the caudal end. Within the head itself, rostral refers to the direction toward the end of the nose, and caudal is used to refer to the tail direction. The surface or side of an animal's body that is normally oriented upwards, away from the pull of gravity, is the dorsal side; the opposite side, typically the one closest to the ground when walking on all legs, swimming or flying, is the ventral side. On the limbs or other appendages, a point closer to the main body is “proximal”; a point farther away is “distal”. Three basic reference planes are used in zoological anatomy. A “sagittal” plane divides the body into left and right portions. The “midsagittal” plane is in the midline, i.e. it would pass through midline structures such as the spine, and all other sagittal planes are parallel to it. A “coronal” plane divides the body into dorsal and ventral portions. A “transverse” plane divides the body into cranial and caudal portions. When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is a Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median. For example, the tip of a human subject's nose is in the median line.

Ipsilateral means on the same side, contralateral means on the other side and bilateral means on both sides. Structures that are close to the center of the body are proximal or central, while ones more distant are distal or peripheral. For example, the hands are at the distal end of the arms, while the shoulders are at the proximal ends.

Delivery System

According to one aspect, the described invention provides a delivery system comprising a pharmaceutical composition comprising a particulate formulation containing a therapeutic amount of a therapeutic agent, that when administered, is effective to prevent or reduce the incidence or severity of atrial fibrillation.

Particulate Formulation

According to some embodiments, a particulate component, comprising a biocompatible polymeric or non-polymeric system, is utilized for producing particles having a therapeutic agent entrapped therein. Following final processing methods, the particles are incorporated into the system and subsequently placed within a delivery apparatus so as to be easily delivered therefrom into an implant site or comparable space, from which the therapeutic agent subsequently is released by drug release mechanism(s).

According to some embodiments, the delivery system of the described invention comprises a particulate composition in a form that is malleable, meaning capable of being extended or shaped or molded, or conformable to a diffuse surface. According to some embodiments, the particulate composition can be provided in form of a filament. According to some embodiments, the particulate composition of the described composition can be provided in form of a cord. According to some embodiments, the particulate composition of the described composition can be provided in form of a thread. According to some embodiments, the particulate composition of the described composition can be provided in form of a string. According to some embodiments, the particulate composition can be provided in form of a film. According to some embodiments, the particulate composition of the described composition can be provided in form of a sheet. According to some embodiments, the form does not disintegrate or fall apart in water.

According to some embodiments, the particulate formulation of the described composition can be provided in form of a spray. According to some embodiments, the spray is a polymeric matrix. Commercially available sprayable polymeric matrices include, without limitation, CoSeal® Surgical Sealant from Baxter Healthcare (Deerfield, Ill.).

According to some embodiments, the particulate formulation of the described composition can be provided in form of a patch. According to some embodiments, the patch is comprised of a bilayer. According to some embodiments, the patch is comprised of a multilayer.

According to some embodiments, the patch is comprised of a controlled-release polymer. According to some embodiments, the polymer is a biodegradable polymer. Biodegradable polymers include, but are not limited to, natural polymers and synthetic polymers. Non-limiting examples of natural polymers include collagen, atelocollagen and gelatin. Non-limiting examples of synthetic polymers include polylactic acid (PLA), polyglycolic acid (PGA), poly(lactic-co-glycolic acid) (PLGA), poly(ethylene glycol) (PEG) 10,000 tetra (4-pentenoate), polycaprolactone, polyparadioxane, polyphosphoesters, polyanhydride, and polyphosphazenes. According to some embodiments, the polymer is a smart polymer. Smart polymers, or environmental responsive polymers, are macromolecules that display physicochemical change in response to environmental stimuli, including, but not limited to, change in temperature, pH, ionic strength, redox potential, biochemical agents, and ultrasound. Advantages of these polymers include, but are not limited to, ease of application, localized delivery of drugs with site-specific action, prolonged delivery period, and decreased systemic drug dosage to minimize the associated side effects. Non-limiting examples of polymers that display thermosensitivity (changes upon temperature change) include poly(N-isopropylacrylamide) (PNIPAAM), poly(ethylene oxide)-poly(propylene oxide)-poly (ethylene oxide) triblock copolymers (PEO-PPO-PEO), poly(ethylene glycol)-poly(lactic acid)-poly(ethylene glycol) triblocks (PEG-PLA-PEG), chitosan grafted with PEG (PEG-g-chitosan) and poly(lactic-co-glycolicacid)-poly(ethylene glycol)-poly(lactic-co-glycolicacid) triblocks (PLGA-PEG-PLGA). Non-limiting examples of pH-sensitive polymeric systems include acrylic acid and methacrylic acid. Polymers that are sensitive to more than one stimulus, for example, pH and temperature, include, but are not limited to PLA/PLGA-PEG diblock, PLA/PLGA-PEG/PLA/PLGA triblock and PLGA-PEG-PLGA triblock.

According to some embodiments, the patch comprises a backing/outer layer (i.e., layer that does not contact a tissue surface or organ surface).

According to some embodiments, the backing/outer layer is comprised of a drug-impermeable material, a liquid-impermeable material or a combination thereof. Backing/outer layer materials include, but are not limited to polyester, polyurethane, polyether, fabrics impregnated with film rendering the fabrics impervious to the drugs and vehicles, regenerated cellulose (e.g., cellophane), acrylonitrile butadiene styrene (ABS) polymer/cellulose acetate, ethyl cellulose, copolymers of plasticized vinylacetate vinylchloride, polyethylene terephthlate, polyethylene, polypropylene, nylon film or nylon fabric impregnated with drug impervious films, polyvinylidene chloride, silicone rubber, natural rubber, polyethylene vinyl acetate, impregnated and coated papers and metallic foils, metalized shaped films of polyvinylchloride (PVC), ABS and other shapeable polymeric sheets or films.

According to some embodiments, the backing/outer layer is comprised of a selectively permeable membrane comprised of a microporous polymer. According to some embodiments, the microporous polymer can be used to exclude transport of compounds having greater than a predetermined molecular weight. Microporous polymers include, without limitation, polycarbonates (i.e., linear polyesters of carbonic acids in which carbonate groups recur in the polymer chain by phosgenation of a dihydroxy aromatic) such as bisphenol A, polyvinylchlorides, polyamides such as polyhexamethylene adipamide and other such polyamides commonly known as “nylon”, modacrylic copolymers such as those formed of polyvinylchloride and acrylonitrile, and styrene-acrylic acid copolymers, polysulfones such as those characterized by diphenylene sulfone groups in a linear chain, halogenated polymers such as polyvinylidene fluoride and polyvinylfluoride, polychloroethers and thermoplastic polyethers, acetal polymers such as polyformaldehyde, acrylic resins such as polyacrylonitrile, polymethyl methacrylate and poly n-butyl methacrylate, polyurethanes, polyimides, polybenzimidazoles, polyvinyl acetate, aromatic and aliphatic polyethers, cellulose esters such as cellulose triacetate, cellulose, collodion, epoxy resins, olefins such as polyethylene and polypropylene, porous rubber, cross-linked poly(ethylene oxide), cross-linked polyvinylpyrrolidone, cross-linked poly(vinyl alcohol); derivatives of polystyrene such as poly (sodium styrenesulfonate) and polyvinylbenzyltrimethyl-ammonium chloride, poly(hydroxyethyl methacrylate), poly(isobutyl vinyl ether), polyisoprenes, polyalkenes, ethylene vinyl acetate copolymers, polyamides, polyurethanes, polyethylene oxides, polyox, polyox blended with polyacrylic acid or Carbopol™, cellulose derivatives such as hydroxypropyl methyl cellulose, hydroxyethyl cellulose, hydroxypropyl cellulose, pectin, starch, guar gum, locust bean gum, and the like.

According to some embodiments, the particulate formulation comprises a plurality of particles. According to some embodiments, the particulate formulation comprises a plurality of milliparticles comprising a therapeutic amount of a first therapeutic agent, wherein the first therapeutic agent is dispersed throughout each milliparticle, adsorbed onto the milliparticles, or is in a core surrounded by a coating. According to some embodiments, the particulate formulation comprises a plurality of microparticles comprising a therapeutic amount of a therapeutic agent, wherein the therapeutic agent is dispersed throughout each microparticle, adsorbed onto the microparticles, or in a core surrounded by a coating. According to some embodiments, the particulate formulation comprises a plurality of nanoparticles comprising a therapeutic amount of a therapeutic agent, wherein the therapeutic agent is dispersed throughout each nanoparticle, adsorbed onto the nanoparticles, or in a core surrounded by a coating. According to some embodiments, the particulate formulation comprises a plurality of picoparticles comprising a therapeutic amount of a therapeutic agent, wherein the therapeutic agent is dispersed throughout each picoparticle, adsorbed onto the picoparticles, or in a core surrounded by a coating. According to some embodiments, the particulate formulation comprises a plurality of femtoparticles comprising a therapeutic amount of a therapeutic agent, wherein the therapeutic agent is dispersed throughout each femtoparticle, adsorbed onto the femtoparticles, or in a core surrounded by a coating.

According to some embodiments, the particles of the particulate formulation are of a distribution of uniform particle size. According to some embodiments, the uniform distribution of particle size is achieved by a homogenization process to form a uniform emulsion comprising, for example, microparticles.

According to some embodiments, the particles comprise a therapeutic agent. According to some embodiments, the therapeutic agent is disposed on or in the particles. According to some embodiments, the therapeutic agent is dispersed throughout the particles. According to some embodiments, a surface of the particles is impregnated with the therapeutic agent. According to some embodiments, the therapeutic agent is adsorbed onto a surface of the particles. According to some embodiments, the therapeutic agent is contained within a core of the particles surrounded by a coating. According to some embodiments, the particles comprise a matrix. According to some embodiments, the matrix comprises the therapeutic agent. According to some embodiments, the matrix is impregnated with the therapeutic agent.

According to some embodiments, the therapeutic agent is an anti-arrhythmic agent. According to some embodiments, the anti-arrhythmic agent is a Class I anti-arrhythmic agent, a Class II anti-arrhythmic agent, a Class III anti-arrhythmic agent, a Class IV anti-arrhythmic agent, a Class V anti-arrhythmic agent, or a combination thereof.

Exemplary Class I anti-arrhythmic agents include, without limitation, quinidine (Quinidex™, Quinaglute™), procainamide (Procan™, Procanbid™) and disopyramide (Norpace™), lidocaine (Xylocaine™) mexilitine (Mexitil™), tocainide (Tonocard™) and phenytoin (Dilantin™), flecainide (Tambocor™), propafenone (Rythmol™) and moricizine.

Exemplary Class II anti-arrhythmic agents include, without limitation, acebutolol, atenolol, bisoprolol, esmolol, metoprolol, timolol, nadolol, propanolol, carvedolol and labetalol.

Exemplary Class III anti-arrhythmic agents include, without limitation, bretylium, ibutilide (Corvert™), dofetilide (Tikosyn™), sotalol (Betapace™) and amiodarone (Cordarone™, Pacerone™).

Class IV anti-arrhythmic agents include, but are not limited to, dihydropyridines, benzothiazepines and phenylalkylamines. Exemplary dihyhdropyridines include amlodipine, isradipine, felodipine, nicardipine, nimodipine and nifedipine. An exemplary benzothiazepine is diltiazem. An exemplary phenylalkylamines includes, without limitation, verapamil.

Exemplary Class V anti-arrhythmic agents include, without limitation, adenosine, digoxin and magnesium.

According to some embodiments, the anti-arrhythmic agent is amiodarone.

According to some embodiments, the anti-arrhythmic agent is a derivative of amiodarone. Derivatives of amiodarone include, but are not limited to, Dronedarone [n-(2-butyl-3-(4-(3-dibutylaminopropoxy)-benzoyl)benzofuran-5-yl)-methanesulfonamide] and KB130015 (KB015) [2-methyl-3-(3,5-diiodo-4-carboxymethoxybenzyl)benzofuran].

According to some embodiments, the anti-arrhythmic agent is a metabolite of amiodarone. Metabolites of amiodarone include, but are not limited, mono-N-desethylamiodarone (B2-O-Et-NH-ethyl), di-N-desethylamiodarone (B2-O-Et-NH₂), and (2-butyl-benzofuran-3-yl)-(4-hydroxy-3,5-diiodophenyl)-methanone (B2) carrying an ethanol side chain [(2-butylbenzofuran-3-yl)-[4-(2-hydroxyethoxy)-3,5-diiodophenyl]-methanone; B2-O-Et-OH].

According to some embodiments, the anti-arrhythmic agent is an analog of amiodarone. Analogs of amiodarone include, but are not limited to, N-dimethylamiodarone (B2-O-Et-N-dimethyl), N-dipropylamiodarone (B2-O-Et-N-dipropyl), B2-O-carrying an acetate side chain [[4-(2-butyl-benzofuran-3-carbonyl)-2,6-diiodophenyl]-acetic acid; B2-O-acetate], B2-O-Et carrying an propionamide side chain (B2-O-Et-propionamide), and B2-0 carrying an ethyl side chain [(2-butylbenzofuran-3-yl)-(4-ethoxy-3,5-diiodophenyl)-methanone; B2-O-Et].

According to some embodiments, the therapeutic agent is an isolated molecule. According to some embodiments, the therapeutic agent is substantially pure.

According to some embodiments, the particles comprise at least one additional therapeutic agent. According to some embodiments, the additional therapeutic agent is disposed on or in the particles. According to some embodiments, the additional therapeutic agent is dispersed throughout the particles. According to some embodiments, a surface of the particles is impregnated with the additional therapeutic agent. According to some embodiments, the additional therapeutic agent is adsorbed onto a surface of the particles. According to some embodiments, the additional therapeutic agent is contained within a core of the particles surrounded by a coating.

According to some embodiments, the additional therapeutic agent is a statin. Non-limiting examples of statins include atorvastatin (Lipitor™) and rosuvastatin (Crestor™). According to some embodiments, the additional therapeutic agent is an omega-3 fatty acid. According to some embodiments, the additional therapeutic agent is ascorbic acid. According to some embodiments, the additional therapeutic agent is N-acetylcysteine (NAC). According to some embodiments, the additional therapeutic agent is sodium nitroprusside (SNP). According to some embodiments, the additional therapeutic agent is an antagonist of angiotensin II. According to some embodiments, the antagonist of angiotensin II is an angiotensin-converting-enzyme (ACE) inhibitor. Exemplary ACE inhibitors include, without limitation, quinapril (Accupril™), perindopril (Aceon™), Ramipril (Altace™), captopril (Capoten™), benazepril (Lotensin™), trandolapril (Mavik™), fosinopril (Monpril™), Lisinopril (Prinivil™, Zestril™), moexipril (Univasc™) and enalapril (Vasotec™). According to some embodiments, the antagonist of angiotensin II is an angiotensin receptor blocker (ARB). Exemplary ARBs include, without limitation, azilsartan (Edarbi™), candesartan (Atacand™), eprosartan (Teveten™), irbesartan (Avapro™), losartan (Cozaar™), olmesartan (Benicar™), telmisartan (Micardis™) and valsartan (Diovan™).

According to some embodiments, the additional therapeutic agent is an anti-inflammatory agent.

According to some embodiments, the anti-inflammatory agent is a steroidal anti-inflammatory agent. The term “steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

According to some embodiments, the anti-inflammatory agent is a nonsteroidal anti-inflammatory agent. The term “non-steroidal anti-inflammatory agent” as used herein refers to a large group of agents that are aspirin-like in their action, including, but not limited to, ibuprofen (Advil™), and naproxen sodium (Aleve™). Additional examples of non-steroidal anti-inflammatory agents include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents.

According to some embodiments, the additional therapeutic agent is a thiazolidinedione (TZD). Non-limiting examples of TZDs include pioglitazone (Actos™) and rosiglitazone (Avandia™).

According to some embodiments, the additional therapeutic agent is an analgesic agent. According to some embodiments, the analgesic agent relieves pain by elevating the pain threshold without disturbing consciousness or altering other sensory modalities. According to some such embodiments, the analgesic agent is a non-opioid analgesic. “Non-opioid analgesics” are natural or synthetic substances that reduce pain but are not opioid analgesics. Examples of non-opioid analgesics include, without limitation, etodolac, indomethacin, sulindac, tolmetin, nabumetone, piroxicam, acetaminophen (Tylenol™), fenoprofen, flurbiprofen, ibuprofen, ketoprofen, naproxen, naproxen sodium, oxaprozin, aspirin, choline magnesium trisalicylate, diflunisal, meclofenamic acid, mefenamic acid, and phenylbutazone. According to some other embodiments, the analgesic is an opioid analgesic. “Opioid analgesics”, “opioid”, or “narcotic analgesics” are natural or synthetic substances that bind to opioid receptors in the central nervous system, producing an agonist action. Examples of opioid analgesics include, without limitation, codeine, fentanyl, hydromorphone, levorphanol, meperidine, methadone, morphine, oxycodone, oxymorphone, propoxyphene, buprenorphine, butorphanol, dezocine, nalbuphine, and pentazocine.

According to some embodiments, the additional therapeutic agent is an anti-infective agent. According to some embodiments, the anti-infective agent is an antibiotic agent. The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, without limitation, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

According to some embodiments, other therapeutic agents, biologically-active agents, drugs, medicaments and inactives may be incorporated into the delivery system for providing a local biological, physiological, or therapeutic effect in the body at various release rates.

According to some embodiments, the particles can be of any order release kinetics, including zero order release, first order release, second order release, delayed release, sustained release, immediate release, and a combination thereof. The particles can include, in addition to therapeutic agent(s), any of those materials routinely used in the art of pharmacy and medicine, including, but not limited to, erodible, nonerodible, biodegradable, or nonbiodegradable material or combinations thereof.

According to some embodiments, the particles are loaded with an average of at least 5% by weight of the therapeutic agent. According to some embodiments, the particles are loaded with an average of at least 10% by weight of the therapeutic agent. According to some embodiments, the particles are loaded with an average of at least 15% by weight of the therapeutic agent. According to some embodiments, the particles are loaded with an average of at least 20% by weight of the therapeutic agent. According to some embodiments, the particles are loaded with an average of at least 25% by weight of the therapeutic agent. According to some embodiments, the particles are loaded with an average of at least 30% by weight of the therapeutic agent. According to some embodiments, the particles are loaded with an average of at least 35% by weight of the therapeutic agent. According to some embodiments, the particles are loaded with an average of at least 40% by weight of the therapeutic agent. According to some embodiments, the particles are loaded with an average of at least 45% by weight of the therapeutic agent. According to some embodiments, the particles are loaded with an average of at least 50% by weight of the therapeutic agent. According to some embodiments, the particles are loaded with an average of at least 55% by weight of the therapeutic agent. According to some embodiments, the particles are loaded with an average of at least 60% by weight of the therapeutic agent. According to some embodiments, the particles are loaded with an average of at least 63% by weight of the therapeutic agent. According to some embodiments, the particles are loaded with an average of at least 65% by weight of the therapeutic agent. According to some embodiments, the particles are loaded with an average of at least 70% by weight of the therapeutic agent. According to some embodiments, the particles are loaded with an average of at least 75% by weight of the therapeutic agent. According to some embodiments, the particles are loaded with an average of at least 80% by weight of the therapeutic agent. According to some embodiments, the particles are loaded with an average of at least 85% by weight of the therapeutic agent. According to some embodiments, the particles are loaded with an average of at least 90% by weight of the therapeutic agent. According to some embodiments, the particles are loaded with an average of at least 95% by weight of the therapeutic agent.

According to some embodiments, the particulate formulation comprises a suspension of particles. According to one embodiment, the particulate formulation comprises a powder suspension of particles. According to some embodiments, the particulate formulation further comprises at least one of a suspending agent, a stabilizing agent and a dispersing agent.

According to some embodiments, the particulate formulation is presented as a solution. According to some embodiments, the particulate formulation is presented as an emulsion.

According to some embodiments, the particulate formulation comprises an aqueous solution of the therapeutic agent in water-soluble form. According to some embodiments, the particulate formulation comprises an oily suspension of the first therapeutic agent. An oily suspension of the first therapeutic agent can be prepared using suitable lipophilic solvents. Exemplary lipophilic solvents or vehicles include, without limitation, fatty oils such as sesame oil, or synthetic fatty acid esters, such as ethyl oleate or triglycerides.

According to some embodiments, the particulate formulation comprises an aqueous suspension of the therapeutic agent. Aqueous injection suspensions, for example, can contain substances which increase the viscosity of the suspension, such as sodium carboxymethyl cellulose, sorbitol, hyaluronic acid, or dextran. Optionally, the suspension can contain suitable stabilizers or agents which increase the solubility of the compounds to allow for the preparation of highly concentrated solutions.

According to some embodiments, the therapeutic agent can be in powder form for constitution with a suitable vehicle, e.g., sterile pyrogen-free water, before use. According to some embodiments, the particulate formulation is dispersed in a vehicle to form a dispersion, with the particles as the dispersed phase, and the vehicle as the dispersion medium.

The particulate formulation can include, for example, microencapsulated dosage forms, and if appropriate, with one or more excipients, encochleated, coated onto microscopic gold particles, contained in liposomes, pellets for implantation into the tissue, or dried onto an object to be rubbed into the tissue. As used herein, the term “microencapsulation” refers to a process in which very tiny droplets or particles are surrounded or coated with a continuous film of biocompatible, biodegradable, polymeric or non-polymeric material to produce solid structures including, but not limited to, nonpareils, pellets, crystals, agglomerates, microspheres, or nanoparticles.

A particulate formulation containing a uniform distribution of microparticle size can be prepared by an emulsion based process. An exemplary microencapsulation process is disclosed and described in U.S. Pat. No. 5,407,609 (entitled Microencapsulation Process and Products Thereof), which is incorporated herein by reference.

According to some embodiments, a process for producing a bioactive agent encapsulated into particles comprises: (a) providing a substantially pure crystalline form of the bioactive agent; (b) adding the substantially pure crystalline form of the bioactive agent to a polymer solution, thereby creating a mixture of the bioactive agent and the polymer solution; (c) homogenizing the mixture to form a disperse phase; (d) mixing the disperse phase with a continuous phase comprising a continuous process medium, thereby forming an emulsion comprising the bioactive agent; (e) forming and extracting the particles comprising the substantially pure bioactive agent; and (f) drying the particles.

It is understood and herein contemplated that where a polymer solution comprises a polymer in an organic solvent forming an oil/water emulsion in the disperse phase, mixing the disperse phase with the continuous phase results in a double emulsion (i.e., a water/oil/water emulsion). Where the polymer solution comprises a polymer in an aqueous solvent such as water, only a single emulsion is formed upon mixing the dispersed phase with the continuous phase.

According to some embodiments, the continuous process medium comprises a surfactant and the bioactive agent saturated with the solvent used in the polymer solution.

The particulate formulation can be in the form of granules, beads, powders, (micro)capsules, emulsions, suspensions, or preparations with protracted release of active compounds, in whose preparation excipients and additives and/or auxiliaries such as disintegrants, binders, coating agents, swelling agents, lubricants, or solubilizers are customarily used as described above. The particulate formulations are suitable for use in a variety of drug delivery systems. See Langer (1990) Science 249, 1527-1533.

Drug Delivery from Bioresorbable Polymers

Site-specific activity generally results if the location in the body into which the formulation is deposited is a fluid-filled space or some type of cavity. This provides high concentrations of the drug at the site where activity is needed, and lower concentrations in the rest of the body, thus decreasing the risk of unwanted systemic side effects.

Site-specific delivery systems, for example, include use of microparticles (of about 1 μm to about 100 μm in diameter), thermoreversible gels (for example, PGA/PEG), and biodegradable polymers (for example, PLA, PLGA) that may be in the form of a film.

The delivery characteristics of the drug and the polymer degradation in vivo also can be modified. For example, polymer conjugation can be used to alter the circulation of the drug in the body and to achieve tissue targeting, reduce irritation and improve drug stability.

Controlled Release Polymeric Drug Delivery Systems

According to some embodiments, the delivery system is a controlled release delivery system. Biodegradable polymeric drug delivery systems that control the release rate of the contained drug in a predetermined manner can overcome practical limitations to targeted delivery. A drug can be attached to soluble macromolecules, such as proteins, polysaccharides, or synthetic polymers via degradable linkages. For example, in animals, antitumor agents such as doxorubicin coupled to N-(2-hydroxypropyl) methacrylamide copolymers showed radically altered pharmacokinetics resulting in reduced toxicity. The half-life of the drug in plasma and the drug levels in the tumor were increased while the concentrations in the periphery decreased. (Kopecek and Duncan, J Controlled Release 6, 315 (1987)). Polymers, such as polyethylene glycol (PEG) can be attached to drugs to either lengthen their lifetime or alter their immunogenicity; drug longevity and immunogenicity also may be affected by biological approaches, including protein engineering and altering glycosylation patterns.

Controlled release systems deliver a drug at a predetermined rate for a definite time period. (Reviewed in Langer, R., “New methods of drug delivery,” Science, 249: 1527-1533 (1990); and Langer, R., “Drug delivery and targeting,” Nature, 392 (Supp.): 5-10 (1998)). Generally, release rates are determined by the design of the system, and are nearly independent of environmental conditions, such as pH. These systems also can deliver drugs for long time periods (days or years). Controlled release systems provide advantages over conventional drug therapies. For example, after ingestion or injection of standard dosage forms, the blood level of the drug rises, peaks and then declines. Since each drug has a therapeutic range above which it is toxic and below which it is ineffective, oscillating drug levels may cause alternating periods of ineffectiveness and toxicity. A controlled release preparation maintains the drug in the desired therapeutic range by a single administration. Other potential advantages of controlled release systems include: (i) localized delivery of the drug to a particular body compartment, thereby lowering the systemic drug level; (ii) preservation of medications that are rapidly destroyed by the body; (iii) reduced need for follow-up care; (iv) increased comfort; and (v) improved compliance. (Langer, R., “New methods of drug delivery,” Science, 249: at 1528).

Optimal control is afforded if the drug is placed in a polymeric material or pump. Polymeric materials generally release drugs by the following mechanisms: (i) diffusion; (ii) chemical reaction, or (iii) solvent activation. The most common release mechanism is diffusion. In this approach, the drug is physically entrapped inside a solid polymer that can then be injected or implanted in the body. The drug then migrates from its initial position in the polymeric system to the polymer's outer surface and then to the body. There are two types of diffusion-controlled systems: reservoirs, in which a drug core is surrounded by a polymer film, which produce near-constant release rates, and matrices, where the drug is uniformly distributed through the polymer system. Drugs also can be released by chemical mechanisms, such as degradation of the polymer, or cleavage of the drug from a polymer backbone. Exposure to a solvent also can activate drug release; for example, the drug may be locked into place by polymer chains, and, upon exposure to environmental fluid, the outer polymer regions begin to swell, allowing the drug to move outward, or water may permeate a drug-polymer system as a result of osmotic pressure, causing pores to form and bringing about drug release. Such solvent-controlled systems have release rates independent of pH. Some polymer systems can be externally activated to release more drug when needed. Release rates from polymer systems can be controlled by the nature of the polymeric material (for example, crystallinity or pore structure for diffusion-controlled systems; the lability of the bonds or the hydrophobicity of the monomers for chemically controlled systems) and the design of the system (for example, thickness and shape). (Langer, R., “New methods of drug delivery,” Science, 249: at 1529).

Polyesters such as lactic acid-glycolic acid copolymers display bulk (homogeneous) erosion, resulting in significant degradation in the matrix interior. To maximize control over release, it is often desirable for a system to degrade only from its surface. For surface-eroding systems, the drug release rate is proportional to the polymer erosion rate, which eliminates the possibility of dose dumping, improving safety; release rates can be controlled by changes in system thickness and total drug content, facilitating device design. Achieving surface erosion requires that the degradation rate on the polymer matrix surface be much faster than the rate of water penetration into the matrix bulk. Theoretically, the polymer should be hydrophobic but should have water-labile linkages connecting monomers. For example, it was proposed that, because of the lability of anhydride linkages, polyanhydrides would be a promising class of polymers. By varying the monomer ratios in polyanhydride copolymers, surface-eroding polymers lasting from 1 week to several years were designed, synthesized and used to deliver nitrosoureas locally to the brain. ((Langer, R., “New methods of drug delivery,” Science, 249: at 1531 citing Rosen et al, Biomaterials 4, 131 (1983); Leong et al, J. Biomed. Mater. Res. 19, 941 (1985); Domb et al, Macromolecules 22, 3200 (1989); Leong et al, J. Biomed. Mater. Res. 20, 51 (1986), Brem et al, Selective Cancer Ther. 5, 55 (1989); Tamargo et al, J. Biomed. Mater. Res. 23, 253 (1989)).

Several different surface-eroding polyorthoester systems have been synthesized. Additives are placed inside the polymer matrix, which causes the surface to degrade at a different rate than the rest of the matrix. Such a degradation pattern can occur because these polymers erode at very different rates, depending on pH, and the additives maintain the matrix bulk at a pH different from that of the surface. By varying the type and amount of additive, release rates can be controlled. ((Langer, R., “New methods of drug delivery,” Science, 249: at 1531 citing Heller, et al, in Biodegradable Polymers as Drug Delivery Systems, M. Chasin and R. Langer, Eds (Dekker, New York, 1990), pp. 121-161)).

According to some embodiments, in order to prolong the effect of a drug, it may be desirable to slow the absorption of the drug. This can be accomplished, for example, by the use of a liquid suspension of crystalline or amorphous material with poor water solubility. The rate of absorption of the drug then depends upon its rate of dissolution which, in turn, may depend upon crystal size and crystalline form. For example, according to some embodiments, a SABER™ Delivery System comprising a high-viscosity base component, is used to provide controlled release of the therapeutic agent. (See U.S. Pat. No. 8,168,217, U.S. Pat. No. 5,747,058 and U.S. Pat. No. 5,968,542, incorporated herein by reference). When the high viscosity SAIB is formulated with drug, biocompatible excipients and other additives, the resulting formulation is liquid enough to inject easily with standard syringes and needles. After injection of a SABER™ formulation, the excipients diffuse away, leaving a viscous depot.

SABER™ formulations comprise a drug and a high viscosity liquid carrier material (HVLCM), meaning nonpolymeric, nonwater soluble liquids with a viscosity of at least 5,000 cP at 37° C. that do not crystallize neat under ambient or physiological conditions. HVLCMs may be carbohydrate-based, and may include one or more cyclic carbohydrates chemically combined with one or more carboxylic acids, such as sucrose acetate isobutyrate (SAIB). HVLCMs also include nonpolymeric esters or mixed esters of one or more carboxylic acids, having a viscosity of at least 5,000 cP at 37° C., that do not crystallize neat under ambient or physiological conditions, wherein when the ester contains an alcohol moiety (e.g., glycerol). The ester may, for example comprise from about 2 to about 20 hydroxy acid moieties.

Additional components can include, without limitation, a rheology modifier, and/or a network former. A rheology modifier is a substance that possesses both a hydrophobic and a hydrophilic moiety used to modify viscosity and flow of a liquid formulation, for example, caprylic/capric triglyceride (Migliol 810), isopropyl myristate (IM or IPM), ethyl oleate, triethyl citrate, dimethyl phthalate, and benzyl benzoate. A network former is a compound that forms a network structure when introduced into a liquid medium. Exemplary network formers include cellulose acetate butyrate, carbohydrate polymers, organic acids of carbohydrate polymers and other polymers, hydrogels, as well as particles such as silicon dioxide, ion exchange resins, and/or fiberglass that are capable of associating, aligning or congealing to form three dimensional networks in an aqueous environment.

According to some embodiments, the particulate composition is effective to release the therapeutic agent(s) over a time period of 12 hours. According to some embodiments, the particulate composition is effective to release the therapeutic agent(s) over a time period of from 12 hours to 4 days, i.e., at least 12 hours, at least 24 hours, at least 36 hours, at least 48 hours, at least 60 hours, at least 72 hours, at least 84 hours, or no more than 4 days, no more than 3 days, no more than 2 days, no more than 1 day. According to some embodiments, the particulate composition is effective to release the therapeutic agent(s) gradually over a time period of 3-5 days (sustained release), i.e., at least 3 days, at least 4 days, or no more than 5 days

According to some embodiments, the controlled release system is capable of releasing one-half of the therapeutic agent from the delivery system at the site of delivery within 6 hours to 5 days in vivo. According to one embodiment, the controlled release system is capable of releasing one-half of the therapeutic agent from the delivery system at the site of delivery within 6 hours. According to one embodiment, the controlled release system is capable of releasing one-half of the therapeutic agent from the delivery system at the site of delivery within 7 hours. According to one embodiment, the controlled release system is capable of releasing one-half of the therapeutic agent from the delivery system at the site of delivery within 8 hours. According to one embodiment, the controlled release system is capable of releasing one-half of the therapeutic agent from the delivery system at the site of delivery within 9 hours. According to one embodiment, the controlled release system is capable of releasing one-half of the therapeutic agent from the delivery system at the site of delivery within 10 hours. According to one embodiment, the controlled release system is capable of releasing one-half of the therapeutic agent from the delivery system at the site of delivery within 11 hours. According to one embodiment, the controlled release system is capable of releasing one-half of the therapeutic agent from the delivery system at the site of delivery within 12 hours. According to one embodiment, the controlled release system is capable of releasing one-half of the therapeutic agent from the delivery system at the site of delivery within 13 hours. According to one embodiment, the controlled release system is capable of releasing one-half of the therapeutic agent from the delivery system at the site of delivery within 14 hours. According to one embodiment, the controlled release system is capable of releasing one-half of the therapeutic agent from the delivery system at the site of delivery within 15 hours. According to one embodiment, the controlled release system is capable of releasing one-half of the therapeutic agent from the delivery system at the site of delivery within 16 hours. According to one embodiment, the controlled release system is capable of releasing one-half of the therapeutic agent from the delivery system at the site of delivery within 17 hours. According to one embodiment, the controlled release system is capable of releasing one-half of the therapeutic agent from the delivery system at the site of delivery within 18 hours. According to one embodiment, the controlled release system is capable of releasing one-half of the therapeutic agent from the delivery system at the site of delivery within 24 hours. According to one embodiment, the controlled release system is capable of releasing one-half of the therapeutic agent from the delivery system at the site of delivery within 36 hours. According to one embodiment, the controlled release system is capable of releasing one-half of the therapeutic agent from the delivery system at the site of delivery within 48 hours. According to one embodiment, the controlled release system is capable of releasing one-half of the therapeutic agent from the delivery system at the site of delivery within 72 hours. According to one embodiment, the controlled release system is capable of releasing one-half of the therapeutic agent from the delivery system at the site of delivery within 90 hours.

According to some embodiments, because the described delivery system is malleable, it can be delivered to an implant site, where it is effective to ensure drug distribution and uniform drug delivery throughout the implant site. According to some embodiments, the delivery system can be manipulated so as to contact a surface of tissue susceptible to arrhythmia. According to some embodiments, the surface of tissue susceptible to arrhythmia comprises an atrium. According to some embodiments, the atrium is a right atrium, a left atrium, or a combination thereof. According to some embodiments, the surface of tissue susceptible to arrhythmia comprises a superior pulmonary vein, a superior vena cava, a coronary sinus or combination thereof. According to some embodiments, the arrhythmia is atrial fibrillation. According to some embodiments, the described delivery system can adhere to the surface susceptible to arrhythmia. According to some embodiments, the described delivery system can conform to contours of the surface. According to some embodiments, the described delivery system can conform to walls, spaces, or voids in the body. According to some embodiments, the described delivery system can fill the spaces or voids.

According to some embodiments, the total dose of the therapeutic agent administered locally is much lower than that administered systemically so the risk of other and unknown side effects is lower. Because the concentration of the therapeutic agent locally where it exerts its effect is higher and the plasma concentration is lower than when the therapeutic agent is administered systemically, this results in a localized pharmacological effect at the site of implantation, with less effect in the body. Accordingly, unwanted side effects in the body are less likely to occur.

According to some embodiments, the release of the therapeutic agent at the site of delivery is effective to produce a predominantly localized pharmacologic effect over a desired amount of time. According to some embodiments, the release of the therapeutic agent at the site of delivery is effective to produce a predominantly localized pharmacologic effect for 1 day. According to some embodiments, the release of the therapeutic agent at the site of delivery is effective to produce a predominantly localized pharmacologic effect for 2 days. According to some embodiments, the release of the therapeutic agent at the site of delivery is effective to produce a predominantly localized pharmacologic effect for 3 days. According to some embodiments, the release of the therapeutic agent at the site of delivery is effective to produce a predominantly localized pharmacologic effect for 4 days. According to some embodiments, the release of the therapeutic agent at the site of delivery is effective to produce a predominantly localized pharmacologic effect for 5 days. According to some embodiments, the release of the therapeutic agent at the site of delivery is effective to produce a predominantly localized pharmacologic effect for 6 days. According to some embodiments, the release of the therapeutic agent at the site of delivery is effective to produce a predominantly localized pharmacologic effect for 7 days.

According to some embodiments, the release of the therapeutic agent at the site of delivery is effective to produce a diffuse pharmacologic effect throughout an implant site over a desired amount of time, where the desired amount of time is the time necessary to prevent or reduce the incidence or severity of atrial fibrillation. According to some embodiments, the release of the therapeutic agent is effective to produce a diffuse pharmacologic effect at the implant site for at least 1 day. According to some embodiments, the release of the therapeutic agent is effective to produce a diffuse pharmacologic effect at the implant site for at least 2 days. According to some embodiments, the release of the therapeutic agent is effective to produce a diffuse pharmacologic effect at the implant site for at least 3 days. According to some embodiments, the release of the therapeutic agent is effective to produce a diffuse pharmacologic effect at the implant site for at least 4 days. According to some embodiments, the release of the therapeutic agent is effective to produce a diffuse pharmacologic effect at the implant site for at least 5 days. According to some embodiments, the release of the therapeutic agent is effective to produce a diffuse pharmacologic effect at the implant site for at least 6 days. According to some embodiments, the release of the therapeutic agent is effective to produce a diffuse pharmacologic effect at the implant site for at least 7 days.

Particle Polymer Matrix

According to some embodiments, the particulate composition comprises a polymer matrix. According to some embodiments, the polymer matrix is a naturally occurring biopolymer matrix, a synthetic polymer matrix, or a combination thereof. According to some embodiments, the polymer is a slow release compound. According to some embodiments, the polymer is a biodegradable polymer. According to some embodiments, the polymer is selected from the group consisting of a polyester, a polyethylene glycol polymer, a polyamino-derived biopolymer, a polyanhydride, a polyorthoester, a polyphosphazene, a sucrose acetate isobutyrate (SAIB), a photopolymerizable biopolymer, a polyglycolic acid (PGA), a polylactic acid (PLA); a polycaprolactone; or a thermoreversible gel. According to some embodiments, the polymer is a copolymer containing polyglycolic acid formed with trimethylene carbonate, polylactic acid (PLA), or polycaprolactone; a poly (lactic-co-glycolide)(PLGA); a poly D, L (lactic co-caprolactone) copolymer; or a copolymer comprising a polyester and polyethylene glycol.

Both non-biodegradable and biodegradable polymeric materials can be used in the manufacture of particles for delivering the therapeutic agents. Such polymers can be natural or synthetic polymers. The polymer is selected based on the period of time over which release is desired. Exemplary bioadhesive polymers include, without limitation, bioerodible hydrogels as described by Sawhney et al in Macromolecules 26: 581-587 (1993), the teachings of which are incorporated herein. According to some embodiments, the bioadhesive polymer is hyaluronic acid. Exemplary bioerodible hydrogels include, but are not limited to, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

According to some embodiments, the polymer enhances aqueous solubility of the particulate formulation. Exemplary polymers include, without limitation, polyethylene glycol, poly-(d-glutamic acid), poly-(1-glutamic acid), poly-(d-aspartic acid), poly-(1-aspartic acid) and copolymers thereof. According to some embodiments, the polyglutamic acids have a molecular weight between about 5,000 to about 100,000, a molecular weight between about 20,000 and about 80,000, or a molecular weight between about 30,000 and about 60,000. According to some embodiments, the polymer is conjugated via an ester linkage to one or more hydroxyls of an inventive epothilone using a protocol as described by U.S. Pat. No. 5,977,163 which is incorporated herein by reference. Exemplary conjugation sites include the hydroxyl off carbon-21 in the case of 21-hydroxy-derivatives. Other conjugation sites include, but are not limited, to the hydroxyl off carbon 3 and/or the hydroxyl off carbon 7.

According to some embodiments, the matrix comprises a polyglycolide (PGA) matrix. PGA is a linear aliphatic polyester developed for use in sutures. Studies have reported PGA copolymers formed with trimethylene carbonate, polylactic acid (PLA), and polycaprolactone. Some of these copolymers may be formulated as microparticles for sustained drug release.

According to some embodiments, the matrix comprises a polyester-polyethylene glycol matrix. Polyester-polyethylene glycol compounds are soft and may be used for drug delivery.

According to some embodiments, the matrix comprises a poly (amino)-derived biopolymer. Exemplary poly (amino)-derived biopolymers include, but are not limited to, those containing lactic acid and lysine as the aliphatic diamine (see, for example, U.S. Pat. No. 5,399,665), and tyrosine-derived polycarbonates and polyacrylates. Modifications of polycarbonates may alter the length of the alkyl chain of the ester (ethyl to octyl), while modifications of polyarylates may further include altering the length of the alkyl chain of the diacid (for example, succinic to sebasic), which allows for a large permutation of polymers and great flexibility in polymer properties.

According to some embodiments, the matrix comprises a polyanhydride matrix. Polyanhydrides are prepared by the dehydration of two diacid molecules by melt polymerization (see, for example, U.S. Pat. No. 4,757,128). These polymers degrade by surface erosion (as compared to polyesters that degrade by bulk erosion). The release of the therapeutic agent can be controlled by the hydrophilicity of the monomers chosen.

According to some embodiments, the matrix comprises a photopolymerizable biopolymer. Exemplary photopolymerizable biopolymers include, without limitation, lactic acid/polyethylene glycol/acrylate copolymers.

According to some embodiments, the matrix comprises a hydrogel. Hydrogels generally comprise a variety of polymers, including hydrophilic polymers, acrylic acid, acrylamide and 2-hydroxyethylmethacrylate (HEMA).

According to some embodiments, the matrix comprises a naturally-occurring biopolymer. Exemplary naturally-occurring biopolymers include, but are not limited to, protein polymers, collagen, polysaccharides, and photopolymerizable compounds.

According to some embodiments, the matrix comprises a protein polymer. Exemplary protein polymers synthesized from self-assembling protein polymers include, for example, silk fibroin, elastin, collagen, and combinations thereof.

According to some embodiments, the matrix comprises a naturally-occurring polysaccharide. Exemplary naturally-occurring polysaccharides include, but are not limited to, chitin and its derivatives, hyaluronic acid, dextran and cellulosic (which generally are not biodegradable without modification), and sucrose acetate isobutyrate (SAIB). Hyaluronic acid (HA), which is composed of alternating glucuronidic and glucosaminidic bonds and is found in mammalian vitreous humor, extracellular matrix of the brain, synovial fluid, umbilical cords and rooster combs from which it is isolated and purified, also can be produced by fermentation processes.

According to some embodiments, the matrix comprises a chitin matrix. Chitin is composed predominantly of 2-acetamido-2-deoxy-D-glucose groups and is found in yeast, fungi and marine invertebrates (shrimp, crustaceous) where it is a principal component of the exoskeleton. Chitin is not water soluble and the deacetylated chitin, chitosan, only is soluble in acidic solutions (such as, for example, acetic acid). Studies have reported chitin derivatives that are water soluble, very high molecular weight (greater than 2 million Daltons), viscoelastic, non-toxic, biocompatible and capable of crosslinking with peroxides, gluteraldehyde, glyoxal and other aldehydes and carbodiamides, to form gels.

Pharmaceutical Carrier

According to some embodiments, the pharmaceutical composition comprises a pharmaceutically acceptable carrier.

According to some embodiments, the pharmaceutically acceptable carrier is a solid carrier or excipient. According to some embodiments, the pharmaceutically acceptable carrier is a gel-phase carrier or excipient. Examples of carriers or excipients include, but are not limited to, calcium carbonate, calcium phosphate, various monomeric and polymeric sugars (including without limitation hyaluronic acid), starches, cellulose derivatives, gelatin, and polymers. An exemplary carrier can also include saline vehicle, e.g. hydroxyl propyl methyl cellulose (HPMC) in phosphate buffered saline (PBS). According to some embodiments, the pharmaceutically acceptable carrier is a buffer solution. Exemplary buffer solutions can include without limitation a phosphate buffered saline (PBS) solution.

According to some embodiments, the pharmaceutically acceptable carrier imparts stickiness to the composition. According to some embodiments, the pharmaceutically acceptable carrier comprises hyaluronic acid.

According to some embodiments, the pharmaceutically acceptable carrier includes, but is not limited to, a gel, slow-release solid or semisolid compound, optionally as a sustained release gel. According to some such embodiments, the therapeutic agent is embedded into the pharmaceutically acceptable carrier. According to some embodiments, the therapeutic agent is coated on the pharmaceutically acceptable carrier. The coating can be of any desired material, preferably a polymer or mixture of different polymers. Optionally, the polymer can be utilized during the granulation stage to form a matrix with the active ingredient so as to obtain a desired release pattern of the active ingredient. The gel, slow-release solid or semisolid compound is capable of releasing the active agent over a desired period of time. The gel, slow-release solid or semisolid compound can be implanted in a tissue for example, but not limited to, in close proximity to a surface susceptible to atrial fibrillation.

According to some embodiments, the pharmaceutically acceptable carrier comprises a slow-release solid compound. According to one such embodiment, the therapeutic agent is embedded in the slow-release solid compound or coated on the slow-release solid compound. According to yet some embodiments, the pharmaceutically acceptable carrier comprises a slow-release particle containing the therapeutic agent.

Exemplary buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

According to some embodiments, the pharmaceutically acceptable carrier is a gel compound, such as a biodegradable hydrogel, e.g., glyceryl monooleate (GMO). Many hydrogels, polymers, hydrocarbon compositions and fatty acid derivatives having similar physical/chemical properties with respect to viscosity/rigidity may function as a semisolid delivery system. According to some embodiments, the hydrogel incorporates and retains significant amounts of water, which eventually will reach an equilibrium content in the presence of an aqueous environment.

According to one embodiment, a GMO hydrogel delivery system can be produced by heating GMO above its melting point (40-50° C.) and by adding a warm aqueous-based buffer or electrolyte solution, such as, for example, phosphate buffer or normal saline, which thus produces a three-dimensional structure. The aqueous-based buffer may be comprised of other aqueous solutions or combinations containing semi-polar solvents.

GMO provides a predominantly lipid-based hydrogel, which has the ability to incorporate lipophilic materials. GMO further provides internal aqueous channels that incorporate and deliver hydrophilic compounds. It is recognized that at room temperature (25° C.), the gel system may exhibit differing phases which comprise a broad range of viscosity measures.

According to some embodiments, two gel system phases are utilized due to their properties at room temperature and physiologic temperature (about 37° C.) and pH (about 7.4). Within the two gel system phases, the first phase is a lamellar phase of approximately 5% to approximately 15% H₂O content and approximately 95% to approximately 85% GMO content. The lamellar phase is a moderately viscous fluid that may be easily manipulated, poured and injected. The second phase is a cubic phase consisting of approximately 15% to approximately 40% H₂O content and approximately 85%-60% GMO content. It has an equilibrium water content of approximately 35% to approximately 40% by weight. The term “equilibrium water content” as used herein refers to maximum water content in the presence of excess water. Thus the cubic phase incorporates water at approximately 35% to approximately 40% by weight. The cubic phase is highly viscous. The viscosity exceeds 1.2 million centipoise (cp) when measured by a Brookfield viscometer; where 1.2 million cp is the maximum measure of viscosity obtainable via the cup and bob configuration of the Brookfield viscometer.

Alternatively, according to some embodiments, modified formulations and methods of production are utilized such that the nature of the delivery system is altered, or in the alternative, aqueous channels contained within the delivery system are altered. Thus, various therapeutic agents in varying concentrations may diffuse from the delivery system at differing rates, or be released therefrom over time via the aqueous channels of the delivery system. Hydrophilic substances may be utilized to alter the consistency or therapeutic agent release by alteration of viscosity, fluidity, surface tension or the polarity of the aqueous component. For example, glyceryl monostearate (GMS), which is structurally identical to GMO with the exception of a double bond at Carbon 9 and Carbon 10 of the fatty acid moiety rather than a single bond, does not gel upon heating and the addition of an aqueous component, as does GMO. However, because GMS is a surfactant, GMS is miscible in water up to approximately 20% weight/weight. The term “surfactant” as used herein refers to a surface active agent that is miscible in water in limited concentrations as well as polar substances. Upon heating and stirring, the 80% H₂O/20% GMS combination produces a spreadable paste having a consistency resembling hand lotion. The paste then is combined with melted GMO so as to form the cubic phase gel possessing a high viscosity referred to above.

According to some embodiments, a hydrolyzed gelatin, such as commercially available Gelfoam™, can be utilized for altering the aqueous component. Approximately 6.25% to 12.50% concentration of Gelfoam™ by weight may be placed in approximately 93.75% to 87.50% respectively by weight H₂O or another aqueous based buffer. Upon heating and stirring, the H₂O (or other aqueous buffer)/Gelfoam™ combination produces a thick gelatinous substance. The resulting substance is combined with GMO; a product so formed swells and forms a highly viscous, translucent gel being less malleable in comparison to neat GMO gel alone.

According to some embodiments, polyethylene glycols (PEG's) can be utilized for altering the aqueous component to aid in drug solubilization. Approximately 0.5% to 40% concentration of PEG's (depending on PEG molecular weight) by weight can be placed in approximately 99.5% to 60% H₂O or other aqueous based buffer by weight. Upon heating and stirring, the H₂O (or other aqueous buffer)/PEG combination produces a viscous liquid to a semisolid substance. The resulting substance is combined with GMO, whereby a product so formed swells and forms a highly viscous gel.

According to some embodiments, the therapeutic agent releases from the delivery system through diffusion, conceivably in a biphasic manner. A first phase may involve, for example, a lipophilic drug contained within the lipophilic membrane that diffuses therefrom into the aqueous channel. The second phase may involve diffusion of the drug from the aqueous channel into the external environment. Being lipophilic, the drug may orient itself inside the GMO gel within its proposed lipid bi-layer structure. Thus, incorporating greater than approximately 7.5% of the drug by weight into GMO causes a loss of the integrity of the three-dimensional structure whereby the gel system no longer maintains the semisolid cubic phase, and reverts to the viscous lamellar phase liquid. According to some embodiments, about 1% to about 45% of therapeutic agent is incorporated by weight into a GMO gel at physiologic temperature without disruption of the normal three-dimensional structure. As a result, this system can allow for increased flexibility with drug dosages.

Alternatively, the described invention may provide a delivery system, which acts as a vehicle for local delivery of therapeutic agents, comprising a lipophilic, hydrophilic or amphophilic, solid or semisolid substance, heated above its melting point and thereafter followed by inclusion of a warm aqueous component so as to produce a gelatinous composition of variable viscosity based on water content. Therapeutic agent(s) is/are incorporated and dispersed into the melted lipophilic component or the aqueous buffer component prior to mixing and formation of the semisolid system. The gelatinous composition is placed within the semisolid delivery apparatus for subsequent placement, or deposition. Being malleable, the gel system is easily delivered and manipulated via the semisolid delivery apparatus in an implant site, where it can adhere, conform, or a combination thereof, to contours of the implantation site, spaces, or other voids in the body as well as completely filling all voids existing.

According to some embodiments, the pharmaceutical composition further comprises a preservative agent. According to some such embodiments, the pharmaceutical composition is presented in a unit dosage form. Exemplary unit dosage forms include, but are not limited to, ampoules or multi-dose containers.

According to some embodiments, the pharmaceutical composition may further comprise an adjuvant. Exemplary adjuvants include, but are not limited to, preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. Isotonic agents, for example, sugars, sodium chloride and the like, can also be included. Prolonged absorption of an injectable pharmaceutical form can be brought about by the use of agents delaying absorption, for example, aluminum monostearate and gelatin.

Formulations of the delivery system can be sterilized, for example, by terminal gamma irradiation, filtration through a bacterial-retaining filter or by incorporating sterilizing agents in the form of sterile solid compositions that may be dissolved or dispersed in sterile water or other sterile injectable medium just prior to use.

Methods

According to another aspect, the described invention provides a method for reducing incidence or severity of atrial fibrillation, the method comprising providing a delivery system in a malleable form comprising a pharmaceutical composition containing a particulate formulation containing a plurality of particles comprising a therapeutic amount of a therapeutic agent and a pharmaceutically acceptable carrier; wherein the therapeutic amount of the therapeutic agent is effective to reduce the incidence or severity of atrial fibrillation. According to some embodiments, the delivery system is formulated for delivery in the body locally to an implant site. According to some embodiments, because it is malleable, it is effective to contact a surface of tissue susceptible to atrial fibrillation, adhere to the surface susceptible to atrial fibrillation; conform to contours of the surface susceptible to atrial fibrillation; or a combination thereof. According to some embodiments, the delivery system is capable of releasing one-half of the therapeutic agent from the delivery system at the site of delivery within 12 hours; within 12 hours to 4 days; or within 3 to 5 days in vivo. According to some embodiments, release of the therapeutic agent at the site of delivery is effective to produce a predominantly localized pharmacologic effect over a desired amount of time, where the desired amount of time is the time necessary to reduce the incidence or severity of atrial fibrillation. According to some embodiments, the method comprises placing the delivery system in an implant site in proximity to a surface susceptible to atrial fibrillation.

According to some embodiments, the atrial fibrillation occurs postoperatively or as a consequence of cardiac surgery in a subject at risk thereof.

According to some embodiments, the atrial fibrillation occurs as a consequence of out-of-hospital cardiac arrest in a subject at risk thereof.

According to some embodiments, the subject at risk is characterized by etiological factors including without limitation, age, structural remodeling, congestive heart failure (CHF), hypertension, valvular heart disease, coronary artery disease (CAD), peri- or myocarditis, atrial myxomas, hypertrophic cardiomyopathy, heavy alcohol consumption, hyperthyroidism, sleep apnea and obesity.

According to some embodiments, the subject at risk is further characterized by genetic factors including genes encoding myocardial potassium (KCNQ1, KCNA5, KCNE5, KCNJ2, and KCNE2) and sodium (SCN5A, SCN1B, SCN2B, and SCN3B) channels, potassium-adenosine triphosphate channels (ABCC9), nucleoporin-155 (NUP155), gap junction protein connexin 40 (GJA5), atrial natriuretic peptide (NPPA) and single-nucleotide polymorphisms (SNPs) on chromosome 4q25 (e.g., rs2200733-T allele).

According to some embodiments, the surface of tissue susceptible to atrial fibrillation is a left atrium, a right atrium, a superior pulmonary vein, a superior vena cava, a coronary sinus or a combination thereof.

According to some embodiments, the subject in need thereof experiences symptoms of atrial fibrillation. Non-limiting examples of symptoms associated with atrial fibrillation include palpitations (racing, uncomfortable, irregular heartbeat), weakness, fatigue, reduced ability to exercise, lightheadedness, dizziness, confusion, shortness of breath and chest pain. According to some embodiments, a therapeutic amount of the pharmaceutical composition is effective to reduce incidence or severity of atrial fibrillation as determined by the absence or alleviation of one or more symptoms associated with atrial fibrillation. According to some embodiments, reduction of the incidence or severity of atrial fibrillation can be determined by electrocardiogram (ECG/EKG).

For example, a standard 12-lead electrocardiogram (ECG/EKG) represents the heart's electrical activity recorded over a period of time using electrodes placed on the surface of a patient's body. The leads are designated Leads I-III; Leads aVR, aVL and aVF; and Leads V1-V6. Lead I is oriented from right arm (− pole) to left arm (+ pole); Lead II is oriented from right arm (− pole) to left leg (+ pole); and Lead III is oriented from left arm (− pole) to left leg (+ pole). Lead aVR is oriented from right arm (+ pole) to left arm and left leg (− pole); Lead aVL is oriented from left arm (+ pole) to right arm and left leg (− pole); and Lead aVF is oriented from left leg (+ pole) to right arm and left arm (− pole). Leads V1, V2 and V3 are oriented on the chest in a posterior to anterior direction. V1 is placed at the fourth intercostal space adjacent to the right sternal border. V2 is placed at the fourth intercostal space adjacent to the left sternal border. V3 is placed halfway between V2 and V4. Leads V4, V5 and V6 are oriented on the chest in a right to left direction. V4 is placed at the fifth intercostal space, midclavicular line. V5 is placed horizontal to V4 on the anterior axillary line. V6 is placed horizontal to V4-5 on the midaxillary line.

An electrocardiogram (ECG/EKG) provides measurement, rhythm, conduction and waveform data.

Measurement data includes heart rate (both atrial and ventricular rates), PR interval (from beginning of P to beginning of QRS complex), QRS duration (width of QRS), QT interval (from beginning of QRS to end of T) and QRS axis in frontal plane.

Rhythm data may include basic rhythm (e.g., normal sinus rhythm or atrial fibrillation) and additional rhythm events (e.g., premature ventricular contractions, premature atrial contractions).

Conduction data includes sino-atrial (SA) atrio-ventricular (AV) and intraventricular (IV) conduction. Conduction abnormalities may be identified, including, but not limited to, second degree SA block; first, second and third degree AV block; IV blocks such as bundle branch, fascicular and non-specific blocks and exit blocks (distal to the sinus or an ectopic pacemaker site).

Waveform data includes P waves, QRS complexes, ST segments, T waves and U waves.

Characteristics of a normal ECG/EKG trace include a heart rate of 50-100 beats per minute; a PR interval of 0.12-0.20 seconds; a QRS duration of 0.06-0.10 seconds; a QT interval of >0.36 seconds and <0.45 seconds for men and >0.36 seconds and <0.46 seconds for women; a frontal plane QRS axis +90° to −30°; normal sinus rhythm; normal sino-atrial (SA), atrio-ventricular (AV) and intraventricular (IV) rhythm; and normal waveform, for example, P wave duration <0.12 seconds; P wave amplitude <2.5 mm; frontal plane P wave axis 0° to +75°; QRS duration <0.10 seconds; and QRS axis range of +90° to −30°.

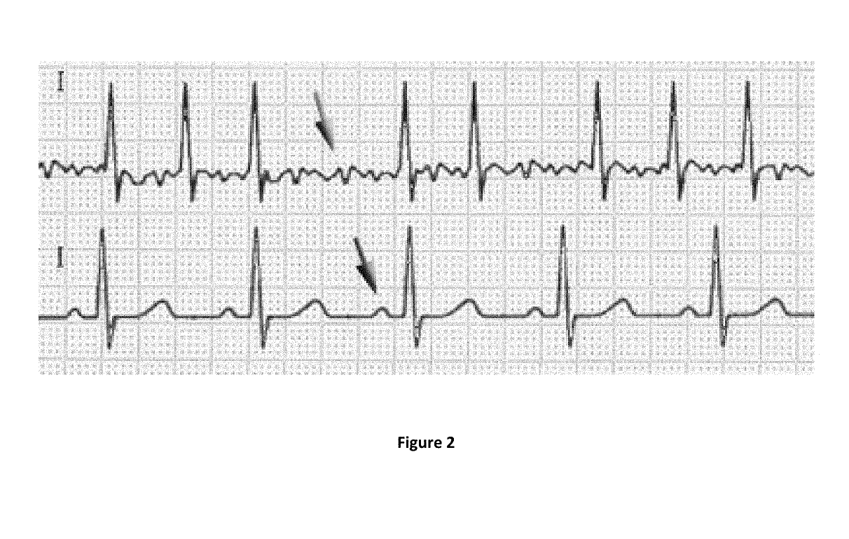

An ECG trace in which there are no P waves and the QRS complexes appear narrow, and at randomly irregular intervals, is the hallmark of atrial fibrillation (FIG. 2).

According to some embodiments, the delivery system of the described invention comprises a particulate composition in a form that is malleable, meaning capable of being extended or shaped or molded, or conformable to a diffuse surface. According to some embodiments, the particulate composition can be provided in form of a filament. According to some embodiments, the particulate composition of the described composition can be provided in form of a cord. According to some embodiments, the particulate composition of the described composition can be provided in form of a thread. According to some embodiments, the particulate composition of the described composition can be provided in form of a string. According to some embodiments, the particulate composition can be provided in form of a film. According to some embodiments, the particulate composition of the described composition can be provided in form of a sheet. According to some embodiments, the form does not disintegrate or fall apart in water.

According to some embodiments, the particulate formulation of the described composition can be provided in form of a spray. According to some embodiments, the spray is a polymeric matrix. Commercially available sprayable polymeric matrices include, without limitation, CoSeal® Surgical Sealant from Baxter Healthcare (Deerfield, Ill.).

According to some embodiments, the particulate formulation of the described composition can be provided in form of a patch. According to some embodiments, the patch is comprised of a bilayer. According to some embodiments, the patch is comprised of a multilayer.

According to some embodiments, the patch is comprised of a controlled-release polymer. According to some embodiments, the polymer is a biodegradable polymer. Biodegradable polymers include, but are not limited to, natural polymers and synthetic polymers. Non-limiting examples of natural polymers include collagen, atelocollagen and gelatin. Non-limiting examples of synthetic polymers include polylactic acid (PLA), polyglycolic acid (PGA), poly(lactic-co-glycolic acid) (PLGA), poly(ethylene glycol) (PEG) 10,000 tetra (4-pentenoate), polycaprolactone, polyparadioxane, polyphosphoesters, polyanhydride, and polyphosphazenes. According to some embodiments, the polymer is a smart polymer. Smart polymers, or environmental responsive polymers, are macromolecules that display physicochemical change in response to environmental stimuli, including, but not limited to, change in temperature, pH, ionic strength, redox potential, biochemical agents, and ultrasound. Advantages of these polymers include, but are not limited to, ease of application, localized delivery of drugs with site-specific action, prolonged delivery period, and decreased systemic drug dosage to minimize the associated side effects. Non-limiting examples of polymers that display thermosensitivity (changes upon temperature change) include poly(N-isopropylacrylamide) (PNIPAAM), poly(ethylene oxide)-poly(propylene oxide)-poly (ethylene oxide) triblock copolymers (PEO-PPO-PEO), poly(ethylene glycol)-poly(lactic acid)-poly(ethylene glycol) triblocks (PEG-PLA-PEG), chitosan grafted with PEG (PEG-g-chitosan) and poly(lactic-co-glycolicacid)-poly(ethylene glycol)-poly(lactic-co-glycolicacid) triblocks (PLGA-PEG-PLGA). Non-limiting examples of pH-sensitive polymeric systems include acrylic acid and methacrylic acid. Polymers that are sensitive to more than one stimulus, for example, pH and temperature, include, but are not limited to PLA/PLGA-PEG diblock, PLA/PLGA-PEG/PLA/PLGA triblock and PLGA-PEG-PLGA triblock.

According to some embodiments, the patch comprises a backing/outer layer (i.e., layer that does not contact a tissue surface or organ surface).

According to some embodiments, the backing/outer layer is comprised of a drug-impermeable material, a liquid-impermeable material or a combination thereof. Backing/outer layer materials include, but are not limited to polyester, polyurethane, polyether, fabrics impregnated with film rendering the fabrics impervious to the drugs and vehicles, regenerated cellulose (e.g., cellophane), acrylonitrile butadiene styrene (ABS) polymer/cellulose acetate, ethyl cellulose, copolymers of plasticized vinylacetate vinylchloride, polyethylene terephthlate, polyethylene, polypropylene, nylon film or nylon fabric impregnated with drug impervious films, polyvinylidene chloride, silicone rubber, natural rubber, polyethylene vinyl acetate, impregnated and coated papers and metallic foils, metalized shaped films of polyvinylchloride (PVC), ABS and other shapeable polymeric sheets or films.

According to some embodiments, the backing/outer layer is comprised of a selectively permeable membrane comprised of a microporous polymer. According to some embodiments, the microporous polymer can be used to exclude transport of compounds having greater than a predetermined molecular weight. Microporous polymers include, without limitation, polycarbonates (i.e., linear polyesters of carbonic acids in which carbonate groups recur in the polymer chain by phosgenation of a dihydroxy aromatic) such as bisphenol A, polyvinylchlorides, polyamides such as polyhexamethylene adipamide and other such polyamides commonly known as “nylon”, modacrylic copolymers such as those formed of polyvinylchloride and acrylonitrile, and styrene-acrylic acid copolymers, polysulfones such as those characterized by diphenylene sulfone groups in a linear chain, halogenated polymers such as polyvinylidene fluoride and polyvinylfluoride, polychloroethers and thermoplastic polyethers, acetal polymers such as polyformaldehyde, acrylic resins such as polyacrylonitrile, polymethyl methacrylate and poly n-butyl methacrylate, polyurethanes, polyimides, polybenzimidazoles, polyvinyl acetate, aromatic and aliphatic polyethers, cellulose esters such as cellulose triacetate, cellulose, collodion, epoxy resins, olefins such as polyethylene and polypropylene, porous rubber, cross-linked poly(ethylene oxide), cross-linked polyvinylpyrrolidone, cross-linked poly(vinyl alcohol); derivatives of polystyrene such as poly (sodium styrenesulfonate) and polyvinylbenzyltrimethyl-ammonium chloride, poly(hydroxyethyl methacrylate), poly(isobutyl vinyl ether), polyisoprenes, polyalkenes, ethylene vinyl acetate copolymers, polyamides, polyurethanes, polyethylene oxides, polyox, polyox blended with polyacrylic acid or Carbopol™, cellulose derivatives such as hydroxypropyl methyl cellulose, hydroxyethyl cellulose, hydroxypropyl cellulose, pectin, starch, guar gum, locust bean gum, and the like.

According to some embodiments, the particulate formulation comprises a plurality of particles. According to some embodiments, the particulate formulation comprises a plurality of milliparticles comprising a therapeutic amount of a first therapeutic agent, wherein the first therapeutic agent is dispersed throughout each milliparticle, adsorbed onto the milliparticles, or is in a core surrounded by a coating. According to some embodiments, the particulate formulation comprises a plurality of microparticles comprising a therapeutic amount of a therapeutic agent, wherein the therapeutic agent is dispersed throughout each microparticle, adsorbed onto the microparticles, or in a core surrounded by a coating. According to some embodiments, the particulate formulation comprises a plurality of nanoparticles comprising a therapeutic amount of a therapeutic agent, wherein the therapeutic agent is dispersed throughout each nanoparticle, adsorbed onto the nanoparticles, or in a core surrounded by a coating. According to some embodiments, the particulate formulation comprises a plurality of picoparticles comprising a therapeutic amount of a therapeutic agent, wherein the therapeutic agent is dispersed throughout each picoparticle, adsorbed onto the picoparticles, or in a core surrounded by a coating. According to some embodiments, the particulate formulation comprises a plurality of femtoparticles comprising a therapeutic amount of a therapeutic agent, wherein the therapeutic agent is dispersed throughout each femtoparticle, adsorbed onto the femtoparticles, or in a core surrounded by a coating.

According to some embodiments, the particles of the particulate formulation are of a distribution of uniform particle size. According to some embodiments, the uniform distribution of particle size is achieved by a homogenization process to form a uniform emulsion comprising, for example, microparticles.

According to some embodiments, the particles comprise a therapeutic agent. According to some embodiments, the therapeutic agent is disposed on or in the particles. According to some embodiments, the therapeutic agent is dispersed throughout the particles. According to some embodiments, a surface of the particles is impregnated with the therapeutic agent. According to some embodiments, the therapeutic agent is adsorbed onto a surface of the particles. According to some embodiments, the therapeutic agent is contained within a core of the particles surrounded by a coating. According to some embodiments, the particles comprise a matrix. According to some embodiments, the matrix comprises the therapeutic agent. According to some embodiments, the matrix is impregnated with the therapeutic agent.

According to some embodiments, the therapeutic agent is an anti-arrhythmic agent. According to some embodiments, the anti-arrhythmic agent is a Class I anti-arrhythmic agent, a Class II anti-arrhythmic agent, a Class III anti-arrhythmic agent, a Class IV anti-arrhythmic agent, a Class V anti-arrhythmic agent, or a combination thereof.

Exemplary Class I anti-arrhythmic agents include, without limitation, quinidine (Quinidex™, Quinaglute™), procainamide (Procan™, Procanbid™) and disopyramide (Norpace™), lidocaine (Xylocaine™) mexilitine (Mexitil™), tocainide (Tonocard™) and phenytoin (Dilantin™), flecainide (Tambocor™), propafenone (Rythmol™) and moricizine.

Exemplary Class II anti-arrhythmic agents include, without limitation, acebutolol, atenolol, bisoprolol, esmolol, metoprolol, timolol, nadolol, propanolol, carvedolol and labetalol.

Exemplary Class III anti-arrhythmic agents include, without limitation, bretylium, ibutilide (Corvert™), dofetilide (Tikosyn™), sotalol (Betapace™) and amiodarone (Cordarone™, Pacerone™).

Class IV anti-arrhythmic agents include, but are not limited to, dihydropyridines, benzothiazepines and phenylalkylamines. Exemplary dihyhdropyridines include amlodipine, isradipine, felodipine, nicardipine, nimodipine and nifedipine. An exemplary benzothiazepine is diltiazem. An exemplary phenylalkylamines includes, without limitation, verapamil.

Exemplary Class V anti-arrhythmic agents include, without limitation, adenosine, digoxin and magnesium.

According to some embodiments, the anti-arrhythmic agent is amiodarone.

According to some embodiments, the anti-arrhythmic agent is a derivative of amiodarone. Derivatives of amiodarone include, but are not limited to, Dronedarone [n-(2-butyl-3-(4-(3-dibutylaminopropoxy)-benzoyl)benzofuran-5-yl)-methanesulfonamide] and KB130015 (KB015) [2-methyl-3-(3,5-diiodo-4-carboxymethoxybenzyl)benzofuran].

According to some embodiments, the anti-arrhythmic agent is a metabolite of amiodarone. Metabolites of amiodarone include, but are not limited, mono-N-desethylamiodarone (B2-O-Et-NH-ethyl), di-N-desethylamiodarone (B2-O-Et-NH₂), and (2-butyl-benzofuran-3-yl)-(4-hydroxy-3,5-diiodophenyl)-methanone (B2) carrying an ethanol side chain [(2-butylbenzofuran-3-yl)-[4-(2-hydroxyethoxy)-3,5-diiodophenyl]-methanone; B2-O-Et-OH].

According to some embodiments, the anti-arrhythmic agent is an analog of amiodarone. Analogs of amiodarone include, but are not limited to, N-dimethylamiodarone (B2-O-Et-N-dimethyl), N-dipropylamiodarone (B2-O-Et-N-dipropyl), B2-O-carrying an acetate side chain [[4-(2-butyl-benzofuran-3-carbonyl)-2,6-diiodophenyl]-acetic acid; B2-O-acetate], B2-O-Et carrying an propionamide side chain (B2-O-Et-propionamide), and B2-0 carrying an ethyl side chain [(2-butylbenzofuran-3-yl)-(4-ethoxy-3,5-diiodophenyl)-methanone; B2-O-Et].

According to some embodiments, the therapeutic agent is an isolated molecule. According to some embodiments, the therapeutic agent is substantially pure.

According to some embodiments, the particles comprise at least one additional therapeutic agent. According to some embodiments, the additional therapeutic agent is disposed on or in the particles. According to some embodiments, the additional therapeutic agent is dispersed throughout the particles. According to some embodiments, a surface of the particles is impregnated with the additional therapeutic agent. According to some embodiments, the additional therapeutic agent is adsorbed onto a surface of the particles. According to some embodiments, the additional therapeutic agent is contained within a core of the particles surrounded by a coating.

According to some embodiments, the additional therapeutic agent is a statin. Non-limiting examples of statins include atorvastatin (Lipitor™) and rosuvastatin (Crestor™). According to some embodiments, the additional therapeutic agent is an omega-3 fatty acid. According to some embodiments, the additional therapeutic agent is ascorbic acid. According to some embodiments, the additional therapeutic agent is N-acetylcysteine (NAC). According to some embodiments, the additional therapeutic agent is sodium nitroprusside (SNP). According to some embodiments, the additional therapeutic agent is an antagonist of angiotensin II. According to some embodiments, the antagonist of angiotensin II is an angiotensin-converting-enzyme (ACE) inhibitor. Exemplary ACE inhibitors include, without limitation, quinapril (Accupril™), perindopril (Aceon™), Ramipril (Altace™), captopril (Capoten™), benazepril (Lotensin™), trandolapril (Mavik™), fosinopril (Monpril™), Lisinopril (Prinivil™, Zestril™), moexipril (Univasc™) and enalapril (Vasotec™). According to some embodiments, the antagonist of angiotensin II is an angiotensin receptor blocker (ARB). Exemplary ARBs include, without limitation, azilsartan (Edarbi™), candesartan (Atacand™), eprosartan (Teveten™), irbesartan (Avapro™), losartan (Cozaar™), olmesartan (Benicar™), telmisartan (Micardis™) and valsartan (Diovan™).

According to some embodiments, the additional therapeutic agent is an anti-inflammatory agent.

According to some embodiments, the anti-inflammatory agent is a steroidal anti-inflammatory agent. The term “steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

According to some embodiments, the anti-inflammatory agent is a nonsteroidal anti-inflammatory agent. The term “non-steroidal anti-inflammatory agent” as used herein refers to a large group of agents that are aspirin-like in their action, including, but not limited to, ibuprofen (Advil™), and naproxen sodium (Aleve™). Additional examples of non-steroidal anti-inflammatory agents include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents.

According to some embodiments, the additional therapeutic agent is a thiazolidinedione (TZD). Non-limiting examples of TZDs include pioglitazone (Actos™) and rosiglitazone (Avandia™).

According to some embodiments, the additional therapeutic agent is an analgesic agent. According to some embodiments, the analgesic agent relieves pain by elevating the pain threshold without disturbing consciousness or altering other sensory modalities. According to some such embodiments, the analgesic agent is a non-opioid analgesic. “Non-opioid analgesics” are natural or synthetic substances that reduce pain but are not opioid analgesics. Examples of non-opioid analgesics include, without limitation, etodolac, indomethacin, sulindac, tolmetin, nabumetone, piroxicam, acetaminophen (Tylenol™), fenoprofen, flurbiprofen, ibuprofen, ketoprofen, naproxen, naproxen sodium, oxaprozin, aspirin, choline magnesium trisalicylate, diflunisal, meclofenamic acid, mefenamic acid, and phenylbutazone. According to some other embodiments, the analgesic is an opioid analgesic. “Opioid analgesics”, “opioid”, or “narcotic analgesics” are natural or synthetic substances that bind to opioid receptors in the central nervous system, producing an agonist action. Examples of opioid analgesics include, without limitation, codeine, fentanyl, hydromorphone, levorphanol, meperidine, methadone, morphine, oxycodone, oxymorphone, propoxyphene, buprenorphine, butorphanol, dezocine, nalbuphine, and pentazocine.

According to some embodiments, the additional therapeutic agent is an anti-infective agent. According to some embodiments, the anti-infective agent is an antibiotic agent. The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, without limitation, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

According to some embodiments, other therapeutic agents, biologically-active agents, drugs, medicaments and inactives may be incorporated into the delivery system for providing a local biological, physiological, or therapeutic effect in the body at various release rates.

According to some embodiments, the particles can be of any order release kinetics, including zero order release, first order release, second order release, delayed release, sustained release, immediate release, and a combination thereof. The particles can include, in addition to therapeutic agent(s), any of those materials routinely used in the art of pharmacy and medicine, including, but not limited to, erodible, nonerodible, biodegradable, or nonbiodegradable material or combinations thereof.

According to some embodiments, the particles are loaded with an average of at least 5% by weight of the therapeutic agent. According to some embodiments, the particles are loaded with an average of at least 10% by weight of the therapeutic agent. According to some embodiments, the particles are loaded with an average of at least 15% by weight of the therapeutic agent. According to some embodiments, the particles are loaded with an average of at least 20% by weight of the therapeutic agent. According to some embodiments, the particles are loaded with an average of at least 25% by weight of the therapeutic agent. According to some embodiments, the particles are loaded with an average of at least 30% by weight of the therapeutic agent. According to some embodiments, the particles are loaded with an average of at least 35% by weight of the therapeutic agent. According to some embodiments, the particles are loaded with an average of at least 40% by weight of the therapeutic agent. According to some embodiments, the particles are loaded with an average of at least 45% by weight of the therapeutic agent. According to some embodiments, the particles are loaded with an average of at least 50% by weight of the therapeutic agent. According to some embodiments, the particles are loaded with an average of at least 55% by weight of the therapeutic agent. According to some embodiments, the particles are loaded with an average of at least 60% by weight of the therapeutic agent. According to some embodiments, the particles are loaded with an average of at least 63% by weight of the therapeutic agent. According to some embodiments, the particles are loaded with an average of at least 65% by weight of the therapeutic agent. According to some embodiments, the particles are loaded with an average of at least 70% by weight of the therapeutic agent. According to some embodiments, the particles are loaded with an average of at least 75% by weight of the therapeutic agent. According to some embodiments, the particles are loaded with an average of at least 80% by weight of the therapeutic agent. According to some embodiments, the particles are loaded with an average of at least 85% by weight of the therapeutic agent. According to some embodiments, the particles are loaded with an average of at least 90% by weight of the therapeutic agent. According to some embodiments, the particles are loaded with an average of at least 95% by weight of the therapeutic agent.

According to some embodiments, the particulate formulation comprises a suspension of particles. According to one embodiment, the particulate formulation comprises a powder suspension of particles. According to some embodiments, the particulate formulation further comprises at least one of a suspending agent, a stabilizing agent and a dispersing agent.

According to some embodiments, the particulate formulation is presented as a solution. According to some embodiments, the particulate formulation is presented as an emulsion.

According to some embodiments, the particulate formulation comprises an aqueous solution of the therapeutic agent in water-soluble form. According to some embodiments, the particulate formulation comprises an oily suspension of the first therapeutic agent. An oily suspension of the first therapeutic agent can be prepared using suitable lipophilic solvents. Exemplary lipophilic solvents or vehicles include, without limitation, fatty oils such as sesame oil, or synthetic fatty acid esters, such as ethyl oleate or triglycerides.

According to some embodiments, the particulate formulation comprises an aqueous suspension of the therapeutic agent. Aqueous injection suspensions, for example, can contain substances which increase the viscosity of the suspension, such as sodium carboxymethyl cellulose, sorbitol, hyaluronic acid, or dextran. Optionally, the suspension can contain suitable stabilizers or agents which increase the solubility of the compounds to allow for the preparation of highly concentrated solutions.

According to some embodiments, the therapeutic agent can be in powder form for constitution with a suitable vehicle, e.g., sterile pyrogen-free water, before use. According to some embodiments, the particulate formulation is dispersed in a vehicle to form a dispersion, with the particles as the dispersed phase, and the vehicle as the dispersion medium.

The particulate formulation can include, for example, microencapsulated dosage forms, and if appropriate, with one or more excipients, encochleated, coated onto microscopic gold particles, contained in liposomes, pellets for implantation into the tissue, or dried onto an object to be rubbed into the tissue. As used herein, the term “microencapsulation” refers to a process in which very tiny droplets or particles are surrounded or coated with a continuous film of biocompatible, biodegradable, polymeric or non-polymeric material to produce solid structures including, but not limited to, nonpareils, pellets, crystals, agglomerates, microspheres, or nanoparticles.

A particulate formulation containing a uniform distribution of microparticle size can be prepared by an emulsion based process. An exemplary microencapsulation process is disclosed and described in U.S. Pat. No. 5,407,609 (entitled Microencapsulation Process and Products Thereof), which is incorporated herein by reference.

According to some embodiments, a process for producing a bioactive agent encapsulated into particles comprises: (a) providing a substantially pure crystalline form of the bioactive agent; (b) adding the substantially pure crystalline form of the bioactive agent to a polymer solution, thereby creating a mixture of the bioactive agent and the polymer solution; (c) homogenizing the mixture to form a disperse phase; (d) mixing the disperse phase with a continuous phase comprising a continuous process medium, thereby forming an emulsion comprising the bioactive agent; (e) forming and extracting the particles comprising the substantially pure bioactive agent; and (f) drying the particles.

It is understood and herein contemplated that where a polymer solution comprises a polymer in an organic solvent forming an oil/water emulsion in the disperse phase, mixing the disperse phase with the continuous phase results in a double emulsion (i.e., a water/oil/water emulsion). Where the polymer solution comprises a polymer in an aqueous solvent such as water, only a single emulsion is formed upon mixing the dispersed phase with the continuous phase.

According to some embodiments, the continuous process medium comprises a surfactant and the bioactive agent saturated with the solvent used in the polymer solution.

The particulate formulation can be in the form of granules, beads, powders, (micro)capsules, emulsions, suspensions, or preparations with protracted release of active compounds, in whose preparation excipients and additives and/or auxiliaries such as disintegrants, binders, coating agents, swelling agents, lubricants, or solubilizers are customarily used as described above. The particulate formulations are suitable for use in a variety of drug delivery systems. See Langer (1990) Science 249, 1527-1533.

Drug Delivery from Bioresorbable Polymers

Site-specific activity generally results if the location in the body into which the formulation is deposited is a fluid-filled space or some type of cavity. This provides high concentrations of the drug at the site where activity is needed, and lower concentrations in the rest of the body, thus decreasing the risk of unwanted systemic side effects.

Site-specific delivery systems, for example, include use of microparticles (of about 1 μm to about 100 μm in diameter), thermoreversible gels (for example, PGA/PEG), and biodegradable polymers (for example, PLA, PLGA) that may be in the form of a film.

The delivery characteristics of the drug and the polymer degradation in vivo also can be modified. For example, polymer conjugation can be used to alter the circulation of the drug in the body and to achieve tissue targeting, reduce irritation and improve drug stability.

Controlled Release Polymeric Drug Delivery Systems

According to some embodiments, the delivery system is a controlled release delivery system. Biodegradable polymeric drug delivery systems that control the release rate of the contained drug in a predetermined manner can overcome practical limitations to targeted delivery. A drug can be attached to soluble macromolecules, such as proteins, polysaccharides, or synthetic polymers via degradable linkages. For example, in animals, antitumor agents such as doxorubicin coupled to N-(2-hydroxypropyl) methacrylamide copolymers showed radically altered pharmacokinetics resulting in reduced toxicity. The half-life of the drug in plasma and the drug levels in the tumor were increased while the concentrations in the periphery decreased. (Kopecek and Duncan, J Controlled Release 6, 315 (1987)). Polymers, such as polyethylene glycol (PEG) can be attached to drugs to either lengthen their lifetime or alter their immunogenicity; drug longevity and immunogenicity also may be affected by biological approaches, including protein engineering and altering glycosylation patterns.

Controlled release systems deliver a drug at a predetermined rate for a definite time period. (Reviewed in Langer, R., “New methods of drug delivery,” Science, 249: 1527-1533 (1990); and Langer, R., “Drug delivery and targeting,” Nature, 392 (Supp.): 5-10 (1998)). Generally, release rates are determined by the design of the system, and are nearly independent of environmental conditions, such as pH. These systems also can deliver drugs for long time periods (days or years). Controlled release systems provide advantages over conventional drug therapies. For example, after ingestion or injection of standard dosage forms, the blood level of the drug rises, peaks and then declines. Since each drug has a therapeutic range above which it is toxic and below which it is ineffective, oscillating drug levels may cause alternating periods of ineffectiveness and toxicity. A controlled release preparation maintains the drug in the desired therapeutic range by a single administration. Other potential advantages of controlled release systems include: (i) localized delivery of the drug to a particular body compartment, thereby lowering the systemic drug level; (ii) preservation of medications that are rapidly destroyed by the body; (iii) reduced need for follow-up care; (iv) increased comfort; and (v) improved compliance. (Langer, R., “New methods of drug delivery,” Science, 249: at 1528).

Optimal control is afforded if the drug is placed in a polymeric material or pump. Polymeric materials generally release drugs by the following mechanisms: (i) diffusion; (ii) chemical reaction, or (iii) solvent activation. The most common release mechanism is diffusion. In this approach, the drug is physically entrapped inside a solid polymer that can then be injected or implanted in the body. The drug then migrates from its initial position in the polymeric system to the polymer's outer surface and then to the body. There are two types of diffusion-controlled systems: reservoirs, in which a drug core is surrounded by a polymer film, which produce near-constant release rates, and matrices, where the drug is uniformly distributed through the polymer system. Drugs also can be released by chemical mechanisms, such as degradation of the polymer, or cleavage of the drug from a polymer backbone. Exposure to a solvent also can activate drug release; for example, the drug may be locked into place by polymer chains, and, upon exposure to environmental fluid, the outer polymer regions begin to swell, allowing the drug to move outward, or water may permeate a drug-polymer system as a result of osmotic pressure, causing pores to form and bringing about drug release. Such solvent-controlled systems have release rates independent of pH. Some polymer systems can be externally activated to release more drug when needed. Release rates from polymer systems can be controlled by the nature of the polymeric material (for example, crystallinity or pore structure for diffusion-controlled systems; the lability of the bonds or the hydrophobicity of the monomers for chemically controlled systems) and the design of the system (for example, thickness and shape). (Langer, R., “New methods of drug delivery,” Science, 249: at 1529).

Polyesters such as lactic acid-glycolic acid copolymers display bulk (homogeneous) erosion, resulting in significant degradation in the matrix interior. To maximize control over release, it is often desirable for a system to degrade only from its surface. For surface-eroding systems, the drug release rate is proportional to the polymer erosion rate, which eliminates the possibility of dose dumping, improving safety; release rates can be controlled by changes in system thickness and total drug content, facilitating device design. Achieving surface erosion requires that the degradation rate on the polymer matrix surface be much faster than the rate of water penetration into the matrix bulk. Theoretically, the polymer should be hydrophobic but should have water-labile linkages connecting monomers. For example, it was proposed that, because of the lability of anhydride linkages, polyanhydrides would be a promising class of polymers. By varying the monomer ratios in polyanhydride copolymers, surface-eroding polymers lasting from 1 week to several years were designed, synthesized and used to deliver nitrosoureas locally to the brain. ((Langer, R., “New methods of drug delivery,” Science, 249: at 1531 citing Rosen et al, Biomaterials 4, 131 (1983); Leong et al, J. Biomed. Mater. Res. 19, 941 (1985); Domb et al, Macromolecules 22, 3200 (1989); Leong et al, J. Biomed. Mater. Res. 20, 51 (1986), Brem et al, Selective Cancer Ther. 5, 55 (1989); Tamargo et al, J. Biomed. Mater. Res. 23, 253 (1989)).

Several different surface-eroding polyorthoester systems have been synthesized. Additives are placed inside the polymer matrix, which causes the surface to degrade at a different rate than the rest of the matrix. Such a degradation pattern can occur because these polymers erode at very different rates, depending on pH, and the additives maintain the matrix bulk at a pH different from that of the surface. By varying the type and amount of additive, release rates can be controlled. ((Langer, R., “New methods of drug delivery,” Science, 249: at 1531 citing Heller, et al, in Biodegradable Polymers as Drug Delivery Systems, M. Chasin and R. Langer, Eds (Dekker, New York, 1990), pp. 121-161)).

According to some embodiments, in order to prolong the effect of a drug, it may be desirable to slow the absorption of the drug. This can be accomplished, for example, by the use of a liquid suspension of crystalline or amorphous material with poor water solubility. The rate of absorption of the drug then depends upon its rate of dissolution which, in turn, may depend upon crystal size and crystalline form. For example, according to some embodiments, a SABER™ Delivery System comprising a high-viscosity base component, is used to provide controlled release of the therapeutic agent. (See U.S. Pat. No. 8,168,217, U.S. Pat. No. 5,747,058 and U.S. Pat. No. 5,968,542, incorporated herein by reference). When the high viscosity SAIB is formulated with drug, biocompatible excipients and other additives, the resulting formulation is liquid enough to inject easily with standard syringes and needles. After injection of a SABER™ formulation, the excipients diffuse away, leaving a viscous depot.

SABER™ formulations comprise a drug and a high viscosity liquid carrier material (HVLCM), meaning nonpolymeric, nonwater soluble liquids with a viscosity of at least 5,000 cP at 37° C. that do not crystallize neat under ambient or physiological conditions. HVLCMs may be carbohydrate-based, and may include one or more cyclic carbohydrates chemically combined with one or more carboxylic acids, such as sucrose acetate isobutyrate (SAIB). HVLCMs also include nonpolymeric esters or mixed esters of one or more carboxylic acids, having a viscosity of at least 5,000 cP at 37° C., that do not crystallize neat under ambient or physiological conditions, wherein when the ester contains an alcohol moiety (e.g., glycerol). The ester may, for example comprise from about 2 to about 20 hydroxy acid moieties.

Additional components can include, without limitation, a rheology modifier, and/or a network former. A rheology modifier is a substance that possesses both a hydrophobic and a hydrophilic moiety used to modify viscosity and flow of a liquid formulation, for example, caprylic/capric triglyceride (Migliol 810), isopropyl myristate (IM or IPM), ethyl oleate, triethyl citrate, dimethyl phthalate, and benzyl benzoate. A network former is a compound that forms a network structure when introduced into a liquid medium. Exemplary network formers include cellulose acetate butyrate, carbohydrate polymers, organic acids of carbohydrate polymers and other polymers, hydrogels, as well as particles such as silicon dioxide, ion exchange resins, and/or fiberglass that are capable of associating, aligning or congealing to form three dimensional networks in an aqueous environment.

According to some embodiments, the particulate composition is effective to release the therapeutic agent(s) over a time period of 12 hours. According to some embodiments, the particulate composition is effective to release the therapeutic agent(s) over a time period of from 12 hours to 4 days, i.e., at least 12 hours, at least 24 hours, at least 36 hours, at least 48 hours, at least 60 hours, at least 72 hours, at least 84 hours, or no more than 4 days, no more than 3 days, no more than 2 days, no more than 1 day. According to some embodiments, the particulate composition is effective to release the therapeutic agent(s) gradually over a time period of 3-5 days (sustained release), i.e., at least 3 days, at least 4 days, or no more than 5 days

According to some embodiments, the controlled release system is capable of releasing one-half of the therapeutic agent from the delivery system at the site of delivery within 6 hours to 5 days in vivo. According to one embodiment, the controlled release system is capable of releasing one-half of the therapeutic agent from the delivery system at the site of delivery within 6 hours. According to one embodiment, the controlled release system is capable of releasing one-half of the therapeutic agent from the delivery system at the site of delivery within 7 hours. According to one embodiment, the controlled release system is capable of releasing one-half of the therapeutic agent from the delivery system at the site of delivery within 8 hours. According to one embodiment, the controlled release system is capable of releasing one-half of the therapeutic agent from the delivery system at the site of delivery within 9 hours. According to one embodiment, the controlled release system is capable of releasing one-half of the therapeutic agent from the delivery system at the site of delivery within 10 hours. According to one embodiment, the controlled release system is capable of releasing one-half of the therapeutic agent from the delivery system at the site of delivery within 11 hours. According to one embodiment, the controlled release system is capable of releasing one-half of the therapeutic agent from the delivery system at the site of delivery within 12 hours. According to one embodiment, the controlled release system is capable of releasing one-half of the therapeutic agent from the delivery system at the site of delivery within 13 hours. According to one embodiment, the controlled release system is capable of releasing one-half of the therapeutic agent from the delivery system at the site of delivery within 14 hours. According to one embodiment, the controlled release system is capable of releasing one-half of the therapeutic agent from the delivery system at the site of delivery within 15 hours. According to one embodiment, the controlled release system is capable of releasing one-half of the therapeutic agent from the delivery system at the site of delivery within 16 hours. According to one embodiment, the controlled release system is capable of releasing one-half of the therapeutic agent from the delivery system at the site of delivery within 17 hours. According to one embodiment, the controlled release system is capable of releasing one-half of the therapeutic agent from the delivery system at the site of delivery within 18 hours. According to one embodiment, the controlled release system is capable of releasing one-half of the therapeutic agent from the delivery system at the site of delivery within 24 hours. According to one embodiment, the controlled release system is capable of releasing one-half of the therapeutic agent from the delivery system at the site of delivery within 36 hours. According to one embodiment, the controlled release system is capable of releasing one-half of the therapeutic agent from the delivery system at the site of delivery within 48 hours. According to one embodiment, the controlled release system is capable of releasing one-half of the therapeutic agent from the delivery system at the site of delivery within 72 hours. According to one embodiment, the controlled release system is capable of releasing one-half of the therapeutic agent from the delivery system at the site of delivery within 90 hours.

According to some embodiments, because the described delivery system is malleable, it can be delivered to an implant site, where it is effective to ensure drug distribution and uniform drug delivery throughout the implant site. According to some embodiments, the delivery system can be manipulated so as to contact a surface of tissue susceptible to arrhythmia. According to some embodiments, the surface of tissue susceptible to arrhythmia comprises an atrium. According to some embodiments, the atrium is a right atrium, a left atrium, or a combination thereof. According to some embodiments, the surface of tissue susceptible to arrhythmia comprises a superior pulmonary vein, a superior vena cava, a coronary sinus or combination thereof. According to some embodiments, the arrhythmia is atrial fibrillation. According to some embodiments, the described delivery system can adhere to the surface susceptible to arrhythmia. According to some embodiments, the described delivery system can conform to contours of the surface. According to some embodiments, the described delivery system can conform to walls, spaces, or voids in the body. According to some embodiments, the described delivery system can fill the spaces or voids.

According to some embodiments, the total dose of the therapeutic agent administered locally is much lower than that administered systemically so the risk of other and unknown side effects is lower. Because the concentration of the therapeutic agent locally where it exerts its effect is higher and the plasma concentration is lower than when the therapeutic agent is administered systemically, this results in a localized pharmacological effect at the site of implantation, with less effect in the body. Accordingly, unwanted side effects in the body are less likely to occur.

According to some embodiments, the release of the therapeutic agent at the site of delivery is effective to produce a predominantly localized pharmacologic effect over a desired amount of time. According to some embodiments, the release of the therapeutic agent at the site of delivery is effective to produce a predominantly localized pharmacologic effect for 1 day. According to some embodiments, the release of the therapeutic agent at the site of delivery is effective to produce a predominantly localized pharmacologic effect for 2 days. According to some embodiments, the release of the therapeutic agent at the site of delivery is effective to produce a predominantly localized pharmacologic effect for 3 days. According to some embodiments, the release of the therapeutic agent at the site of delivery is effective to produce a predominantly localized pharmacologic effect for 4 days. According to some embodiments, the release of the therapeutic agent at the site of delivery is effective to produce a predominantly localized pharmacologic effect for 5 days. According to some embodiments, the release of the therapeutic agent at the site of delivery is effective to produce a predominantly localized pharmacologic effect for 6 days. According to some embodiments, the release of the therapeutic agent at the site of delivery is effective to produce a predominantly localized pharmacologic effect for 7 days.

According to some embodiments, the release of the therapeutic agent at the site of delivery is effective to produce a diffuse pharmacologic effect throughout an implant site over a desired amount of time, where the desired amount of time is the time necessary to prevent or reduce the incidence or severity of atrial fibrillation. According to some embodiments, the release of the therapeutic agent is effective to produce a diffuse pharmacologic effect at the implant site for at least 1 day. According to some embodiments, the release of the therapeutic agent is effective to produce a diffuse pharmacologic effect at the implant site for at least 2 days. According to some embodiments, the release of the therapeutic agent is effective to produce a diffuse pharmacologic effect at the implant site for at least 3 days. According to some embodiments, the release of the therapeutic agent is effective to produce a diffuse pharmacologic effect at the implant site for at least 4 days. According to some embodiments, the release of the therapeutic agent is effective to produce a diffuse pharmacologic effect at the implant site for at least 5 days. According to some embodiments, the release of the therapeutic agent is effective to produce a diffuse pharmacologic effect at the implant site for at least 6 days. According to some embodiments, the release of the therapeutic agent is effective to produce a diffuse pharmacologic effect at the implant site for at least 7 days.

Particle Polymer Matrix

According to some embodiments, the particulate composition comprises a polymer matrix. According to some embodiments, the polymer matrix is a naturally occurring biopolymer matrix, a synthetic polymer matrix, or a combination thereof. According to some embodiments, the polymer is a slow release compound. According to some embodiments, the polymer is a biodegradable polymer. According to some embodiments, the polymer is selected from the group consisting of a polyester, a polyethylene glycol polymer, a polyamino-derived biopolymer, a polyanhydride, a polyorthoester, a polyphosphazene, a sucrose acetate isobutyrate (SAIB), a photopolymerizable biopolymer, a polyglycolic acid (PGA), a polylactic acid (PLA); a polycaprolactone; or a thermoreversible gel. According to some embodiments, the polymer is a copolymer containing polyglycolic acid formed with trimethylene carbonate, polylactic acid (PLA), or polycaprolactone; a poly (lactic-co-glycolide)(PLGA); a poly D, L (lactic co-caprolactone) copolymer; or a copolymer comprising a polyester and polyethylene glycol.

Both non-biodegradable and biodegradable polymeric materials can be used in the manufacture of particles for delivering the therapeutic agents. Such polymers can be natural or synthetic polymers. The polymer is selected based on the period of time over which release is desired. Exemplary bioadhesive polymers include, without limitation, bioerodible hydrogels as described by Sawhney et al in Macromolecules 26: 581-587 (1993), the teachings of which are incorporated herein. According to some embodiments, the bioadhesive polymer is hyaluronic acid. Exemplary bioerodible hydrogels include, but are not limited to, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

According to some embodiments, the polymer enhances aqueous solubility of the particulate formulation. Exemplary polymers include, without limitation, polyethylene glycol, poly-(d-glutamic acid), poly-(1-glutamic acid), poly-(d-aspartic acid), poly-(1-aspartic acid) and copolymers thereof. According to some embodiments, the polyglutamic acids have a molecular weight between about 5,000 to about 100,000, a molecular weight between about 20,000 and about 80,000, or a molecular weight between about 30,000 and about 60,000. According to some embodiments, the polymer is conjugated via an ester linkage to one or more hydroxyls of an inventive epothilone using a protocol as described by U.S. Pat. No. 5,977,163 which is incorporated herein by reference. Exemplary conjugation sites include the hydroxyl off carbon-21 in the case of 21-hydroxy-derivatives. Other conjugation sites include, but are not limited, to the hydroxyl off carbon 3 and/or the hydroxyl off carbon 7.

According to some embodiments, the matrix comprises a polyglycolide (PGA) matrix. PGA is a linear aliphatic polyester developed for use in sutures. Studies have reported PGA copolymers formed with trimethylene carbonate, polylactic acid (PLA), and polycaprolactone. Some of these copolymers may be formulated as microparticles for sustained drug release.

According to some embodiments, the matrix comprises a polyester-polyethylene glycol matrix. Polyester-polyethylene glycol compounds are soft and may be used for drug delivery.

According to some embodiments, the matrix comprises a poly (amino)-derived biopolymer. Exemplary poly (amino)-derived biopolymers include, but are not limited to, those containing lactic acid and lysine as the aliphatic diamine (see, for example, U.S. Pat. No. 5,399,665), and tyrosine-derived polycarbonates and polyacrylates. Modifications of polycarbonates may alter the length of the alkyl chain of the ester (ethyl to octyl), while modifications of polyarylates may further include altering the length of the alkyl chain of the diacid (for example, succinic to sebasic), which allows for a large permutation of polymers and great flexibility in polymer properties.

According to some embodiments, the matrix comprises a polyanhydride matrix. Polyanhydrides are prepared by the dehydration of two diacid molecules by melt polymerization (see, for example, U.S. Pat. No. 4,757,128). These polymers degrade by surface erosion (as compared to polyesters that degrade by bulk erosion). The release of the therapeutic agent can be controlled by the hydrophilicity of the monomers chosen.

According to some embodiments, the matrix comprises a photopolymerizable biopolymer. Exemplary photopolymerizable biopolymers include, without limitation, lactic acid/polyethylene glycol/acrylate copolymers.

According to some embodiments, the matrix comprises a hydrogel. Hydrogels generally comprise a variety of polymers, including hydrophilic polymers, acrylic acid, acrylamide and 2-hydroxyethylmethacrylate (HEMA).

According to some embodiments, the matrix comprises a naturally-occurring biopolymer. Exemplary naturally-occurring biopolymers include, but are not limited to, protein polymers, collagen, polysaccharides, and photopolymerizable compounds.

According to some embodiments, the matrix comprises a protein polymer. Exemplary protein polymers synthesized from self-assembling protein polymers include, for example, silk fibroin, elastin, collagen, and combinations thereof.

According to some embodiments, the matrix comprises a naturally-occurring polysaccharide. Exemplary naturally-occurring polysaccharides include, but are not limited to, chitin and its derivatives, hyaluronic acid, dextran and cellulosic (which generally are not biodegradable without modification), and sucrose acetate isobutyrate (SAIB). Hyaluronic acid (HA), which is composed of alternating glucuronidic and glucosaminidic bonds and is found in mammalian vitreous humor, extracellular matrix of the brain, synovial fluid, umbilical cords and rooster combs from which it is isolated and purified, also can be produced by fermentation processes.

According to some embodiments, the matrix comprises a chitin matrix. Chitin is composed predominantly of 2-acetamido-2-deoxy-D-glucose groups and is found in yeast, fungi and marine invertebrates (shrimp, crustaceous) where it is a principal component of the exoskeleton. Chitin is not water soluble and the deacetylated chitin, chitosan, only is soluble in acidic solutions (such as, for example, acetic acid). Studies have reported chitin derivatives that are water soluble, very high molecular weight (greater than 2 million Daltons), viscoelastic, non-toxic, biocompatible and capable of crosslinking with peroxides, gluteraldehyde, glyoxal and other aldehydes and carbodiamides, to form gels.

Pharmaceutical Carrier

According to some embodiments, the pharmaceutical composition comprises a pharmaceutically acceptable carrier.

According to some embodiments, the pharmaceutically acceptable carrier is a solid carrier or excipient. According to some embodiments, the pharmaceutically acceptable carrier is a gel-phase carrier or excipient. Examples of carriers or excipients include, but are not limited to, calcium carbonate, calcium phosphate, various monomeric and polymeric sugars (including without limitation hyaluronic acid), starches, cellulose derivatives, gelatin, and polymers. An exemplary carrier can also include saline vehicle, e.g. hydroxyl propyl methyl cellulose (HPMC) in phosphate buffered saline (PBS). According to some embodiments, the pharmaceutically acceptable carrier is a buffer solution. Exemplary buffer solutions can include without limitation a phosphate buffered saline (PBS) solution.

According to some embodiments, the pharmaceutically acceptable carrier imparts stickiness to the composition. According to some embodiments, the pharmaceutically acceptable carrier comprises hyaluronic acid.

According to some embodiments, the pharmaceutically acceptable carrier includes, but is not limited to, a gel, slow-release solid or semisolid compound, optionally as a sustained release gel. According to some such embodiments, the therapeutic agent is embedded into the pharmaceutically acceptable carrier. According to some embodiments, the therapeutic agent is coated on the pharmaceutically acceptable carrier. The coating can be of any desired material, preferably a polymer or mixture of different polymers. Optionally, the polymer can be utilized during the granulation stage to form a matrix with the active ingredient so as to obtain a desired release pattern of the active ingredient. The gel, slow-release solid or semisolid compound is capable of releasing the active agent over a desired period of time. The gel, slow-release solid or semisolid compound can be implanted in a tissue for example, but not limited to, in close proximity to a surface susceptible to atrial fibrillation.

According to some embodiments, the pharmaceutically acceptable carrier comprises a slow-release solid compound. According to one such embodiment, the therapeutic agent is embedded in the slow-release solid compound or coated on the slow-release solid compound. According to yet some embodiments, the pharmaceutically acceptable carrier comprises a slow-release particle containing the therapeutic agent.

Exemplary buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

According to some embodiments, the pharmaceutically acceptable carrier is a gel compound, such as a biodegradable hydrogel, e.g., glyceryl monooleate (GMO). Many hydrogels, polymers, hydrocarbon compositions and fatty acid derivatives having similar physical/chemical properties with respect to viscosity/rigidity may function as a semisolid delivery system. According to some embodiments, the hydrogel incorporates and retains significant amounts of water, which eventually will reach an equilibrium content in the presence of an aqueous environment.

According to one embodiment, a GMO hydrogel delivery system can be produced by heating GMO above its melting point (40-50° C.) and by adding a warm aqueous-based buffer or electrolyte solution, such as, for example, phosphate buffer or normal saline, which thus produces a three-dimensional structure. The aqueous-based buffer may be comprised of other aqueous solutions or combinations containing semi-polar solvents.

GMO provides a predominantly lipid-based hydrogel, which has the ability to incorporate lipophilic materials. GMO further provides internal aqueous channels that incorporate and deliver hydrophilic compounds. It is recognized that at room temperature (25° C.), the gel system may exhibit differing phases which comprise a broad range of viscosity measures.

According to some embodiments, two gel system phases are utilized due to their properties at room temperature and physiologic temperature (about 37° C.) and pH (about 7.4). Within the two gel system phases, the first phase is a lamellar phase of approximately 5% to approximately 15% H₂O content and approximately 95% to approximately 85% GMO content. The lamellar phase is a moderately viscous fluid that may be easily manipulated, poured and injected. The second phase is a cubic phase consisting of approximately 15% to approximately 40% H₂O content and approximately 85%-60% GMO content. It has an equilibrium water content of approximately 35% to approximately 40% by weight. The term “equilibrium water content” as used herein refers to maximum water content in the presence of excess water. Thus the cubic phase incorporates water at approximately 35% to approximately 40% by weight. The cubic phase is highly viscous. The viscosity exceeds 1.2 million centipoise (cp) when measured by a Brookfield viscometer; where 1.2 million cp is the maximum measure of viscosity obtainable via the cup and bob configuration of the Brookfield viscometer.

Alternatively, according to some embodiments, modified formulations and methods of production are utilized such that the nature of the delivery system is altered, or in the alternative, aqueous channels contained within the delivery system are altered. Thus, various therapeutic agents in varying concentrations may diffuse from the delivery system at differing rates, or be released therefrom over time via the aqueous channels of the delivery system. Hydrophilic substances may be utilized to alter the consistency or therapeutic agent release by alteration of viscosity, fluidity, surface tension or the polarity of the aqueous component. For example, glyceryl monostearate (GMS), which is structurally identical to GMO with the exception of a double bond at Carbon 9 and Carbon 10 of the fatty acid moiety rather than a single bond, does not gel upon heating and the addition of an aqueous component, as does GMO. However, because GMS is a surfactant, GMS is miscible in water up to approximately 20% weight/weight. The term “surfactant” as used herein refers to a surface active agent that is miscible in water in limited concentrations as well as polar substances. Upon heating and stirring, the 80% H₂O/20% GMS combination produces a spreadable paste having a consistency resembling hand lotion. The paste then is combined with melted GMO so as to form the cubic phase gel possessing a high viscosity referred to above.

According to some embodiments, a hydrolyzed gelatin, such as commercially available Gelfoam™, can be utilized for altering the aqueous component. Approximately 6.25% to 12.50% concentration of Gelfoam™ by weight may be placed in approximately 93.75% to 87.50% respectively by weight H₂O or another aqueous based buffer. Upon heating and stirring, the H₂O (or other aqueous buffer)/Gelfoam™ combination produces a thick gelatinous substance. The resulting substance is combined with GMO; a product so formed swells and forms a highly viscous, translucent gel being less malleable in comparison to neat GMO gel alone.

According to some embodiments, polyethylene glycols (PEG's) can be utilized for altering the aqueous component to aid in drug solubilization. Approximately 0.5% to 40% concentration of PEG's (depending on PEG molecular weight) by weight can be placed in approximately 99.5% to 60% H₂O or other aqueous based buffer by weight. Upon heating and stirring, the H₂O (or other aqueous buffer)/PEG combination produces a viscous liquid to a semisolid substance. The resulting substance is combined with GMO, whereby a product so formed swells and forms a highly viscous gel.

According to some embodiments, the therapeutic agent releases from the delivery system through diffusion, conceivably in a biphasic manner. A first phase may involve, for example, a lipophilic drug contained within the lipophilic membrane that diffuses therefrom into the aqueous channel. The second phase may involve diffusion of the drug from the aqueous channel into the external environment. Being lipophilic, the drug may orient itself inside the GMO gel within its proposed lipid bi-layer structure. Thus, incorporating greater than approximately 7.5% of the drug by weight into GMO causes a loss of the integrity of the three-dimensional structure whereby the gel system no longer maintains the semisolid cubic phase, and reverts to the viscous lamellar phase liquid. According to some embodiments, about 1% to about 45% of therapeutic agent is incorporated by weight into a GMO gel at physiologic temperature without disruption of the normal three-dimensional structure. As a result, this system can allow for increased flexibility with drug dosages.

Alternatively, the described invention may provide a delivery system, which acts as a vehicle for local delivery of therapeutic agents, comprising a lipophilic, hydrophilic or amphophilic, solid or semisolid substance, heated above its melting point and thereafter followed by inclusion of a warm aqueous component so as to produce a gelatinous composition of variable viscosity based on water content. Therapeutic agent(s) is/are incorporated and dispersed into the melted lipophilic component or the aqueous buffer component prior to mixing and formation of the semisolid system. The gelatinous composition is placed within the semisolid delivery apparatus for subsequent placement, or deposition. Being malleable, the gel system is easily delivered and manipulated via the semisolid delivery apparatus in an implant site, where it can adhere, conform, or a combination thereof, to contours of the implantation site, spaces, or other voids in the body as well as completely filling all voids existing.

According to some embodiments, the pharmaceutical composition further comprises a preservative agent. According to some such embodiments, the pharmaceutical composition is presented in a unit dosage form. Exemplary unit dosage forms include, but are not limited to, ampoules or multi-dose containers.

According to some embodiments, the pharmaceutical composition may further comprise an adjuvant. Exemplary adjuvants include, but are not limited to, preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. Isotonic agents, for example, sugars, sodium chloride and the like, can also be included. Prolonged absorption of an injectable pharmaceutical form can be brought about by the use of agents delaying absorption, for example, aluminum monostearate and gelatin.

Formulations of the delivery system can be sterilized, for example, by terminal gamma irradiation, filtration through a bacterial-retaining filter or by incorporating sterilizing agents in the form of sterile solid compositions that may be dissolved or dispersed in sterile water or other sterile injectable medium just prior to use.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The described invention may be embodied in other specific forms without departing from the spirit or essential attributes thereof and, accordingly, reference should be made to the appended claims, rather than to the foregoing specification, as indicating the scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Animal Model for Atrial Fibrillation (AF)

Domestic goats weighing between 49 and 75 kg are randomized into 2 groups: a control group (particulate delivery system of the described invention with no amiodarone) and a treatment group (particulate delivery system of the described invention comprising a pharmaceutical composition comprising a particulate formulation containing a therapeutic amount of amiodarone (Sigma-Aldrich, St. Louis, Mo.)).

Anesthesia is induced by thiopental 20 mg/kg IV, and maintained with 2-3% isoflurane in a 1:1 mixture of oxygen and air. Buprenorphine (10 μg/kg IV) is used for analgesia. Throughout the procedure, limb-lead ECG, arterial blood pressure, endexpiratory CO₂ and oxygen saturation (pulse oximeter) are monitored. A rectal temperature of 38 to 39° C. is maintained by an external heating pad. Fluid loss is compensated with saline (0.9%) at 5 to 8 mL/kg/h via a peripheral venous catheter. After a right intercostal thoracotomy, the pericardium above the right atrial lateral wall is opened, in the animals planned for electrophysiological studies, 3 silicone patches of 10×10 mm, each containing 4 silver unipolar electrodes (1.5 mm diameter, 5 mm interelectrode distance), are sutured in line to the right atrial lateral wall, between the cranial and caudal caval veins. Baseline measurements are taken, and the particulate delivery system of the described invention with no amiodarone is applied to the right atrial lateral wall of control group animals; and the particulate delivery system of the described invention comprising a pharmaceutical composition comprising a particulate formulation containing a therapeutic amount of amiodarone is applied to the right atrial lateral wall of treatment group animals; and measurements are repeated after 1 h. The pericardium is approximated and sutured, and the thorax is closed, while the electrode leads are tunneled subcutaneously to the neck and exteriorized. Animals receive twice-daily buprenorphine (10 μg/kg IM) postoperatively for 3 days. Prophylactic ampicillin (20 mg/kg IM) is given before and after surgery.

Measurements are performed during the surgical procedure and 1, 2, 3, 4, 7, 9, 11, 14, 16, 18, 21, 23, 25 and 28 days after the surgical procedure. An electrophysiology monitoring system with an integrated amplifier/stimulator (EPTracer 38, CardioTek, Maastricht-Airport, The Netherlands) is used to simultaneously record limb-lead ECG and six bipolar electrograms from the 3 silicone patches sutured to the right atrial lateral wall. ECG parameters (RR, PQ, QJ, and maximal Tₚₑₐₖ-T_{end} intervals, and P and QRS widths) are measured during normal sinus rhythm. Atrial effective refractory periods (ERP) are measured during bipolar pacing at 4 times the threshold. After 10 basic stimuli (SI) with a 400-ms interval, an extra-stimulus (S2) is applied. Starting from within the refractory period, the S1-S2 interval is increased in steps of 2 ms. The longest S1-S2 interval that failed to capture is defined as the ERP. Atrial conduction times are recorded during 400-ms interval pacing at the silicone patch closest to the cranial caval vein. The time between stimulus artifact and the corresponding atrial deflection of the electrogram at the silicone patch closest to the caudal caval vein (i.e. the conduction delay across the crista terminalis) is defined as the conduction time. Vulnerability to atrial tachyarrhythmias is evaluated by applying 50-Hz burst pacing during 1 s. A rapid atrial response (RAR) is considered inducible if a rapid irregular rhythm lasting >1 s occurs after the burst stimulus. For quantification of RAR inducibility, the percentages of RAR inductions out of 20 attempts are calculated. Duration of RAR episodes is measured and the medians are calculated.

At days 1, 2, 3, 4, 7, 9, 11, 14, 16, 18, 21, 23, 25 and 28 after application of the delivery system of the described invention, blood samples are drawn from a peripheral vein of the hind limb via a venflon cannula and collected in EDTA-containing vacutainer tubes (Becton-Dickinson, Franklin Lakes, N.J.). After centrifugation, the plasma is stored at −80° C. Goats from the treatment group are euthanized with intravenous sodium pentobarbitone at 3 h, and 1, 7, 14, 21 and 28 days after application of the delivery system of the described invention. Immediately after euthanasia, the heart is excised and the lateral right atrium and free-wall sections of 4 cm² from the left atrium and both ventricles are dissected. Samples are also taken from the pericardium overlying the right atrium, pericardial fluid, the medial part of the cranial lobe of the right lung, the skeletal muscle of the hind limb, liver and abdominal fat. Tissue samples are frozen in liquid nitrogen and stored at −80° C. Myocardial samples are freeze-microtomed parallel to the epicardial surface in slices of 500 μm for measurement of transmural drug concentration gradients. Amiodarone concentrations in plasma, pericardial fluid and tissue samples are determined by high-performance liquid chromatography (HPLC) coupled with ultraviolet (UV) detection.

Data are presented as mean±standard deviation (SD). A Kruskal-Wallis test is used for multiple comparisons, a Mann-Whitney U test is used for calculating between group effects, a Wilcoxon signed rank test is used for calculating within-group effects, and a Bonferroni correction is used. Differences are defined statistically significant at a two-tailed P value of <0.05.

### Example 2. Postoperative Atrial Fibrillation (AF)

Patients undergoing cardiac surgery (e.g., coronary artery bypass graft surgery (CABG)) are randomized into 2 groups: a control group (particulate delivery system of the described invention with no amiodarone) and a treatment group (particulate delivery system of the described invention comprising a pharmaceutical composition comprising a particulate formulation containing a therapeutic amount of amiodarone (Sigma-Aldrich, St. Louis, Mo.)). Prior to closure of the sternum, the particulate delivery system of the described invention with no amiodarone is applied to the right atrial lateral wall, left atrial appendage and transverse sinus area of control group patients; and the particulate delivery system of the described invention comprising a pharmaceutical composition comprising a particulate formulation containing a therapeutic amount of amiodarone is applied to the right atrial lateral wall, left atrial appendage and transverse sinus area of treatment group patients. The pericardium is approximated with interrupted suture and a single mediasternal chest tube is placed retromediastinal above the pericardium. Myocardial venous blood sampling is obtained from the coronary venous sinus under fluoroscopic control during postoperative day 3 and right atrial endomyocardial biopsy is performed from areas adjacent to the fossa ovalis for measurements of myocardial amiodarone concentration and abdominal extraperitoneal adipose tissue within the chest tube site is biopsied during chest tube removal on postoperative day 3 respectively. Amiodarone plasma concentrations are measured in blood drawn from a peripheral vein during postoperative day 2 and day 5 using high-performance liquid chromatography (HP-Series 1090, Hewlett Packard, Palo Alto, Calif.). A cardiac enzyme panel is measured on day 1, 3, 5 and before discharge to monitor local atrial injury. ECG/EKG parameters (RR, PQ, QT, maximal Tₚₑₐₖ-T_{end} intervals, P and QRS widths) are measured daily until postoperative day 14.

All values are expressed as the mean±standard deviation (SD). Statistical analysis comparing data between the two groups is performed with X² test for categorical variables. Continuous variables are compared by means of two-tailed Student's t-tests and Kruskal-Wallis test. Data collected are analyzed using a statistical system software such as the number cruncher software (NCSS, Kaysville, Utah). Differences are defined statistically significant at a two-tailed P value of <0.05.

### EQUIVALENTS

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for reducing incidence or severity of atrial fibrillation in a subject at risk thereof, the method comprising
(a) providing a delivery system in a form that is malleable comprising a pharmaceutical composition containing a particulate formulation containing a plurality of particles comprising a therapeutic amount of an anti-arrhythmic agent and a pharmaceutically acceptable carrier;
(b) administering the malleable delivery system at an implant site in contact with a surface susceptible to atrial fibrillation, wherein
(i) the malleable delivery system is effective to contact a surface of a tissue susceptible to atrial fibrillation, adhere to the surface susceptible to atrial fibrillation; conform to contours of the surface susceptible to atrial fibrillation; or a combination thereof; and
(ii) release of the therapeutic agent at the implant site may be effective to produce a predominantly localized pharmacologic effect over a desired amount of time, where the desired amount of time is the time necessary to reduce the incidence or severity of atrial fibrillation.

2. The method according to claim 1, wherein the anti-arrhythmic agent is selected from the group consisting of a Class I anti-arrhythmic agent, a Class II antiarrhythmic agent, a Class III anti-arrhythmic agent, a Class IV anti-arrhythmic agent, a Class V anti-arrhythmic agent and a combination thereof.

3. The method according to claim 2, wherein the anti-arrhythmic agent is a Class III anti-arrhythmic agent.

4. The method according to claim 3, wherein the Class III anti-arrhythmic agent is amiodarone, a derivative of amiodarone, a metabolite of amiodarone, an analog of amiodarone or a combination thereof.

5. The method according to claim 4, wherein the derivative of amiodarone is dronedarone [n-(2-butyl-3-(4-(3-dibutylaminopropoxy)-benzoyl)benzofuran-5-yl)-methanesulfonamide] or KB130015 (KB015) [2-methyl-3-(3,5-diiodo-4-carboxymethoxybenzyl)benzofuran].

6. The method according to claim 4, wherein the metabolite of amiodarone is mono-N-desethylamiodarone (B2-O-Et-NH-ethyl), di-N-desethylamiodarone (B2-O-Et-NH₂) or (2-butyl-benzofuran-3-yl)-(4-hydroxy-3,5-diiodophenyl)-methanone (B2) carrying an ethanol side chain [(2-butylbenzofuran-3-yl)-[4-(2-hydroxyethoxy)-3,5-diiodophenyl]-methanone (B2-O-Et-OH)].

7. The method according to claim 4, wherein the analog of amiodarone is N-dimethylamiodarone (B2-O-Et-N-dimethyl), N-dipropylamiodarone (B2-O-Et-N-dipropyl), B2-O-carrying an acetate side chain [[4-(2-butyl-benzofuran-3-carbonyl)-2,6-diiodophenyl]-acetic acid; B2-O-acetate], B2-O-Et carrying an propionamide side chain (B2-O-Et-propionamide), or B2-O carrying an ethyl side chain [(2-butylbenzofuran-3-yl)-(4-ethoxy-3,5-diiodophenyl)-methanone; B2-O-Et].

8. The method according to claim 1, wherein the surface susceptible to atrial fibrillation is an atrium, a superior pulmonary vein, a superior vena cava vein, or a coronary sinus.

9. The method according to claim 1, further comprising releasing one-half of the therapeutic agent from the delivery system at the implant site within 6 hours; within 12 hours, within 1 day, within 2 days, within 3 days, within 4 days, or within 5 days in vivo.

10. The method according to claim 1, wherein the particulate formulation is in form of a filament, a cord, a thread, a string, a film, a sheet or a patch.

11. The method according to claim 1, wherein the therapeutic agent is dispersed throughout the particle, adsorbed onto the particle, or contained in a core surrounded by a coating.

12. The method according to claim 1, wherein a surface of the particle is impregnated with the therapeutic agent.

13. The method according to claim 1, wherein the particles comprise a matrix.

14. The method according to claim 13, wherein the matrix is impregnated with the therapeutic agent.

15. The method according to claim 1, wherein the delivery system is impregnated with the therapeutic agent.

16. The method according to claim 13, wherein the therapeutic agent is entrapped by the matrix.

17. The method according to claim 1, wherein the therapeutic agent is released from the malleable delivery system into the implant site.

18. The method according to claim 1, wherein the subject at risk of atrial fibrillation is characterized by etiological factors or genetic factors.

19. The method according to claim 18, wherein the etiological factors are selected from the group consisting of age, structural remodeling, congestive heart failure (CHF), hypertension, valvular heart disease, coronary artery disease (CAD), peri- or myocarditis, atrial myxomas, hypertrophic cardiomyopathy, alcohol consumption, hyperthyroidism, sleep apnea, obesity and a combination thereof.

20. The method according to claim 18, wherein the genetic factors are selected from the group consisting of a gene encoding myocardial potassium (K⁺) channels, a gene encoding sodium (Na⁺) channels, a gene encoding potassium (K⁺)-adenosine triphosphate channels, nucleoporin-155 (NUP155), gap junction protein connexion 40 (GJAS), atrial natriuretic peptide (NPPA), a single-nucleotide polymorphism (SNP) on chromosome 4q25 and a combination thereof.

21. The method according to claim 20, wherein the gene encoding myocardial potassium (K⁺) channels are selected from the group consisting of KCNQ1, KCNA5, KCNE5, KCNJ2, KCNE2 and a combination thereof.

22. The method according to claim 20, wherein the gene encoding sodium (Na⁺) channels are selected from the group consisting of SCN5A, SCN1B, SCN2B, SCN3B and a combination thereof.

23. The method according to claim 20, wherein the gene encoding potassium (K⁺)-adenosine triphosphate channels are ABCC9.

24. The method according to claim 20, wherein the SNP on chromosome 4q25 is rs2200733-T allele.

25. The method according to claim 1, wherein the atrial fibrillation occurs postoperatively.

26. The method according to claim 1, further comprising administering an additional therapeutic agent systemically.

27. The method according to claim 26, wherein the additional therapeutic agent is selected from the group consisting of a statin, an anti-inflammatory agent, a thiazolidinedione, an analgesic agent, an anti-infective agent and a combination thereof.

28. A malleable drug delivery system for reducing incidence or severity of atrial fibrillation in a subject at risk thereof, comprising
(a) a particulate formulation containing a plurality of particles comprising a therapeutic amount of an anti-arrhythmic agent and a pharmaceutically acceptable carrier;
the malleable drug delivery system characterized by:
(i) its ability to contact a surface of a tissue susceptible to atrial fibrillation, adhere to the surface susceptible to atrial fibrillation; conform to contours of the surface susceptible to atrial fibrillation; or a combination thereof; and
(ii) release of the therapeutic agent at the implant site may be effective to produce a predominantly localized pharmacologic effect over a desired amount of time, where the desired amount of time is the time necessary to reduce the incidence or severity of atrial fibrillation.

29. The malleable drug delivery system according to claim 28, wherein the anti-arrhythmic agent is selected from the group consisting of a Class I anti-arrhythmic agent, a Class II antiarrhythmic agent, a Class III anti-arrhythmic agent, a Class IV anti-arrhythmic agent, a Class V anti-arrhythmic agent and a combination thereof.

30. The malleable drug delivery system according to claim 29, wherein the anti-arrhythmic agent is a Class III anti-arrhythmic agent.

31. The malleable drug delivery system according to claim 30, wherein the Class III anti-arrhythmic agent is amiodarone, a derivative of amiodarone, a metabolite of amiodarone, an analog of amiodarone or a combination thereof.

32. The malleable drug delivery system according to claim 31, wherein the derivative of amiodarone is dronedarone [n-(2-butyl-3-(4-(3-dibutylaminopropoxy)-benzoyl)benzofuran-5-yl)-methanesulfonamide] or KB130015 (KB015) [2-methyl-3-(3,5-diiodo-4-carboxymethoxybenzyl)benzofuran].

33. The malleable drug delivery system according to claim 31, wherein the metabolite of amiodarone is mono-N-desethylamiodarone (B2-O-Et-NH-ethyl), di-N-desethylamiodarone (B2-O-Et-NH₂) or (2-butyl-benzofuran-3-yl)-(4-hydroxy-3,5-diiodophenyl)-methanone (B2) carrying an ethanol side chain [(2-butylbenzofuran-3-yl)-[4-(2-hydroxyethoxy)-3,5-diiodophenyl]-methanone (B2-O-Et-OH)].

34. The malleable drug delivery system according to claim 31, wherein the analog of amiodarone is N-dimethylamiodarone (B2-O-Et-N-dimethyl), N-dipropylamiodarone (B2-O-Et-N-dipropyl), B2-O-carrying an acetate side chain [[4-(2-butyl-benzofuran-3-carbonyl)-2,6-diiodophenyl]-acetic acid; B2-O-acetate], B2-O-Et carrying an propionamide side chain (B2-O-Et-propionamide), or B2-O carrying an ethyl side chain [(2-butylbenzofuran-3-yl)-(4-ethoxy-3,5-diiodophenyl)-methanone; B2-O-Et].

35. The malleable drug delivery system according to claim 28, wherein the surface susceptible to atrial fibrillation is an atrium a superior pulmonary vein, a superior vena cava vein or a coronary sinus.

36. The malleable drug delivery system according to claim 28, further comprising releasing one-half of the therapeutic agent from the delivery system at the implant site within 6 hours; within 12 hours to 4 days; or within 3 to 5 days in vivo.

37. The malleable drug delivery system according to claim 28, wherein the particulate formulation is in form of a filament, a cord, a thread, a string, a film, a sheet or a patch.

38. The malleable drug delivery system according to claim 28, wherein the therapeutic agent is dispersed throughout the particle, adsorbed onto the particle, contained in a core surrounded by a coating.

39. The malleable drug delivery system according to claim 28, wherein a surface of the particle is impregnated with the therapeutic agent.

40. The malleable drug delivery system according to claim 28, wherein the particles comprise a matrix.

41. The malleable drug delivery system according to claim 40, wherein the matrix is impregnated with the therapeutic agent.

42. The malleable drug delivery system according to claim 28, wherein the delivery system is impregnated with the therapeutic agent.

43. The malleable drug delivery system according to claim 28, wherein the therapeutic agent is entrapped by the malleable delivery system.

44. The malleable drug delivery system according to claim 28, wherein the therapeutic agent is released from the malleable delivery system into the implant site.

45. The malleable drug delivery system according to claim 28, wherein the atrial fibrillation is a consequence of cardiac surgery, etiological factors or genetic factors.

46. The malleable drug delivery system according to claim 45, wherein the etiological factors are selected from the group consisting of age, structural remodeling, congestive heart failure (CHF), hypertension, valvular heart disease, coronary artery disease (CAD), peri- or myocarditis, atrial myxomas, hypertrophic cardiomyopathy, alcohol consumption, hyperthyroidism, sleep apnea, obesity and a combination thereof.

47. The malleable drug delivery system according to claim 45, wherein the genetic factors are selected from the group consisting of a gene encoding myocardial potassium (K⁺) channels, a gene encoding sodium (Na⁺) channels, a gene encoding potassium (K⁺)-adenosine triphosphate channels, nucleoporin-155 (NUP155), gap junction protein connexion 40 (GJAS), atrial natriuretic peptide (NPPA), a single-nucleotide polymorphism (SNP) on chromosome 4q25 and a combination thereof.

48. The malleable drug delivery system according to claim 47, wherein the gene encoding myocardial potassium (K⁺) channels are selected from the group consisting of KCNQ1, KCNA5, KCNE5, KCNJ2, KCNE2 and a combination thereof.

49. The malleable drug delivery system according to claim 47, wherein the gene encoding sodium (Na⁺) channels are selected from the group consisting of SCN5A, SCN1B, SCN2B, SCN3B and a combination thereof.

50. The malleable drug delivery system according to claim 47, wherein the gene encoding potassium (K⁺)-adenosine triphosphate channels are ABCC9.

51. The malleable drug delivery system according to claim 47, wherein the SNP on chromosome 4q25 is rs2200733-T allele.

52. The malleable drug delivery system according to claim 28, wherein the atrial fibrillation is postoperative.

53. The malleable drug delivery system according to claim 28, wherein the malleable delivery system further comprises an additional therapeutic agent.

54. The malleable drug delivery system according to claim 53, wherein the additional therapeutic agent is selected from the group consisting of a statin, an anti-inflammatory agent, a thiazolidinedione, an analgesic agent, an anti-infective agent and a combination thereof.
